(19) United States
(12) Patent Application Publication
(10) Pub. No.: US 2025/0223594 A1
(43) Pub. Date: Jul. 10, 2025

(54) MODIFIED DOUBLE-STRANDED OLIGONUCLEOTIDE MOLECULE, MODIFIED DOUBLE-STRANDED OLIGONUCLEOTIDE CONJUGATE, AND USE THEREOF

(71) Applicant: RIGERNA THERAPEUTICS (BEIJING) CO., LTD., Beijing (CN)

(72) Inventors: Yuanyu Huang, Beijing (CN); Yongxin Gao, Beijing (CN); Xiaofeng Han, Beijing (CN)

(21) Appl. No.: 19/006,299

(22) Filed: Dec. 31, 2024

Related U.S. Application Data
(63) Continuation-in-part of application No. PCT/CN2023/137141, filed on Dec. 7, 2023.

(30) Foreign Application Priority Data
Dec. 28, 2022 (CN) 202211694998.0
Nov. 8, 2023 (CN) 202311478409.X

(51) Int. Cl. C12N 15/113

(52) U.S. Cl. CPC C12N 15/113; C12N 2310/14; C12N 2310/321; C12N 2310/351

(57) ABSTRACT

Provided are modified double-stranded oligonucleotide (dsRNA) molecule, double-stranded oligonucleotide conjugate, and use thereof, which are related to technical field of nucleic acid drugs. Double-stranded oligonucleotide comprises sense strand and an antisense strand; positions 1-9 counting from 5′ end of antisense strand comprise at least one modified nucleotide N_{M}, nucleotide N_{M} being nucleotide having modified 2′-silicon-atom-containing group; and spatial volume of 2′-silicon-atom-containing group being greater than that of 2′-methoxy group (2′-OMe). Double-stranded oligonucleotide modified using said method can maintain activity and Tm value of double-stranded oligonucleotide molecules while effectively reducing off-target effects thereof. Double-stranded oligonucleotide conjugate comprises conjugate group and double-stranded oligonucleotide molecule, double-stranded oligonucleotide molecule comprises sense strand and an antisense strand, and at least one of positions 1-9 counting from 5′-end of antisense strand comprises nucleotide modified by 2′-(O)ₘ(CH₂)ₙOR₁ group.

### CROSS-REFERENCE TO RELATED APPLICATIONS

The present application is a continuation-in-part application of PCT application No. PCT/CN2023/137141 filed on Dec. 7, 2023, which claims the priority to the Chinese patent application with the filing No. 202211694998.0, filed on Dec. 28, 2022 with the Chinese Patent Office and entitled “MODIFIED DOUBLE-STRANDED OLIGONUCLEOTIDE MOLECULE AND USE THEREOF”, and the Chinese patent application with the filing No. 202311478409.X, filed on Nov. 8, 2023 with the Chinese Patent Office and entitled “MODIFIED DOUBLE-STRANDED OLIGONUCLEOTIDE CONJUGATE COMPOSITION AND USE THEREOF”, the contents of which are incorporated herein by reference in entirety.

### REFERENCE TO SEQUENCE LISTING

This application includes a Sequence Listing filed electronically as an XML file named “Substitute sequence listing_CHOFN-24233-USCIP.xml”, created on Apr. 3, 2025, with a size of 488,242 bytes. The Sequence Listing is incorporated herein by reference.

### TECHNICAL FIELD

The present disclosure relates to the technical field of nucleic acid drugs, and particularly to a double-stranded oligonucleotide molecule having a specific modification motif and capable of reducing an off-target effect, a modified double-stranded oligonucleotide conjugate, a composition containing the conjugate and use thereof.

### BACKGROUND ART

Off-target toxicity is one of the key problems and challenges in the research and development of small nucleic acid drugs. Those skilled in the art have been endeavoring to address drug-formation obstacles of small nucleic acid drugs caused by an off-target effect, but many nucleic acid drugs can hardly be used in practical research and development of drugs due to toxicity generated by the off-target effect. The off-target toxicity of the small nucleic acid drugs is mainly derived from toxicity reaction triggered by drug molecules binding to non-target RNA, while a main off-target effect of siRNA is derived from pairing of an antisense seed region (sites 1-9 from 5′-end) with non-target mRNA, and gene expression abnormality (miRNA-like off-target effect) caused by an action mechanism similar to microRNA (miRNA). Many nucleic acid drugs having excellent activity in preclinical drug efficacy study are difficult to use in practical drug research and development due to toxicity caused by the off-target effect thereof.

Chemical modification techniques of the small nucleic acid drugs can improve stability and efficacy of such type of drugs, avoid immune responses, and at the same time are also main means to reduce the off-target effect. In research in the art, technical features of existing solutions for attenuating off-target effect of siRNA are all realized by means of attenuating thermodynamic stability (Tm value is reduced after chemical modification) of seed region having a potential negative impact on physicochemical properties such as activity, that is, by means of thermally unstable nucleotide modification of the antisense seed region, binding of the seed region of the siRNA antisense strand to an off-target gene is attenuated, thereby relieving occurrence of miRNA-like off-target effect. For example, without substantially changing contents and sites of double-stranded phosphorothioate (PS), methoxy (Omc) and fluoro (F) modifications, a single GNA modification (siRNA modification strategy is as shown in FIG. 9, and GNA structure is as shown in FIG. 10) is introduced to nucleotide at site 7 from 5′-end of a seed region of siRNA antisense strand, which, depending on different substitution bases, can significantly reduce Tm value of double strands to 4.3-16.8° C., thus selectively relieving the miRNA-like off-target effect mediated by the seed region.

In addition, there is also an anti-off-target strategy of using UNA modification on nucleotide at site 7 from the 5′-end of the seed region of the antisense strand (see FIG. 10 for UNA structure). Alternatively, a strategy of performing F substitution and methyl disubstitution modification at 2′-site of sugar ring of nucleotide at this site (see FIG. 10 for structures of F substitution and methyl disubstitution) likewise also significantly reduces the Tm value of the double strands to 15° C., so as to attenuate the thermodynamic stability of the seed region and achieve the purpose of relieving the off-target effect.

Chemical modification techniques can improve stability and efficacy of such type of drugs, avoid immune responses, and at the same time are also main means to reduce the off-target effect. For example, introduction of a specific modified nucleotide monomer to the seed region of the siRNA antisense strand can relieve the off-target effect mediated by the seed region. In addition, high-efficiency delivery carriers are also important components of oligonucleotide drugs. Asialoglycoprotein receptor (ASGPR) ligand N-acetylgalactosamine (GalNAc) can promote delivery of oligonucleotide drugs to hepatocytes. Multivalent optimized GalNAc ligands have higher binding affinity to ASGPR, and are delivered to hepatocytes more efficiently.

It is an important problem to be solved urgently in the art to reduce the off-target effect of siRNA drugs as much as possible while maintaining good pharmaceutical activity and thermodynamic stability (Tm value) of this type of drugs; or to reduce the off-target effect of siRNA drugs as much as possible while maintaining good pharmaceutical activity of this type of drugs and high-efficiency delivery carriers.

### SUMMARY

According to one aspect of the present disclosure, the present disclosure provides a modified double-stranded oligonucleotide (dsRNA) molecule, which contains a sense strand and an antisense strand, wherein each nucleotide of the sense strand and the antisense strand is independently a modified or unmodified nucleotide. Nucleotide sequences of the sense strand and the antisense strand are at least partially reversely complementary. Moreover, the nucleotide sequence of the antisense strand is at least partially reversely complementary to a target gene mRNA.

Specifically, in the dsRNA molecule of the present disclosure, positions of sites 1-9 (preferably sites 2-9) of the antisense strand, counting from a 5′-end, contain at least one modified nucleotide N_{M}, wherein the nucleotide N_{M} is a nucleotide modified with 2′-bulky group, and the 2′-bulky group contains an Si atom and is greater than 2′-methoxy group (2′-OMe) in steric bulk. In some examples, the modified nucleotide N_{M} is located at positions of sites 3-9 of the dsRNA antisense strand from the 5′-end. In some other examples, the modified nucleotide N_{M} is located at positions of sites 5-8 of the antisense strand from the 5′-end. In some other examples, the modified nucleotide N_{M} is located at positions of sites 5-7 of the antisense strand from the 5′-end. In the above, the modified nucleotide N_{M} will not cause the dsRNA where it is located to have an obviously or significantly reduced total melting temperature (Tm).

In some embodiments of the present disclosure, the modified nucleotide N_{M} is a 2′-O—Si-modified nucleotide.

In some other embodiments of the present disclosure, the modified nucleotide N_{M} has a structure as represented by following Formula (I), or is a tautomer of Formula (I):

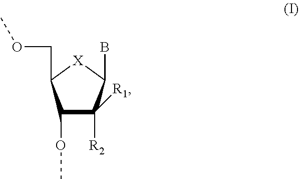

where X is selected from the group consisting of CH₂, O, S or NH;
B is selected from the group consisting of nucleotide bases, heteroaryls having 6-12 carbon atoms, heterocyclic bases or base substitution groups;
the nucleotide bases include, but are not limited to, uracil or derivatives thereof, thymine or derivatives thereof, cytosine or derivatives thereof, 5-methylcytosine or derivatives thereof, adenine or derivatives thereof, or guanine or derivatives thereof; and
R₁ and R₂ are each independently H or —(O)ₘ(CH₂)ₙOR₃, and R₁ and R₂ are not both H or —(O)ₘ(CH₂)ₙOR₃; m is selected from 0 or 1, and n is an integer selected from 0 to 3, wherein a steric bulk (or steric hindrance) of —(O)ₘ(CH₂)ₙOR₃ substituent is greater than a steric bulk (or steric hindrance) of methoxy. Further, R₃ is a Si-containing group.

In the above, dashed lines (---) in Formula (I) represent linkage positions for linking with an adjacent nucleotide.

In some embodiments, B is a nucleotide base or derivatives thereof. Further, B is selected from the group consisting of uracil, thymine, cytosine, 5-methylcytosine, adenine or guanine.

In some embodiments, n is an integer selected from 0 to 2.

In some embodiments, n is selected from 0 or 1.

In some embodiments, R₃ is —Si(R₄)₃, where each R₄ is independently selected from the group consisting of: substituted or unsubstituted C1-C6 alkyl, or substituted or unsubstituted C1-C6 alkoxy; and if R₄ contains a substituent, this substituent is selected from the group consisting of C1-C6 alkyl, hydroxy, halogen, alkoxy having no more than 6 carbon atoms, amino, cycloalkyl having no more than 12 carbon atoms, aryl having no more than 12 carbon atoms or heteroaryl having no more than 12 carbon atoms. Further, R₃ is a TBDMS or TIPS group. In some embodiments, R₁ or R₂ is a —O-TOM group.

In one or more embodiments, the nucleotide N_{M} has a structure as represented by following Formula (II), or is a tautomer of Formula (II):

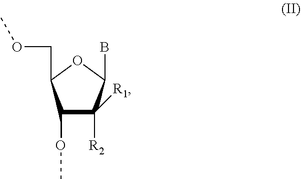

where B is selected from the group consisting of: uracil or derivatives thereof, thymine or derivatives thereof, cytosine or derivatives thereof, 5-methylcytosine or derivatives thereof, adenine or derivatives thereof, or guanine or derivatives thereof; and
R₁ and R₂ are each independently H or —(O)ₘ(CH₂)ₙOR₃, and R₁ and R₂ are not both H or —(O)ₘ(CH₂)ₙOR₃; m is selected from 0 or 1, and n is an integer selected from 0 to 2, wherein a steric bulk of —(O)ₘ(CH₂)ₙOR₃ substituent is greater than that of methoxy.

In the above, R₃ is —Si(R₄)₃, where each R₄ is independently selected from the group consisting of: substituted or unsubstituted C1-C6 alkyl, or substituted or unsubstituted C1-C6 alkoxy, wherein the substituent is selected from the group consisting of C1-C6 alkyl, hydroxy, halogen, alkoxy having no more than 6 carbon atoms, amino, cycloalkyl having no more than 12 carbon atoms, aryl having no more than 12 carbon atoms or heteroaryl having no more than 12 carbon atoms.

In some embodiments, n is 0 or 1.

In some embodiments, R₁ or R₂ is a —O-TOM group, and R₁ and R₂ are not both —O-TOM group.

In some embodiments, the nucleotide N_{M} has a structure as represented by following Formula (III), or is a tautomer of Formula (III):

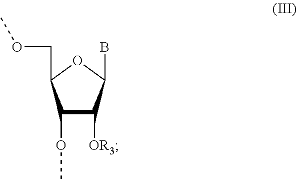

B is selected from the group consisting of uracil, thymine, cytosine, 5-methylcytosine, adenine or guanine; and
R₃ is —Si(R₄)₃, where each R₄ is independently selected from the group consisting of: substituted or unsubstituted C1-C6 alkyl, or substituted or unsubstituted C1-C6 alkoxy, wherein a substituent is selected from the group consisting of C1-C6 alkyl, hydroxy, halogen, alkoxy having no more than 6 carbon atoms or amino.

In some embodiments, R₃ or is selected from TBDMS group or TIPS group.

In some embodiments, in addition to the nucleotide N_{M}, the double-stranded oligonucleotide further contains other modified nucleotides, wherein the other modified nucleotides include the group consisting of: abasic nucleotides, reverse abasic nucleotides, reverse deoxyribonucleotides, 2′-halogen modified nucleotides, 2′-methoxy modified nucleotides, 2′-deoxy (i.e., 2′-H) modified nucleotides, 2′-O-methoxyalkyl modified nucleotides, 2′-O-alkyl modified nucleotides, 2′-O-allyl modified nucleotides, 5′-vinyl modified nucleotides, 5′-cyclopropyl modified nucleotides, BNA, LNA, 5′-vinylphosphate modified nucleotides, 5′-cyclopropylphosphate modified nucleotides or 5′-alkyl substituent phosphate modified nucleotides. In some embodiments, the other modified nucleotides are one or more selected from the group consisting of 2′-methoxy modified nucleotides, 2-halogen modified nucleotides and 2′-deoxy modified nucleotides.

In some embodiments, the sense strand and antisense strand of the dsRNA both contain a 2′-methoxy modified nucleotide and a 2′-halogen modified nucleotide. In some embodiments, the sense strand and antisense strand of the dsRNA each independently contain no more than four 2′-fluoro modified nucleotides; and no less than five 2′-methoxy modified nucleotides. In some embodiments, the sense strand and antisense strand of the dsRNA each independently contain no more than five 2′-fluoro modified nucleotides. In one or more embodiments, the number of 2′-fluoro modification in the sense strand or antisense strand is each independently no more than five, no more than four, no more than three, or no more than two. In one or more embodiments, the number of 2′-fluoro modification in the sense strand or the antisense strand is each independently five, four, three, two or one.

In some embodiments, the sense strand or antisense strand of the dsRNA contains 14-40 nucleotides. Further, the sense strand or antisense strand of the dsRNA contains 14-30 nucleotides. Still further, the sense strand or antisense strand of the dsRNA contains 17-25 nucleotides. In some embodiments, the sense strand contains 17-23 nucleotides, and the antisense strand contains 19-23 nucleotides.

In some embodiments, the double-stranded oligonucleotide contains a duplex region of 19-21 base pairs.

In some embodiments, at least one linkage in the double-stranded oligonucleotide is modified; and preferably, modification of the linkage includes at least one of phosphorothioate internucleotide linkage modification or methylphosphonate internucleotide linkage modification.

In some embodiments, the double-stranded oligonucleotide further may be linked with a ligand. In some embodiments, the ligand is selected from the group consisting of galactose, galactosamine, N-acetylgalactosamine, or derivatives thereof.

In the above, the ligand is covalently attached to the 5′-end or 3′-end of the sense strand of the double-stranded oligonucleotide by a linker.

In some embodiments, the double-stranded oligonucleotide molecule is represented by following Formula (A), and the double-stranded oligonucleotide contains an antisense strand represented by Formula A2 and a sense strand represented by Formula A1;

5′-(N_{A})ₓ—(N_{L})ₛ—(N_{L2})_{y}-3′; Formula A1:

3′-(N_{B})_{z}—(N_{L})ₜ—[N_{L}(N_{M})]₈—N_{L1}-5′(A); Formula A2:
where x is an integer selected from 0 to 6; s is an integer selected from 10 to 40; y is an integer selected from 0 to 3; z is an integer selected from 0 to 6; and t is an integer selected from 10 to 30.
(N_{L2})_{y} is an overhang nucleotide that does not undergo base complementary pairing with a nucleotide in the antisense strand.

Each nucleotide N_{L} is independently selected from unmodified nucleotides or from following modified nucleotides: abasic nucleotides, reverse abasic nucleotides, reverse deoxyribonucleotides, 2′-halogen modified nucleotides, 2′-O-methoxyalkyl modified nucleotides, 2′-O-alkyl modified nucleotides, 2′-O-allyl modified nucleotides, 5′-vinyl modified nucleotides, 5′-cyclopropyl modified nucleotides, BNA, LNA or 2′-deoxy modified nucleotides.

[N_{L}(N_{M})]₈ is five contiguous nucleotides composed of the nucleotide N_{L} and the nucleotide N_{M} located at sites 2-9 of the antisense strand from the 5′-end, and contains at least one nucleotide N_{M} therein. The nucleotide N_{M} is located at any position of sites 2-9 of the antisense strand counting from the 5′-end; and each nucleotide N_{M} is independently selected from Formula (I), Formula (II) or Formula (III) above.

Each nucleotide N_{A} is independently selected from unmodified nucleotides or from following modified nucleotides: abasic nucleotides, reverse abasic nucleotides, reverse deoxyribonucleotides, 2′-halogen modified nucleotides, 2′-O-methoxyalkyl modified nucleotides, 2′-O-methyl modified nucleotides, 2′-O-allyl modified nucleotides or 2′-deoxy modified nucleotides.

Each nucleotide N_{B} is independently an unmodified nucleotide or selected from following modified nucleotides: abasic nucleotides, reverse abasic nucleotides, reverse deoxyribonucleotides, 2′-halogen modified nucleotides, 2′-O-methoxyalkyl modified nucleotides, 2′-O-methyl modified nucleotides, 2′-O-allyl modified nucleotides or 2′-deoxy modified nucleotides.

The nucleotide NL₁ is a nucleotide defined by the nucleotide N_{L}, or is selected from following nucleotides: 5′-vinylphosphate modified nucleotides, 5′-cyclopropylphosphate modified nucleotides or 5′-alkyl substituent phosphate modified nucleotides.

Each nucleotide N_{L2} is independently an unmodified nucleotide or selected from following modified nucleotides: abasic nucleotides, reverse abasic nucleotides, reverse deoxyribonucleotides, 2′-halogen modified nucleotides, 2′-O-methoxyalkyl modified nucleotides, 2′-O-alkyl modified nucleotides, 2′-O-allyl modified nucleotides, 2′-vinyl modified nucleotides, 2′-cyclopropyl modified nucleotides, BNA, LNA or 2′-deoxy modified nucleotides.

In some embodiments, in the [N_{L}(N_{M})]₈, the nucleotide N_{M} is located at any position of sites 3-8 of the antisense strand counting from the 5′-end.

In some embodiments, when the nucleotide N_{L} is a modified nucleotide, each nucleotide N_{L1} is independently selected from following modified nucleotides: 2′-methoxy modified nucleotides, 2′-fluoro modified nucleotides or 2′-deoxy (i.e., 2′-H) modified nucleotides.

In some embodiments, each nucleotide N_{M} is independently selected from the nucleotides represented by Formula (I), Formula (II) or Formula (III) above.

According to another aspect of the present disclosure, the present disclosure further provides a pharmaceutical composition containing a therapeutically effective amount of the above double-stranded oligonucleotide molecule and a pharmaceutically acceptable adjuvant.

In some embodiments, the pharmaceutical composition is used for preventing, relieving or treating pathological conditions or diseases caused by expression of a specific gene.

In some embodiments, the pharmaceutical composition delivers the above dsRNA molecule to a tissue or cell of a subject, particularly liver site.

According to another aspect of the present disclosure, the present disclosure further provides use of the above dsRNA molecule or the above pharmaceutical composition in the preparation of drugs for treating and/or preventing pathological conditions or diseases caused by expression of a specific gene.

According to another aspect of the present disclosure, the present disclosure further provides a method for inhibiting target gene expression, wherein the method includes administering to a subject the above double-stranded oligonucleotide molecule, or the above pharmaceutical composition.

The present disclosure further provides a double-stranded oligonucleotide conjugate having a reduced off-target effect and a modification method thereof, as well as a double-stranded oligonucleotide conjugate for intrahepatic delivery and a composition thereof.

According to another aspect of the present disclosure, the present disclosure provides a double-stranded oligonucleotide conjugate containing a conjugate group and a double-stranded oligonucleotide molecule.

In some embodiments, the double-stranded oligonucleotide molecule contains a duplex formed by at least partial complementation of a sense strand and an antisense strand, wherein the sense strand and the antisense strand each have 17-35 nucleotides; sites 1-9 of the antisense strand counting from a 5′-end contain at least one nucleotide modified with 2′-(O)ₘ(CH₂)ₙOR₁ group, where m is selected from 0 or 1, n is an integer selected from 0 to 3, and R₁ is a Si-containing group.

In some embodiments, n is selected from 0 or 1.

In some embodiments, the conjugate group is an asialoglycoprotein receptor (ASGPR) ligand group containing a galactosamine derivative unit. In some embodiments, the galactosamine derivative is selected from the group consisting of galactosamine, N-formylgalactosamine, N-acetylgalactosamine, N-propionylgalactosamine, N-n-butyrylgalactosamine or N-isobutyrylgalactosamine.

In some embodiments of the present disclosure, the double-stranded oligonucleotide conjugate has a structure represented by following Formula (IV):

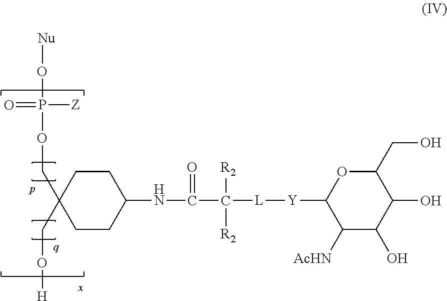

In Formula (IV), Nu represents the double-stranded oligonucleotide molecule;
x is selected from 1, 2, 3 or 4;
each Z is independently selected from hydroxyl or thiol;
each p is independently selected from 1, 2 or 3;
each q is independently selected from 1, 2 or 3;
each R₂ is independently selected from the group consisting of H, an optionally substituted C₁-C₆ alkyl, a C₁-C₆ haloalkyl or an optionally substituted C₁-C₆ alkoxy;
L is selected from the group consisting of C₁-C₂₀ alkylene, —(O—CH₂—CH₂—)ⱼ—, C₁-C₂₀ alkylene substituted with one or more substituents of O, S, NH or —NH—C(O)—, or

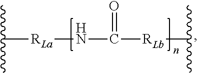

where R_{La} and R_{Lb} are each independently selected from optionally substituted C₁₋₁₀ alkylene, k is selected from 1, 2 or 3, and j is an integer selected from 0 to 10; and
each Y is independently selected from —NH—, O or S.

In some embodiments, L is selected from C₂-C₁₀ alkylene.

In some embodiments, L selected from

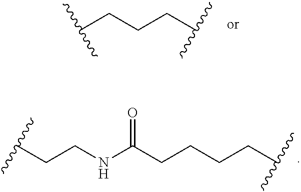 [SMILES: *CCC* ; *CCCCC(=O)NCC*]

In some embodiments, Y is selected from O.

In some embodiments, Z is selected from hydroxyl.

In some embodiments, R₂ is H.

In some embodiments, the conjugate group has a structure represented by following Formula (V):

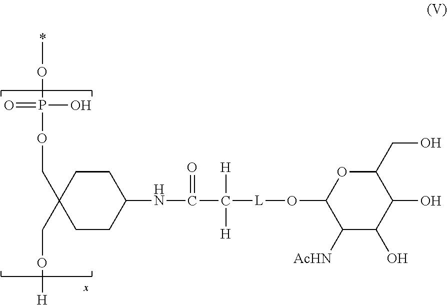

where * represents a covalent linking site of the conjugate group and the double-stranded oligonucleotide molecule;
x is an integer selected from 1 to 3; and
L is an alkyl chain having 2 to 10 carbon atoms.

In some embodiments, L is C₂-C₁₀ linear alkyl.

In some embodiments, the conjugate group is any one selected from following structures, or a stereoisomer thereof, or a pharmaceutically acceptable salt thereof:

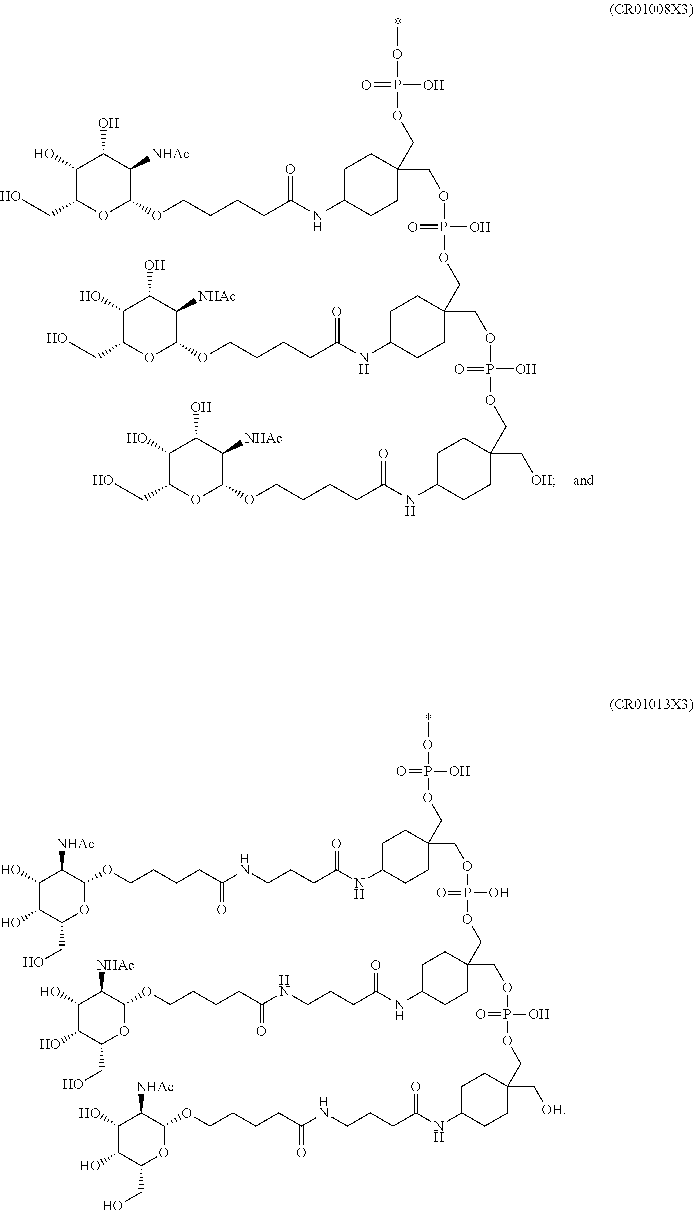

In some embodiments, R₁ is —Si(R₃)₃, where each R₃ is independently selected from the group consisting of substituted or unsubstituted C1-C6 alkyl, or substituted or unsubstituted C1-C6 alkoxy; and if R₃ contains a substituent, this substituent is selected from the group consisting of C1-C6 alkyl, hydroxy, halogen, amino or alkoxy having no more than 6 carbon atoms.

In some embodiments, R₁ is selected from TBDMS group or TIPS group.

In some embodiments, m and n are both 0.

In some embodiments, m and n are both 1.

In some embodiments, the 2′-(O)ₘ(CH₂)ₙOR₁ group is selected from 2′-O-TOM group, 2′-O-TBDMS group or 2′-O-TIPS group.

In the above, a structural formula of TBDMS is as represented by following Formula (a), a structural formula of the TIPS group is as represented by following Formula (B), and a structural formula of the TOM group is as represented by following Formula (Y):

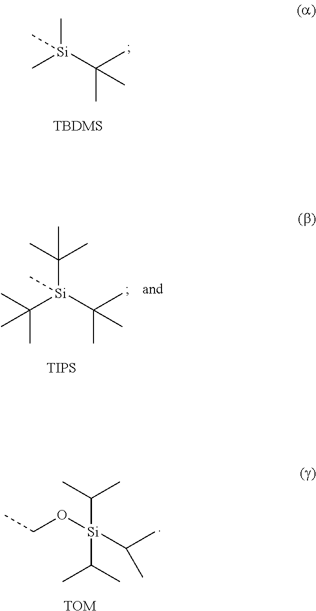

In some embodiments, each nucleotide in the duplex of the double-stranded oligonucleotide is modified.

In some embodiments, in a direction from the 5′-end to the 3′-end, at least three nucleotides at sites 7-10 of the sense strand are 2′-fluoro modified nucleotides, and nucleotides at remaining positions are 2′-O-methyl modified nucleotides; and/or in the direction from the 5′-end to the 3′-end, at least one nucleotide at sites 2-9 of the antisense strand is selected from nucleotides modified with the 2′-(O)ₘ(CH₂)ₙOR group.

In some embodiments, at least four nucleotides at sites 2, 6, 9, 12, 14 and 16 of the antisense strand are 2′-fluoro modified nucleotides, nucleotide at site 15 is selected from 2′-O-methoxyethyl modified nucleotide or 2′-O-methyl modified nucleotide, and nucleotides at remaining positions are 2′-O-methyl modified nucleotides.

In some embodiments, in the direction from the 5′-end to the 3′-end, at least three nucleotides at sites 7-10 of the sense strand are 2′-fluoro modified nucleotides, and nucleotides at remaining positions are independently selected from 2′-O-methyl modified nucleotides; and/or in the direction from the 5′-end to the 3′-end, at least one nucleotide at sites 3-8 of the antisense strand is selected from 2′-(O)ₘ(CH₂)ₙOR₁ group modified nucleotide, at least four of nucleotides at sites 2, 6, 9, 12, 14 and 16 are selected from 2′-fluoro modified nucleotides, nucleotide at site 15 is selected from 2′-O-methoxyethyl modified nucleotide or 2′-O-methyl modified nucleotide; and nucleotides at remaining positions are 2′-O-methyl modified nucleotides.

In some embodiments, in the direction from the 5′-end to the 3′-end, nucleotides at sites 7-10 of the sense strand are 2′-fluoro modified nucleotides, and nucleotides at remaining positions are independently selected from 2′-O-methyl modified nucleotides; and in the direction from the 5′-end to the 3′-end, one nucleotide at sites 3-8 of nucleotide sequence of the antisense strand is selected from 2′-(O)ₘ(CH₂)ₙOR, group modified nucleotide, at least five of nucleotides at sites 2, 6, 12, 14 and 16 are selected from 2′-fluoro modified nucleotides, nucleotide at site 15 is selected from 2′-O-methoxyethyl modified nucleotide, and nucleotides at remaining positions are independently selected from 2′-O-methyl modified nucleotides.

In some embodiments, the double-stranded oligonucleotide conjugate further contains one or more nucleotides containing 5′ phosphorothioate group.

In some embodiments, the conjugate group in the double-stranded oligonucleotide conjugate is covalently linked on the sense strand and/or antisense strand of the double-stranded oligonucleotide. In some embodiments, the conjugate group is covalently linked to an end of the sense strand.

According to another aspect of the present disclosure, the present disclosure provides a composition including the double-stranded oligonucleotide conjugate of the present disclosure.

In some embodiments, the composition further includes a pharmaceutically acceptable carrier.

According to another aspect of the present disclosure, the present disclosure provides use of the double-stranded oligonucleotide conjugate and/or the composition in the preparation of drugs for treating and/or preventing diseases or conditions associated with dysregulated mRNA level of specific gene expression.

According to another aspect of the present disclosure, the present disclosure provides a method for regulating specific gene expression in a target cell, wherein the method includes:
making the double-stranded oligonucleotide conjugate and/or the composition of the present disclosure contact the target cell.

According to another aspect of the present disclosure, the present disclosure provides a method for preventing and/or treating diseases or conditions associated with dysregulated mRNA level of specific gene expression in a target cell in a subject, wherein the method includes:
administering to the subject a pharmaceutically acceptable dose of the double-stranded oligonucleotide conjugate and/or the composition of the present disclosure.

According to another aspect of the present disclosure, the present disclosure provides a kit containing the double-stranded oligonucleotide conjugate and/or the composition of the present disclosure.

### BRIEF DESCRIPTION OF DRAWINGS

In order to more clearly illustrate technical solutions in the embodiments of the present disclosure or in the prior art, drawings which need to be used for describing the embodiments or the prior art will be briefly introduced below. Apparently, the drawings in the description below merely show some embodiments of the present disclosure, and those ordinarily skilled in the art still could obtain other drawings in light of these drawings without using any inventive efforts.

FIG. 1 shows relative expression levels of target gene CC3 mRNA after HepG2 is transfected with various test substances tested in Example 2;

FIG. 2 shows relative expression levels of target gene ANGPTL3 mRNA after free uptake of various test substances tested in Example 3 by mouse primary hepatocytes;

FIG. 3 shows relative expression levels of target gene FXI mRNA after free uptake of various test substances tested in Example 4 by mouse primary hepatocytes;

FIG. 4 shows relative expression levels of target genes of interest in mice after administration of various test substances tested in Example 5;

FIG. 5 shows relative expression levels of target genes of interest in mice after administration of various test substances tested in Example 6;

FIGS. 6A-6H show on-target activity IC50 curves detected by in-vitro psiCHECK system after administration of various test substances tested in Example 7, where FIGS. 6A-6H show test results of RX597001, RX597002, RX597003, RX597004, RX597005, RX597006, RX597007 and RX597008 in order;

FIGS. 7A-7H show off-target activity IC50 curves detected by in-vitro psiCHECK system after administration of various test substances tested in Example 8, where FIGS. 7A-7H show test results of RX597001, RX597002, RX597003, RX597004, RX597005, RX597006, RX597007 and RX597008 in order;

FIG. 8A shows detection results of ALT concentrations in ICR mouse serum after administration of various siRNA conjugates tested in Example 9;

FIG. 8B shows detection results of AST concentrations in ICR mouse serum after administration of various siRNA conjugates tested in Example 9;

FIG. 9 shows an anti-off-target strategy of GNA modification in a seed region of siRNA antisense strand;

FIG. 10 shows a GNA structural formula, a UNA structural formula, and a structural formula of F and methyl disubstitution;

FIG. 11 shows relative remaining expression levels of target gene ANGPTL3 mRNA after free uptake of various test substances tested in the present example by mouse primary hepatocytes; and

FIG. 12 shows relative remaining expression levels of target gene ANGPTL3 mRNA after free uptake of various test substances tested in the present example by mouse primary hepatocytes.

### DETAILED DESCRIPTION OF EMBODIMENTS

Technical solutions of the present disclosure will be described below clearly and completely in conjunction with examples. Apparently, only some but not all of the examples of the present disclosure are described. Based on the examples in the present disclosure, all of other examples obtained by those ordinarily skilled in the art without using any inventive efforts shall fall within the scope of protection of the present disclosure.

The present disclosure provides a modification method for a double-stranded oligonucleotide (dsRNA) molecule conducive to reducing an off-target effect, and a dsRNA molecule for use in reducing target gene expression and a composition thereof. Generally, the dsRNA molecule includes a sense strand and an antisense strand. By using a nucleotide modification strategy that substantially does not attenuate a Tm value on a specific position of dsRNA sequence, the present disclosure reduces the off-target effect of the dsRNA sequence and at the same time maintains good pharmacodynamic activity.

Thus, the first objective of the present disclosure is to provide a modified double-stranded oligonucleotide (dsRNA) molecule, so as to solve the technical problem of lacking a modification strategy that can reduce the off-target effect of dsRNA drugs while maintaining good pharmacodynamics activity and Tm value of this type of drugs existing in the prior art.

In the modified dsRNA of the present disclosure, a seed region of an antisense strand (i.e., at positions 1-9 of the antisense strand from a 5′-end) contains at least one nucleotide modified with 2′-bulky group, wherein the 2′-bulky group in the modified nucleotide contains a Si atom and has a steric bulk greater than that of 2′-OMe group. By performing the above group modification in the seed region of the antisense strand, binding and recognition of the dsRNA antisense strand to an off-target gene are attenuated by steric hindrance, thereby reducing the off-target effect.

The second objective of the present disclosure is to provide use of the above modified double-stranded oligonucleotide in inhibiting target gene expression or in preparing a pharmaceutical composition. The modified oligonucleotide is more effective in reducing an off-target effect than a parental dsRNA molecule lacking corresponding modification.

Compared with the prior art, the present disclosure has following beneficial effects.

In order to reduce the off-target effect of nucleic acid molecule of dsRNA drugs as much as possible while maintaining good pharmacodynamic activity of this type of drugs, the present disclosure adopts a novel modification strategy, that is, using a nucleotide N_{M} modified with a bulky group that does not attenuate the Tm value at a specific position of the antisense strand of dsRNA, can significantly reduce the off-target effect of the siRNA sequence, and at the same time maintain the good pharmacodynamic activity. Specifically, the modified double-stranded oligonucleotide provided by the present disclosure contains the sense strand and the antisense strand, wherein sites 1-9 of the antisense strand counting from the 5′-end contain at least one nucleotide N_{M}, the nucleotide N_{M} being a nucleotide modified with 2′-Si-containing group (wherein the 2′-Si-containing group has a greater steric hindrance than 2′-methoxy modification). The present disclosure found that the dsRNA containing at least one nucleotide N_{M} in a region of the sites 1-9 of the antisense strand counting from the 5′-end is more effective in reducing the off-target effect than the parental dsRNA molecule lacking corresponding modification.

When the double-stranded oligonucleotide inhibiting target gene expression provided by the present disclosure is applied to a method for preparing a pharmaceutical composition or inhibiting target gene expression, since the dsRNA can effectively inhibit target gene expression, and the anti-off-target effect thereof is more excellent, drugs containing the above dsRNA molecule can effectively reduce adverse effects of drugs caused by off-target.

According to some aspects of the present disclosure, while maintaining good pharmacodynamic activity and liver delivery efficiency, a double-stranded oligonucleotide conjugate of the present disclosure has a lower nucleic acid molecule off-target effect than a corresponding oligonucleotide molecule lacking corresponding modification, and can effectively reduce toxic and side effects of drugs caused by off-target.

In the present disclosure, the double-stranded oligonucleotide refers to a double-stranded structure formed by two oligonucleotides by complementary pairing of part or all of bases thereof, wherein the two oligonucleotides include a sense strand and an antisense strand, whose lengths may be the same or not. Oligonucleotides having a double-stranded structure belong to the double-stranded oligonucleotide of the present disclosure as long as there at least exists a region where bases are partially complementarily paired to form a duplex. The nucleotides forming the double-stranded oligonucleotide in the present disclosure may be modified or unmodified nucleotides, and when the nucleotides are modified nucleotides, unless otherwise noted, the modification in the present disclosure is not specified with a site of modification. For the double-stranded oligonucleotide in the present disclosure, in addition to the nucleotide being modified, a linkage linking nucleotides may also be modified, and double-stranded oligonucleotides containing a modified linkage between nucleotides also belong to the double-stranded oligonucleotide of the present disclosure. In addition to a nucleotide part, the double-stranded oligonucleotide in the present disclosure may also contain a compound molecule or a modifier acceptable in the art, so as to improve properties of the double-stranded oligonucleotide, for example, linked with a ligand to form a conjugate.

The nucleotide herein may refer to an independent nucleotide, or a nucleotide residue in an oligonucleotide; the nucleotide herein may be singular or plural, and when plural, it can refer to a class of nucleotides, for example, nucleotide N_{L} herein can refer to one nucleotide N_{L}, or several nucleotides N_{L} in the general formula of the dsRNA herein. The double-stranded oligonucleotide herein can also be represented by dsRNA, siRNA or double-stranded oligomeric nucleotide, and the double-stranded oligonucleotide, double-stranded oligomeric nucleotide, dsRNA and siRNA herein can be used interchangeably. 2-methoxy herein may also be represented by 2′-OMe, and 2-methoxy and 2′-OMe herein can be used interchangeably.

According to one aspect of the present disclosure, the present disclosure provides a modified double-stranded oligonucleotide molecule containing a sense strand and an antisense strand. In the above, the antisense strand has sufficient complementarity to a target sequence so as to mediate RNA interference. The double-stranded oligonucleotide contains, at sites 1-9 counting from a 5′-end of the antisense strand, at least one nucleotide N_{M} modified with 2′-bulky group, wherein the 2′-bulky group contains a Si atom and has a steric bulk greater than 2′-methoxy group (2′-OMe). The present disclosure found that containing at least one nucleotide N_{M} at sites 1-9 of the antisense strand counting from the 5′-end will not cause the dsRNA to have an obviously or significantly reduced total melting temperature (Tm), and is more effective in reducing an off-target effect than a parental dsRNA lacking corresponding modification. In the present disclosure, the double-stranded oligonucleotide contains at least one nucleotide N_{M} at sites 1-9 of the antisense strand counting from the 5′-end; and counting from the 5′-end of the antisense strand, at least one site of site 1, site 2, site 3, site 4, site 5, site 6, site 7, site 8 or site 9 of the antisense strand is nucleotide N_{M}. In some optional embodiments, at least one nucleotide N_{M} is located at sites 2-9. In some optional embodiments, at least one nucleotide N_{M} is located at sites 3-9. In some optional embodiments, at least one nucleotide N_{M} is located at sites 3-8. In some optional embodiments, at least one nucleotide N_{M} is located at sites 5-8. In some optional embodiments, at least one nucleotide N_{M} is located at sites 6-8.

In some optional embodiments, the modified nucleotide N_{M} is a 2′—O—Si— group modified nucleotide.

In some other optional embodiments, the nucleotide N_{M} is as represented by Formula (I), or is a tautomer as represented by Formula (I);

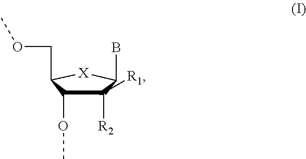

where X is CH₂, O, S or NH;
B is selected from the group consisting of nucleotide bases, heteroaryls having 6-12 carbon atoms, heterocyclic bases having 6-12 carbon atoms or base substitution groups having 6-12 carbon atoms; and
the nucleotide bases are selected from the group consisting of bases of uracil or derivatives thereof, thymine or derivatives thereof, cytosine or derivatives thereof, 5-methylcytosine or derivatives thereof, adenine or derivatives thereof, or guanine or derivatives thereof.

In some optional embodiments, B is selected from the nucleotide bases. In some specific embodiments, B is selected from the group consisting of uracil, thymine, cytosine, 5-methylcytosine, adenine or guanine.

In some optional embodiments, R₁ and R₂ are each independently H or —(O)ₘ(CH₂)ₙOR₃, and R₁ and R₂ are not both H or are not both-(O)ₘ(CH₂)ₙOR₃; m is selected from 0 or 1, and n is an integer selected from 0 to 3, R₃ is a Si-containing group and a steric bulk (or steric hindrance) of the —(O)ₘ(CH₂)ₙOR₃ substituent is greater than a steric bulk (or steric hindrance) of methoxy.

In some optional embodiments, n is an integer selected from 0 to 2.

In some optional embodiments, n is 0 or 1.

In some optional embodiments, R₃ is —Si(R₄)₃, where each R₄ is independently selected from the group consisting of: substituted or unsubstituted C1-C6 alkyl, or substituted or unsubstituted C1-C6 alkoxy; and if R₄ contains a substituent, this substituent is one or more selected from the group consisting of C1-C6 alkyl, hydroxy, halogen, alkoxy having no more than 6 carbon atoms, amino, cycloalkyl having no more than 12 carbon atoms, aryl having no more than 12 carbon atoms and heteroaryl having no more than 12 carbon atoms.

In some optional embodiments, R₃ is a TBDMS (tert-butyldimethylsilyl chloride) or TIPS (triisopropylsilyl) group. In the above, a structural formula of TBDMS is as represented by Formula (α), and a structural formula of the TIPS group is as represented by Formula (β):

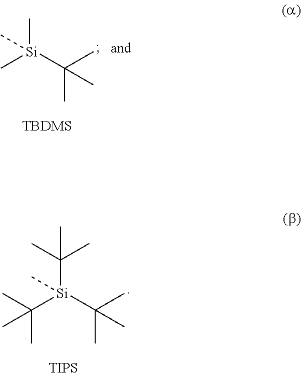

In some other optional embodiments, R₁ or R₂ is-O-TOM group, where TOM is triisopropylmethoxysilane, and R₁ and R₂ are not both —O-TOM group. In the above, a structural formula of the TOM group is as represented by Formula (γ):

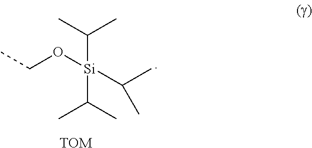

In one or more embodiments, X in Formula (I) is O.

In some optional embodiments, the nucleotide N_{M} is as represented by Formula (II), or is a tautomer as represented by Formula (II):

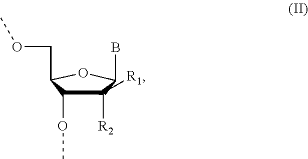

where B is a nucleotide base; and the nucleotide base is selected from the group consisting of uracil or derivatives thereof, thymine or derivatives thereof, cytosine or derivatives thereof, 5-methylcytosine or derivatives thereof, adenine or derivatives thereof, or guanine or derivatives thereof.

In some optional embodiments, R₁ and R₂ are independently selected from H or —(O)ₘ(CH₂)ₙOR₃, and R₁ and R₂ are not both H or —(O)ₘ(CH₂)ₙOR₃; m is selected from 0 or 1, and n is an integer selected from 0 to 2, wherein a steric bulk of —(O)ₘ(CH₂)ₙOR₃ substituent is greater than that of methoxy.

In some optional embodiments, n is 0 or 1.

In some optional embodiments, R₃ is —Si(R₄)₃, where each R₄ is independently selected from the group consisting of: substituted or unsubstituted C1-C6 alkyl, or substituted or unsubstituted C1-C6 alkoxy; and if R₄ contains a substituent, this substituent is selected from the group consisting of C1-C6 alkyl, hydroxy, halogen, alkoxy having no more than 6 carbon atoms, amino, cycloalkyl having no more than 12 carbon atoms, aryl having no more than 12 carbon atoms and heteroaryl having no more than 12 carbon atoms.

In some optional embodiments, R₁ or R₂ is selected from —O-TOM group, and R₁ and R₂ are not both —O-TOM group.

In one or more embodiments, one of R₁ and R₂ in Formula (II) is H.

In some optional embodiments, the nucleotide N_{M} is as represented by Formula (III), or is a tautomer as represented by Formula (III):

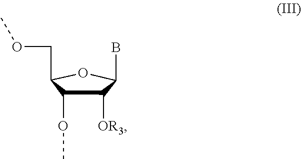

where B is a nucleotide base, and the nucleotide base is selected from the group consisting of uracil, thymine, cytosine, 5-methylcytosine, adenine or guanine; and
R₃ is —Si(R₄)₃, where each R₄ is independently selected from the group consisting of: substituted or unsubstituted C1-C6 alkyl, or substituted or unsubstituted C1-C6 alkoxy; and if R₄ contains a substituent, this substituent is selected from the group consisting of C1-C6 alkyl, hydroxy, halogen, alkoxy having no more than 6 carbon atoms or amino. In some optional embodiments, R₃ or is selected from TBDMS or TIPS.

In the above, dashed lines (---) in the above structural formulae (I), (II) and (III) represent linking positions. Moreover, substituents R₁ and R₂ at 2′-positions in the above structural formulae (I), (II) and (III) may have specific geometrical or stereoisomeric forms, including cis-trans isomers and other enantiomeric forms. In the double-stranded oligonucleotide provided by the present disclosure, except the nucleotide N_{M}, other nucleotides in the double-stranded oligonucleotide are optionally modified nucleotides or unmodified nucleotides; modifications of various nucleotides may be the same or different. Except the nucleotide N_{M}, the present disclosure does not limit distribution of other modified nucleotides and unmodified nucleotides in the double-stranded oligonucleotide. Optionally, the modified nucleotides may be in an alternating pattern. “Alternating” herein means that when one strand has two or more modifications, each or multiple modifications may occur at any nucleotide position on the strand at intervals, and the alternating pattern of modifications on the sense strand may be the same as or different from that on the antisense strand, and the alternating pattern of modifications on the sense strand can have an offset relative to the alternating pattern of modifications on the antisense strand.

In some optional embodiments, the sense strand and the antisense strand both may contain at least two nucleotide modifications. For example, 2′-O-methyl and 2′-fluoro modifications may occur on the sense strand or antisense strand in an alternating pattern. The alternating pattern of modifications on the sense strand may be the same as or different from that on the antisense strand, and the alternating pattern of modifications on the sense strand can have an offset relative to the alternating pattern of modifications on the antisense strand. Optionally, the modified nucleotides may be in a continuous mode, where continuous means that when a strand has two or more modifications, multiple modifications may be distributed adjacent to each other.

In the double-stranded oligonucleotide of the present disclosure, except the nucleotide N_{M}, other modifications in the double-stranded oligonucleotide can select a general and mature modification strategy for double-stranded oligonucleotides in the art, which is not limited in the present disclosure, and specific modifications include, but are not limited to, one or more of following modified nucleotides: abasic nucleotides, reverse abasic nucleotides, reverse deoxyribonucleotides, 2′-halogen modified nucleotides, for example, 2′-fluoro modified, 2′-methoxy modified nucleotides, 2′-OH modified nucleotides, 2′-H modified nucleotides, 2′-O-methoxyalkyl modified nucleotides, 2′-O-alkyl modified nucleotides, 2′-O-allyl modified nucleotides, 5′-vinyl modified nucleotides, 5′-cyclopropyl modified nucleotides, BNA, LNA, 5′-vinylphosphate modified nucleotides, 5′-cyclopropylphosphate modified nucleotides or 5′-alkyl substituent phosphate modified nucleotides; and further at least two selected from 2′-methoxy modified nucleotides, 2′-halogen modified nucleotides and 2′-deoxy modified nucleotides.

In one or more embodiments, the sense strand and the antisense strand each independently contain no more than five 2′-fluoro modified nucleotides. In an embodiment, the sense strand and the antisense strand each independently contain no less than five 2′-methoxy modifications.

In some optional embodiments, the sense strand and the antisense strand both contain 2′-methoxy modified nucleotide and 2-halogen modified nucleotide. Further, the sense strand and the antisense strand each independently contain no more than four 2′-fluoro modified nucleotides; and no less than five 2′-methoxy modifications. In some optional embodiments, linkages between various nucleotides in the double-stranded oligonucleotide can also be modified, the double-stranded oligonucleotide optionally at least contains one modified linkage, and modification of the linkage can be selected from phosphorothioate internucleotide linkage modification or methylphosphonate internucleotide linkage modification; and when the double-stranded oligonucleotide at least contains two modified linkages, modifications between various modified linkages may be the same or different. The modified linkage may occur at any nucleotide position of the sense strand or the antisense strand or both.

In some optional embodiments, in the double-stranded oligonucleotide, the antisense strand or the sense strand contains 14-40 nucleotides. In some optional embodiments, in the double-stranded oligonucleotide, the antisense strand or the sense strand contains 14-30 nucleotides. In some optional embodiments, in the double-stranded oligonucleotide, the antisense strand or the sense strand contains 17-25 nucleotides. In some optional embodiments, in the double-stranded oligonucleotide, the antisense strand or the sense strand contains 17-23 nucleotides.

In some optional embodiments, sequences of the antisense strand and the sense strand are sufficiently complementary to each other so as to form a duplex region of 19-21 base pairs.

In some other optional embodiments, the dsRNA molecule of the present disclosure contains a sense strand having a length of 17-23 nucleotides and an antisense strand having a length of 19-23 nucleotides, wherein at least one of modified nucleotides at positions 2-9 of the antisense strand counting from the 5′-end is selected from nucleotide N_{M} in a 2′—O—Si modified form; and other positions of the sense strand and the antisense strand also contain modified nucleotides selected from 2′-methoxy, 2′-fluoro, 2′-OH, 2′-H modified nucleotides, and the like.

In some optional embodiments, the double-stranded oligonucleotide further contains a ligand so as to form a dsRNA conjugate that promotes delivery or uptake of the dsRNA to or by a specific tissue or cell. The ligand includes, but is not limited to, galactose, galactosamine, N-acetylgalactosamine or derivatives thereof. The ligand is optionally covalently attached to the 5′-end or 3′-end of the sense strand of the double-stranded oligonucleotide by a linker.

In some optional embodiments, the 3′-end and/or 5′-end of the sense strand contains one or more overhang regions and/or capping groups. In some optional embodiments, the 3′-end and/or 5′-end of the antisense strand contains one or more overhang regions and/or capping groups. In the above, the overhang region optionally contains 1-6 nucleotides, and the overhang optionally mismatches with a target sequence, or is optionally complementarily paired with a base of the target sequence. In some optional embodiments, the modified double-stranded oligonucleotide contains a sense strand as represented by A1 and an antisense strand as represented by A2, and a general formula of this oligonucleotide is as represented by following Formula (A):

5′-(N_{A})ₓ—(N_{L})ₛ—(N_{L2})_{y}-3′; A1:

3′-(N_{B})_{z}—(N_{L})ₜ—[N_{L}(N_{M})]₈—N_{L1}-5′; A2: Formula (A),
where x and z are each an integer independently selected from 0 to 6; s is an integer selected from 10 to 40; t is an integer selected from 10 to 30; and y is an integer selected from 0 to 3. In some optional embodiments, s is an integer selected from 10 to 25. In some optional embodiments, t is an integer selected from 10 to 16.
(N_{L2})_{y} is an overhang nucleotide that does not undergo base complementary pairing with a nucleotide in the antisense strand.

Each nucleotide N_{L} is independently selected from unmodified nucleotides or from following modified nucleotides: abasic nucleotides, reverse abasic nucleotides, reverse deoxyribonucleotides, 2′-halogen modified nucleotides, 2′-O-methoxyalkyl modified nucleotides, 2′-O-alkyl modified nucleotides, 2′-O-allyl modified nucleotides, 5′-vinyl modified nucleotides, 5′-cyclopropyl modified nucleotides, BNA, LNA or 2′-deoxy modified nucleotides.

[N_{L}(N_{M})]₈ is eight contiguous nucleotides composed of the nucleotide N_{L} and the nucleotide N_{M} located at sites 2-9 of the antisense strand from the 5′-end, and contains at least one nucleotide N_{M} therein. The nucleotide N_{M} is located at any position of sites 2-9 of the antisense strand from the 5′-end; and each nucleotide N_{M} is independently selected from nucleotides represented by the above Formula (I), Formula (II) or Formula (III).

Each nucleotide N_{A} is independently an unmodified nucleotide or selected from following modified nucleotides: abasic nucleotides, reverse abasic nucleotides, reverse deoxyribonucleotides, 2′-halogen modified nucleotides, 2′-O-methoxyalkyl modified nucleotides, 2′-O-methyl modified nucleotides, 2′-O-allyl modified nucleotides or 2′-deoxy modified nucleotides.

Each nucleotide N_{B} is independently an unmodified nucleotide or selected from following modified nucleotides: abasic nucleotides, reverse abasic nucleotides, reverse deoxyribonucleotides, 2′-halogen modified nucleotides, 2′-O-methoxyalkyl modified nucleotides, 2′-O-methyl modified nucleotides, 2′-O-allyl modified nucleotides or 2′-deoxy modified nucleotides.

The nucleotide Nu is selected from nucleotides defined by the nucleotide N_{L}, or is selected from following nucleotides: 5′-vinylphosphate modified nucleotides, 5′-cyclopropylphosphate modified nucleotides or 5′-alkyl substituent phosphate modified nucleotides.

Each nucleotide N_{L2} is independently an unmodified nucleotide or selected from following modified nucleotides: abasic nucleotides, reverse abasic nucleotides, reverse deoxyribonucleotides, 2′-halogen modified nucleotides, 2′-O-methoxyalkyl modified nucleotides, 2′-O-alkyl modified nucleotides, 2′-O-allyl modified nucleotides, 2′-vinyl modified nucleotides, 2′-cyclopropyl modified nucleotides, BNA, LNA or 2′-deoxy modified nucleotides.

In some optional embodiments, when the nucleotide N_{L} is a modified nucleotide, each nucleotide N_{L} is independently selected from following modified nucleotides: 2′-methoxy modified nucleotides, 2′-fluoro modified nucleotides and 2′-deoxy modified nucleotides; and particularly when the nucleotide N_{L} is a nucleotide N_{L} in the [N_{L}(N_{M})]₈, each nucleotide N_{L} is independently preferably a 2′-methoxy modified nucleotide, a 2′-fluoro modified nucleotide or a 2′-deoxy modified nucleotide.

The double-stranded oligonucleotide as represented by Formula (A) satisfies the general formula composed of the above A1 and A2, and also has at least one of following characteristics (i)-(v):
(i) a melting temperature Tm value is not significantly reduced, wherein the melting temperature Tm value being not significantly reduced means that the Tm value is increased or unchanged, or is reduced by no more than 10% (as compared with a parental oligomeric double-stranded nucleotide without modified nucleotide N_{M});
(ii) the sense strand and the antisense strand each independently contain no more than four 2′-halogen modified nucleotides. In some optional embodiments, the sense strand and the antisense strand each independently contain no more than four 2′-fluoro modified nucleotides. In some optional embodiments, the antisense strand contains no more than four 2′-fluoro modified nucleotides; and the sense strand contains no more than three 2′-fluoro modified nucleotides;
(iii) the sense strand and the antisense strand each independently contain 1-4 phosphorothioate internucleotide linkages, for example, including, but not limited to, containing 1, 2, 3 or 4 phosphorothioate internucleotide linkages. In some embodiments, the sense strand and the antisense strand each independently contain at least two phosphorothioate internucleotide linkages;
(iv) the sense strand and the antisense strand each independently contain at least two 2′-methoxy modifications. In some optional embodiments, the sense strand and the antisense strand each independently contain at least three 2′-methoxy modifications. In some optional embodiments, the sense strand and the antisense strand each independently contain at least five 2′-methoxy modifications; and
(v) the double-stranded oligonucleotide contains a duplex region having a length of 17-25 nucleotide pairs, wherein the duplex region may have a length of, but not limited to, for example, 17, 18, 19, 20, 21, 22, 23, 24 or 25 nucleotides. In some optional embodiments, the sense strand and the antisense strand each independently contain 17-23 nucleotides. In some optional embodiments, the sense strand and the antisense strand each independently contain 19-23 nucleotides. In some optional embodiments, the sense strand and the antisense strand each independently contain 19-21 nucleotides.

In some embodiments, in characteristic (ii), the sense strand and the antisense strand each independently contain no more than five 2′-halogen modified nucleotides. In some optional embodiments, the sense strand and the antisense strand each independently contain no more than five 2′-fluoro modified nucleotides. In some optional embodiments, the antisense strand contains no more than five 2′-fluoro modified nucleotides; and the sense strand contains no more than four 2′-fluoro modified nucleotides.

In some optional embodiments, each strand in the dsRNA represented by Formula (A) independently has a length of 17-23 nucleotides; the antisense strand contains at least two phosphorothioate internucleotide linkages; and the sense strand and/or the antisense strand each independently have at least three 2′-methoxy modifications.

In some optional embodiments, each strand in the dsRNA represented by Formula (A) independently has a length of 19-23 nucleotides; and the sense strand and/or the antisense strand each independently have at least five 2′-methoxy modifications.

In some optional embodiments, each strand in the dsRNA represented by Formula (A) independently has a length of 19-21 nucleotides; and the sense strand and/or the antisense strand independently have at least five 2′-methoxy modifications, and each strand independently has no more than four 2′-halogen modifications. Moreover, the dsRNA can be linked with a ligand that can bind to or target hepatocyte or receptor. In some embodiments, the ligand is a multivalent ligand, for example, the ligand may be a GalNAc derivative. In one or more embodiments, the sense strand and antisense strand each independently have no more than five 2′-halogen modifications.

In some optional embodiments, the dsRNA represented by Formula (A) contains one or more overhang regions and/or capping groups at the 3′-end or the 5′-end or both ends of each strand. The overhang may have a length of 1-6 nucleotides. The overhang can mismatch with the target sequence, or it can be complementary to a targeted gene sequence.

In some optional embodiments, in addition to the modified nucleotide N_{M} of the dsRNA represented by the Formula (A), at least two different modifications further may be present on the sense strand and the antisense strand of the dsRNA. In some embodiments, such modifications are combinations of 2′-O-methyl, 2′-fluoro or 2′-deoxy modifications. Besides, in some embodiments, the sense strand and the antisense strand further may independently adopt modification groups other than the 2′-O-methyl modified nucleotide, 2′-deoxy modified nucleotide, and 2′-fluoro modified nucleotide, such as 2′-tert-butyldimethylsilyl nucleotide (TBDMS) modification.

In some optional embodiments, the dsRNA represented by Formula (A) further has at least one of following characteristics: (i) the antisense strand contains no more than four 2′-fluoro modifications, and the sense strand contains no more than three 2′-fluoro modifications; (ii) the antisense strand and the sense strand each independently contain at least two phosphorothioate internucleotide linkages; (iii) the antisense strand and the sense strand each contain no less than five 2′-methoxy modifications; (iv) the antisense strand contains one or more modified nucleotides N_{M}, as represented by any one of Formula (I), Formula (II) or Formula (III) above. In one or more embodiments, the antisense strand contains no more than five 2′-fluoro modifications, and the sense strand contains no more than four 2′-fluoro modifications.

In some optional embodiments, the double-stranded oligonucleotide contains a sense strand as represented by A1b and an antisense strand as represented by A2b, where each strand has a length of 14-30 nucleotides. In this embodiment, the antisense strand contains a sequence complementary to a target gene sequence, and the sense strand contains a sequence sufficiently complementary to an antisense strand sequence so as to form a duplex region. In this embodiment, the double-stranded oligonucleotide is as represented by Formula (β):

5′-(N_{A})ₓ—(N_{L})ₛ—(N_{L2})_{y}-3′; A1b:

3′-(N_{B})_{z}—(N_{L})ₜ—[(N_{L})ᵢ—N_{M}—(N_{L})₇₋ᵢ]₈—N_{L1}-5′ Formula (β), A2b:
where x and z are each independently an integer from 0 to 6, s is an integer from 10 to 25, y is an integer from 0 to 3, t is an integer from 9 to 16, and i is an integer from 0 to 7.

In the above, [(N_{L})ᵢ—N_{M}—(N_{L})₇₋ᵢ]₈ is a form of the above [N_{L}(N_{M})]₈ containing only one nucleotide N_{M}, and thus it has general characteristics of the above [N_{L}(N_{M})]₈. i represents the number of nucleotides N_{L} located at the 3′-end of the nucleotide N_{M} in [(N_{L})ᵢ—N_{M}—(N_{L})₇₋ᵢ]₈, and 7-i represents the number of nucleotides N_{L} located at the 5′-end of the nucleotide N_{M}.

In the above Formula (β), the nucleotide N_{M} has a structure as represented by any one of above Formula (I), Formula (II) or Formula (III); each nucleotide N_{L}, each nucleotide N_{L}1 and each nucleotide N_{L2} are independently selected from unmodified nucleotides or from following modified nucleotides: 2′-deoxy modified nucleotides, abasic nucleotides, 2′-fluoro modified nucleotides and 2′-O methyl modified nucleotides. In the above, each nucleotide N_{A} and each nucleotide N_{B} are independently selected from unmodified nucleotides or from following modified nucleotides: abasic nucleotides, 2′-fluoro modified nucleotides, 2′-O-methyl modified nucleotides or 2′-deoxy modified nucleotides. One or more nucleotides in the sense strand A1b may be complementary to one or more nucleotides in the antisense strand A2b, and as mentioned above, complementation does not occur in (N_{L2})_{y} region.

In some other optional embodiments, the double-stranded oligonucleotide contains a sense strand as represented by A1c and an antisense strand as represented by A2c, where each strand has a length of 17-25 nucleotides. In this embodiment, the antisense strand contains a sequence complementary to a target gene sequence, and the sense strand contains a sequence sufficiently complementary to the antisense strand sequence so as to form a duplex region. In this embodiment, the double-stranded oligonucleotide is as represented by Formula (C):

5′-(N_{A})ₓ—(N_{L})ₛ-3′; A1c:

3′-(N_{B})_{z}—(N_{L})ₜ—[(N_{L})ᵢ—N_{M}—(N_{L})₇₋ᵢ]₈—N_{L1}-5′ A2c: Formula (C),
where x is an integer selected from 0 to 3, s is an integer selected from 14 to 23, z is an integer selected from 1 to 3, t is an integer selected from 10 to 16, and i is an integer selected from 1 to 6; definitions of the nucleotide N_{A}, nucleotide N_{B}, nucleotide N_{L}, nucleotide N_{M} and nucleotide N_{L}1 in Formula (C) are the same as those in Formula (β), respectively. Moreover, at least one nucleotide N_{B} on the antisense strand is linked to a neighboring nucleotide by a phosphorothioate linkage.

In some optional embodiments, the double-stranded oligonucleotide represented by the above Formula (β) or Formula (C) contains a sense strand having a length of 19-21 nucleotides and an antisense strand having a length of 19-23 nucleotides, wherein sequences of the antisense strand and the sense strand are sufficiently complementary to each other so as to form a duplex region of 17-21 base pairs; moreover, the sense strand or the antisense strand each independently has no more than five 2′-fluoro modified nucleotides; and the dsRNA can have a nucleotide overhang at 3′-ends of the sense strand and the antisense strand. For example, there is a nucleotide overhang at the 3′-end of the antisense strand and a blunt end at the 5′-end of the antisense strand.

In some optional embodiments, the dsRNA represented by the above Formula (β) or Formula (C) further has at least one of following characteristics: (i) the antisense strand at least contains one modified nucleotide N_{M} at positions 3-8 counting from the 5′-end; the nucleotide N_{M} is represented by any one selected from the above Formula (I), Formula (II) or Formula (III); (ii) the antisense strand or the sense strand each independently contains 1˜4 phosphorothioate internucleotide linkages, for example, being, but not limited to, 1, 2, 3, or 4 phosphorothioate internucleotide linkages; (iii) the antisense strand or the sense strand of the dsRNA each independently contains at least five 2′-methoxy modifications, and each strand contains no more than four 2′-fluoro modifications; (iv) the sense strand is conjugated to a ligand; (v) the dsRNA contains one or more deoxyribonucleotide modifications; and (v) the dsRNA at least contains one of 2′-O-methyl, 2′-deoxy or 2′-fluoro modifications on each strand. In one or more embodiments, the antisense strand or the sense strand each independently contains no more than five 2′-fluoro modifications.

In some optional embodiments, the dsRNA represented by the above Formula (β) or Formula (C) has one or more or all of following characteristics:
(a) the sense strand has one or more or all of following characteristics: (i) a length of 19-21 nucleotides; (ii) a ligand attached to the 3′-end, the ligand including a GalNAc derivative attached by a branched linker; (iii) no less than five 2′-OMe modifications and no more than four 2′-F modifications, and 0 or one 2′-deoxynucleotide; and, (iv) counting from the 5′-end of the sense strand, at least one phosphorothioate internucleotide linkage between positions 1-3 of nucleotide linking adjacent nucleotides; and in one or more embodiments, the sense strand contains no more than five 2′-F modifications; and
(b) the antisense strand has one or more or all of following characteristics: (i) a length of 21-23 nucleotides, and no less than five 2′-OMe modifications and no more than four 2′-F modifications; (ii) at least one nucleotide N_{M} represented by the above Formula (II) or Formula (III) being present at sites 6-8 counting from the 5′-end; (iii) at least one phosphorothioate internucleotide linkage being present between positions 1-3 of nucleotide, and/or positions 21-23 of nucleotide counting from the 5′-end; and arbitrarily, the dsRNA having two nucleotide overhangs at the 3′-end of the antisense strand and a blunt end at the 5′-end of the antisense strand. In one or more embodiments, the antisense strand contains no more than five 2′-F modifications.

In some optional embodiments, the dsRNA represented by the above Formula (β) or Formula (C) has one or more or all of following characteristics:
(a) the sense strand has one or more or all of following characteristics: (i) a length of 19-21 nucleotides; (ii) containing a ligand attached to the 3′-end, the ligand including a GalNAc derivative attached by a branched linker; (iii) no less than five 2′-OMe modifications and no more than four 2′-F modifications, and 0 or one deoxyribonucleotide or ribonucleotide; and, (iv) counting from the 5′-end, at least one phosphorothioate internucleotide linkage between positions 1-3 of nucleotide linking adjacent nucleotides. In one or more embodiments, the sense strand contains no more than five 2′-F modifications.

In some optional embodiments, positions of 2′-F, 2′-deoxy and other modifications in the sense strand are preferably contrapositions of sites 11, 12, and 13 of the antisense strand.

In some optional embodiments, positions of 2′-F, 2′-deoxy and other modifications in the sense strand are preferably contrapositions of sites 10, 11, 12, and 13 of the antisense strand.
(b) The antisense strand has one or more or all of following characteristics: (i) a length of 21-23 nucleotides, and no less than five 2′-OMe modifications and no more than four 2′-F modifications, wherein counting from the 5′-end, the 2′-F modification is preferably located at at least one of site 2, site 6, site 14 and site 16 of the antisense strand; (ii) at least one nucleotide N_{M} represented by the above Formula (II) or Formula (III) being present at sites 5-8 counting from the 5′-end, preferably the nucleotide N_{M} represented by the above Formula (II) or Formula (III) being present at at least one of site 5, site 6 and site 7; (iii) at least one phosphorothioate internucleotide linkage being present between positions 1-3 of nucleotide, and/or positions 21-23 of nucleotide counting from the 5′-end; and arbitrarily, the dsRNA having two nucleotide overhangs at the 3′-end of the antisense strand and one blunt end at the 5′-end of the antisense strand. In one or more embodiments, the antisense strand contains no more than five 2′-F modifications. In one or more embodiments, in item (i), counting from the 5′-end, the 2′-F modification is preferably located at at least one of site 2, site 6, site 9, site 12, site 14 and site 16 of the antisense strand.

It should be noted that, the present disclosure does not limit sequences of the nucleotides in the above Formulas (A), (B) and (C), and in some optional examples, base sequences of the sense strand and the antisense strand of the double-stranded oligomeric nucleotide are as follows, wherein capital letters A, U, G, C, and T respectively represent types of bases of nucleotide:

TABLE 1

| | Sense Strand | Antisense Strand |
|---|---|---|
| No. | (5′ to 3′ direction) | (5′ to 3′ direction) |
| 1 | CCAAGAGCACCAAGAACUA(SEQ ID | UAGUUCUUGGUGCUCUUGGCU(SEQ |
| | NO: 41) | ID NO: 42) |
| 2 | CAGACAGACAAGACCAUCU(SEQ ID | AGAUGGUCUUGUCUGUCUGGA(SEQ |
| | NO: 43) | ID NO: 44) |
| 3 | GUACGUGGACUGGAUUCUG(SEQ ID | CAGAAUCCAGUCCACGUACUU(SEQ |
| | NO: 45) | ID NO: 46) |

According to another aspect of the present disclosure, the present disclosure further provides a pharmaceutical composition containing the above double-stranded oligonucleotide, wherein the pharmaceutical composition contains the double-stranded oligonucleotide of the present disclosure and a pharmaceutically acceptable adjuvant. The pharmaceutical composition contains a therapeutically effective amount of the above double-stranded oligonucleotide, and the pharmaceutical composition is suitable for reducing or inhibiting expression of a specific target gene in a cell of a subject, particularly when overexpression of target gene product in the subject is associated with a kind of/class of diseases, since the above dsRNA can effectively inhibit target gene expression, and has more excellent anti-off-target effect, the dsRNA as a main or auxiliary active substance therein can effectively relieve or treat various relevant diseases.

In some optional embodiments, the pharmaceutical composition is used for preventing, relieving or treating pathological conditions or diseases caused by expression of a specific gene. In some optional embodiments, when the above dsRNA carries a GalNAc ligand, RNA delivery to hepatocytes can be realized, so that expression of a specific target gene in a cell of a subject can be reduced or inhibited, further for studying, treating or relieving relevant diseases.

The pharmaceutically acceptable adjuvant includes, but is not limited to, one or more of a solvent, a solubilizing agent, a co-solvent, an emulsifier, a colorant, a binder, a disintegrant, a filler, a lubricant, a wetting agent, a tonicity modifier, a stabilizer, a glidant, an anti-caking agent, a flavor enhancer, a bacteriostatic agent, a suspending agent, a coating agent, a film-forming agent, a flavoring agent, a tackifier, an anti-adherent, an antioxidant, an antioxidant synergist, a chelating agent, a pH regulator, an adsorbent, a plasticizer, a surfactant, a thickener, an encapsulating agent, a protective agent, a humectant, a softening agent, an absorbent, a diluent, a release regulator, a pressure sensitive adhesive, a hardener, a vacant capsule, a matrix and a drug carrier material.

In some optional embodiments, the pharmaceutical composition contains: (1) a therapeutically effective amount of the dsRNA of Formula A to Formula C of the present disclosure, and (2) a pharmaceutically acceptable carrier or excipient.

In some optional embodiments, each strand of the dsRNA in the pharmaceutical composition has 19-23 nucleotides. In some optional embodiments, the dsRNA in the pharmaceutical composition has at least one of following characteristics: (i) the antisense strand uses a modified nucleotide N_{M} as represented by above Formula (III) at at least one of positions 2-9 counting from the 5′-end; (ii) the antisense strand and the sense strand each independently contain at least two of other modifications selected from 2′-methoxy modifications, 2′-fluoro modifications or 2′-deoxy and (iii) the antisense strand contains 1-6 phosphorothioate internucleotide linkages.

In some optional embodiments, the dsRNA in the pharmaceutical composition contains at least one phosphorothioate internucleotide linkage, and the phosphorothioate internucleotide linkage may be located at the 3′- or 5′-end of the sense strand and/or the antisense strand. In addition, the dsRNA may have a nucleotide overhang at the 3′-end of the antisense strand and have a blunt end at the 5′-end of the antisense strand.

In some optional embodiments, the dsRNA in the pharmaceutical composition is a dsRNA conjugate reagent formed by containing a ligand, so as to promote delivery or uptake of the dsRNA to or by a specific tissue or cell. The ligand may contain galactose, galactosamine, or N-acetyl galactosamine (GalNAc) or derivatives thereof. The ligand may be covalently attached to the 5′- or 3′-end of the sense strand of the dsRNA by a linker.

The present disclosure further provides use of the double-stranded oligonucleotide represented by Formula A to Formula C or the pharmaceutical composition of the present disclosure in the preparation of drugs for treating and/or preventing pathological conditions or diseases caused by expression of a specific gene.

In some optional embodiments, the nucleotide N_{M} of the double-stranded oligonucleotide contained in the double-stranded oligonucleotide or the pharmaceutical composition in the above use is a modified nucleotide having a structure represented by at least one of Formula (I), Formula (II) or Formula (III) above.

According to another aspect of the present disclosure, the present disclosure further provides a method for inhibiting target gene expression, wherein when the method is applied to inhibit target gene expression, the target gene expression can be effectively inhibited, and an off-target effect is low.

This method contains delivering to a subject the dsRNA molecule of the present disclosure by subcutaneous or intravenous administration. In the above, the dsRNA preferably administered employs a novel nucleoside analogue having any structure represented by above Formula (I), Formula (II) or Formula (III) and a modification mode, so as to eliminate or reduce the off-target effect of the dsRNA. In an example, the methods include making a cell or tissue contact the dsRNA of the present disclosure. The cell or tissue may be in vitro or in vivo. The subject may be any animal, such as mammals, including, but not limited to, mammals such as humans, as well as mice, rats, sheep, cattle, dogs, and cats. In some optional embodiments, the mammal is human.

The method for inhibiting target gene expression provided by the present disclosure is optionally for diagnostic and therapeutic purposes, or non-diagnostic and therapeutic purposes.

In embodiments for the non-disease diagnosis and treatment purposes, inhibiting target gene expression can be used in fields such as relevant cell signal pathway research, gene expression research, drug research and development or animal model construction. In embodiments for disease diagnostic and therapeutic purposes, various diseases are treated by methods of inhibiting target gene expression by administering these dsRNA agents. For example, when the above dsRNA carries a GalNAc ligand, RNA delivery to hepatocytes can be realized so as to treat liver-associated diseases.

The technical solutions and beneficial effects of the present disclosure are further described below in conjunction with preferred examples.

Unless otherwise noted, meanings of base composition and modifications described in various examples herein are as follows: capital letters A, U, G, C, and T represent base composition of nucleotides; lowercase letter m means that a nucleotide represented by a preceding letter thereof is a 2′-methoxy modified nucleotide; lowercase letter f means that a nucleotide represented by a preceding letter thereof is a 2′-fluoro modified nucleotide; lowercase letter d means that a nucleotide represented by a preceding letter thereof is a deoxyribonucleotide (i.e., 2′-deoxy or 2′-H modified nucleotide); brackets plus capital letters TBDMS, i.e., (TBDMS), mean that a nucleotide represented by a preceding letter thereof is a 2′-tert-butyldimethylsilylmodified nucleotide; brackets plus capital letters TOM, i.e., (TOM), mean that a nucleotide represented by a preceding letter thereof is a 2′-triisopropylmethoxysilane modified nucleotide; and lowercase letter s means that nucleotides represented by two preceding and following letters thereof are linked by a phosphorothioate linkage. L96 represents a GalNAc conjugated carrier structure represented by Formula (VI). L96 carrier structure is represented below:

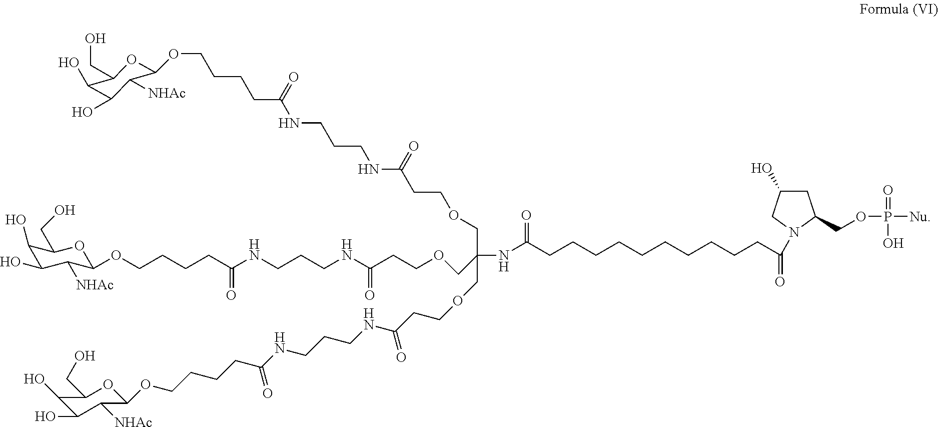

Unless otherwise noted. reagents and consumable materials (TABLE 2) and instrument and equipment (TABLE 3) used in following examples are all products commercially available from following manufacturers.

TABLE 2
Reagents and Consumable Materials

| Name | Manufacturer |
|---|---|
| HepG2 cell | Wuhan Pricella Biotechnology Co., Ltd. |
| HEK293A cell | Wuhan Pricella Biotechnology Co., Ltd. |
| DMEM culture medium | M&C GENE TECHNOLOGY(BEIJING) LTD. |
| Opti-MEM culture medium | GIBCO |
| Fetal bovine serum (FBS) | Merck Ltd |
| Lipofectamine 3000 | Thermo Fisher Scientific |
| Hanwei RNA extraction kit | Zhejiang Hanwei Science and Technology Co. Ltd. |
| Reverse Transcription System | Promega Corporation |
| SYBR Select Master Mix | ABI |
| Deoxyribonuclease I from bovine pancreas | SIGMA |
| Collagenase from Clostridium histolyticum | SIGMA |
| RNALater | Thermo Fisher Scientific |
| Dual-Glo ® Luciferase Assay System | Promega Corporation |

TABLE 3
Main Instrument and Equipment

| Name | Manufacturer |
|---|---|
| Full-automatic nucleic acid extractor | Zhejiang Hanwei Science and Technology Co. |
| | Ltd. |
| High speed refrigerated centrifuge | Eppendorf |
| NANODROP OneC | Thermo Fisher Scientific |
| Gradient PCR amplifier | Eppendorf |
| Fluorescence quantitative PCR instrument | ABI StepOne Plus |
| Gel imager | Shanghai Tanon Science & Technology Co., |
| | Ltd. |
| Electrophoresis apparatus | BEIJING LIUYI INSTRUMENT PLANT |
| CO₂ thermostatic incubator | Thermo Fisher Scientific |
| Biological safety cabinet | Esco Lifesciences |
| Tissuelyser II full-automatic tissue | Shanghai Jingxin Industrial Development Co., |
| homogenizer | Ltd. |
| Synergy H1 microplate reader | Biotek |

Unless otherwise noted, sequences of the dsRNA (siRNA is also used to refer to dsRNA in the following examples) in the following examples were all entrusted to be synthesized by Suzhou Biosyntech Co., Ltd.; plasmid construction and PCR primer synthesis were both entrusted to be completed by Beijing Tsingke Biotech Co., Ltd.; experimental animals C57BL/6J mice and ICR mice were both purchased from SPF (Beijing) Biotechnology Co., Ltd.; and blood biochemical standard measurement was entrusted to be completed by Beijing Sinogenetic Biotechnology Co., Ltd.

Unless otherwise noted, Real-time PCR detection data involved in in vivo/in vitro siRNA activity experiments in the following examples are all used for relative quantitative calculation of gene mRNA of interest in various test groups by a ΔΔCt method, and a calculation method is summarized as follows:

$$Δ Ct ( test group ) = Ct ( gene of interest in test group ) - Ct ( reference gene in test group ) ;$$

$$Δ Ct ( control group ) = Ct ( gene of interest in control group ) - Ct ( reference gene in control group ) ;$$

$$ΔΔ Ct ( test group ) = Δ Ct ( test group ) - Δ Ct ( control group mean ) ;$$

$$ΔΔ Ct ( control group ) = Δ Ct ( control group ) - Δ Ct ( control group mean ) .$$

Taking the control group as reference, expression level of the gene mRNA of interest in the test group is normalized, and remaining expression level of the gene mRNA of interest in the control group is defined as 100%.

$$Relative remaining expression level of gene mRNA of interest in test group = {2}^{- ΔΔ Ct} ( test group ) \times 100 \%$$

$$Inhibitory rate of gene mRNA of interest in test group = 100 \% - relative expression level of gene mRNA of interest in test group .$$

Unless otherwise noted, ratios of the reagents in the following examples are all calculated according to volume ratio (v/v); in vivo/in vitro activity experimental data are all expressed by X+SD, and experimental data are all plotted and analyzed using GraphPad prism 8.0 software.

### Explanations of Terms

The “double-stranded oligonucleotide” as used in the present disclosure refers to a double-stranded structure formed by two oligonucleotides by complementary pairing of part or all of bases thereof, wherein the two oligonucleotides include a sense strand and an antisense strand, whose lengths may be the same or not. Oligonucleotides having a double-stranded structure belong to the double-stranded oligonucleotide of the present disclosure as long as there exists a region where bases are at least partially complementarily paired to form a duplex. The nucleotides forming the double-stranded oligonucleotide in the present disclosure may be modified or unmodified nucleotides, and when the nucleotides are modified nucleotides, unless otherwise noted, the modification in the present disclosure is not specified with a site of modification. For the double-stranded oligonucleotide in the present disclosure, in addition to the nucleotide being modified, a linkage linking nucleotides may also be modified, and double-stranded oligonucleotides containing a modified linkage between nucleotides also belong to the double-stranded oligonucleotide of the present disclosure. In addition to a nucleotide part, the double-stranded oligonucleotide in the present disclosure may also contain a compound molecule or a modifier acceptable in the art, so as to improve properties of the double-stranded oligonucleotide, for example, linked with a ligand to form a conjugate.

The nucleotide in the present disclosure may refer to an independent nucleotide, or a nucleotide residue in an oligonucleotide; the nucleotide in the present disclosure may be singular or plural, and when plural, it can refer to a class of nucleotides. In the present disclosure, the double-stranded oligonucleotide can also be represented by dsRNA, siRNA or double-stranded oligomeric nucleotide, and the double-stranded oligonucleotide, double-stranded oligomeric nucleotide, dsRNA and siRNA herein can be used interchangeably. 2-methoxy in the present disclosure may also be represented by 2′-OMe, and 2-methoxy and 2′-OMe can be used interchangeably in the present disclosure.

The term “conjugate group” used in the present disclosure refers to an atom or atomic group bound to oligonucleotide or other oligomers. Generally, a conjugate group modifies one or more properties of a compound to which it is linked, including but not limited to pharmacodynamics, pharmacokinetics, binding, absorption, cell distribution, cell uptake, charge and/or clearance properties. When linking between two molecules is involved, the term “link” as used herein means that two molecules are directly or indirectly linked by a covalent bond, or that the two molecules are associated by a non-covalent bond (e.g., hydrogen bond or ionic bond).

The term “pharmaceutical composition” or “composition” used in the present disclosure refers to a mixture of substances suitable for administration to individuals. For example, although not intended to limiting, a pharmaceutical composition may contain one or more active agents and pharmaceutically acceptable carriers, also referred to as “pharmaceutically acceptable carriers” (e.g., sterile aqueous solution) herein. In some embodiments, the pharmaceutical composition is sterile.

The terms “optionally substituted” and “possibly substituted or unsubstituted” used in the present disclosure mean that a group, as defined herein, is substituted with zero or more (e.g., one, two or three) substituents independently selected. One non-limiting example of independently selected substituent includes alkyl, amino, (alkyl)amino, (alkyl) carbonyl, (aryl) carbonyl, (alkoxy) carbonyl, [(alkoxy) carbonyl]amino, carboxyl, aryl, heteroaryl, urcido, guanidino, halogen, sulfonamido, hydroxy, (alkyl) sulfanyl, nitro, haloalkoxy, aryloxy, aralkyloxy, (alkyl) sulfonyl, (cycloalkyl) sulfonyl, (aryl) sulfonyl, cycloalkyl, sulfanyl, caboxamido, heterocyclyl and (heterocyclyl) sulfonyl.

The term “mercapto” used in the present disclosure refers to SH group.

In the present disclosure, the term “small interfering RNA (siRNA)” is a double-stranded RNA having a length of 17 to 25 nucleotides, and contains a sense strand and an antisense strand. The siRNA mediates RNA transcript targeted cleavage of RISC pathway by forming a silencing complex (RNA-induced silencing complex, RISC). Specifically, siRNA guides specific degradation of mRNA sequence through a known RNA interference (RNAi) process, and inhibits translation of mRNA into amino acids and conversion to proteins.

Conjugate groups used in the present disclosure refers to target ligands, at least one of the target ligand is capable of binding to one or more cell receptors, cell channels and/or cell transporters capable of promoting endocytosis. In some embodiments, at least one conjugate group may contain at least one of N-acetylgalactosamine (GalNAc), galactose, galactosamine, N-formylgalactosamine, N-propionylgalactosamine, N-butanoylgalactosamine, N-isobutanoylgalactosamine, macrocycles, folic acid molecules, fatty acids, bile acids, cholesterol and derivatives thereof.

In some embodiments, at least one of the conjugate groups of the present disclosure is capable of binding to at least one asialoglycoprotein receptor (ASGPR), where ASGPR is a lectin located on hepatocytes, which can bind to galactose residues.

In some embodiments, all ribonucleotides can be modified for the double-stranded siRNA. In some embodiments, at least one strand of the double-stranded siRNA may contain at least one phosphorothioate linkage. In some embodiments, at least one strand of the double-stranded siRNA may contain up to six phosphorothioate linkages. In some embodiments, the oligonucleotide may contain siRNA containing one or more phosphorothioate backbone linkages. Those skilled in the art know that the modified nucleotides in the present disclosure contained at certain positions of the antisense strand of siRNA are resistant to activity and can promote reduction of off-target activity.

The double-stranded oligonucleotide conjugate of the present disclosure or the composition thereof may be administered in formulation, the formulation may be administered in a pharmaceutically acceptable solution, and the solution may conventionally contain salt, buffer, preservative, compatible carrier, adjuvant and optionally other therapeutic ingredients at pharmaceutically acceptable concentrations. The method according to the present disclosure may be applied with a pharmaceutical composition. Generally, the pharmaceutical composition contains the double-stranded oligonucleotide conjugate of the present disclosure and a pharmaceutically acceptable carrier. Pharmaceutically acceptable carriers are well known to those skilled in the art and can be selected and used by conventional methods. The pharmaceutically acceptable carrier as used herein means a non-toxic material that does not interfere with effectiveness of biological activity of active ingredients.

### Example 1

Synthesis of siRNA:

Synthesis of siRNA was not different from a usual phosphoramidite solid-phase synthesis method. When a nucleotide modified with a bulky group needed to be introduced when synthesizing an antisense strand containing a bulky group such as TBDMS, TOM, and TIPS, the above phosphoramidite monomer was substituted with a nucleotide monomer at corresponding site of an original parental sequence.

siRNA sequence (siRNA single strand) to be synthesized was introduced to ABI394 synthesizer, and synthesis was performed according to a set synthesis program with Universal CPG/PS as carrier. Introduction of each base needed to undergo four steps of reactions of removing DMTr, coupling, oxidation and capping. Nucleoside phosphoramidite monomers modified with 2′-TBDMS, 2′-OMe, 2′-F and 2′-TOM were all purchased from Shanghai Hongene Biotech Corporation. 5-ethylmercapto-1H-tetrazole (ETT) was used as activator (0.6 M acetonitrile solution), 0.2 M xanthane hydride dissolved in acetonitrile and pyridine in a volume ratio of 1:1 (Suzhou Kroma) was used as thionating reagent, and 0.05 M iodine dissolved in pyridine and water solution in a volume ratio of 9:1 (Suzhou Kroma) was used as oxidant.

After synthesis was ended, the carrier was unloaded and blow-dried, deprotected with strong ammonia water (50° C., 16 h), and cooled in −20° C. refrigerator for 10 min after the reaction was ended. After centrifugation, supernatant was transferred into another centrifugation tube, concentrated to driness, and then purified by a RP-HPLC method, wherein mobile phase A was 0.1 M TEAA, and mobile phase B was acetonitrile. HPLC, MS and UV detections were performed on purification-collected fractions, and qualified fractions were collected and then lyophilized to render siRNA single strands. The obtained siRNA single strands were annealed, to finally render siRNA double strands.

Sequences and modification information of siRNA compounds involved in the following examples are shown in TABLE 4.

TABLE 4
Sequences and Modification Information of siRNA Compounds

| Compound | | Sense Strand/Antisense Strand | |
|---|---|---|---|
| No | Strand | Sequence and Modification (5′-3′) | Sequence ID |
| RX000002 | Sense | UmsUmsCmUmCmCmGfAdAfCmGmUmGmUmCm | SEQ ID NO: 1 |
| | | AmCmGmUm | |
| | Antisense | AmsCfsGmUmGmAfCmAmCmGmUmUmCmGfGm | SEQ ID NO: 5 |
| | | AfGmAmAmsCmsUm | |
| RX502001 | Sense | CmsAmsGmAmCmAmGfAdCfAmAmGmAmCmCm | SEQ ID NO: 2 |
| | | AmUmCmUm | |
| | Antisense | AmsGfsAmUmGmGfUmCmUmUmGmUmCmUfGm | SEQ ID NO: 6 |
| | | UfCmUmGmsGmsAm | |
| RX502002 | Sense | CmsAmsGmAmCmAmGfAdCfAmAmGmAmCmCm | SEQ ID NO: 2 |
| | | AmUmCmUm | |
| | Antisense | A(TBDMS)sGfsAmUmGmGfUmCmUmUmGmUmC | SEQ ID NO: 7 |
| | | mUfGmUfCmUmGmsGmsAm | |
| RX502003 | Sense | CmsAmsGmAmCmAmGfAdCfAmAmGmAmCmCm | SEQ ID NO: 2 |
| | | AmUmCmUm | |
| | Antisense | AmsG(TBDMS)sAmUmGmGfUmCmUmUmGmUm | SEQ ID NO: 8 |
| | | CmUfGmUfCmUmGmsGmsAm | |
| RX502004 | Sense | CmsAmsGmAmCmAmGfAdCfAmAmGmAmCmCm | SEQ ID NO: 2 |
| | | AmUmCmUm | |
| | Antisense | AmsGfsA(TBDMS)UmGmGfUmCmUmUmGmUmC | SEQ ID NO: 9 |
| | | mUfGmUfCmUmGmsGmsAm | |
| RX502005 | Sense | CmsAmsGmAmCmAmGfAdCfAmAmGmAmCmCm | SEQ ID NO: 2 |
| | | AmUmCmUm | |
| | Antisense | AmsGfsAmU(TBDMS)GmGfUmCmUmUmGmUmC | SEQ ID NO: 10 |
| | | mUfGmUfCmUmGmsGmsAm | |
| RX502006 | Sense | CmsAmsGmAmCmAmGfAdCfAmAmGmAmCmCm | SEQ ID NO: 2 |
| | | AmUmCmUm | |
| | Antisense | AmsGfsAmUmG(TBDMS)GfUmCmUmUmGmUmC | SEQ ID NO: 11 |
| | | mUfGmUfCmUmGmsGmsAm | |
| RX502007 | Sense | CmsAmsGmAmCmAmGfAdCfAmAmGmAmCmCm | SEQ ID NO: 2 |
| | | AmUmCmUm | |
| | Antisense | AmsGfsAmUmGmG(TBDMS)UmCmUmUmGmUm | SEQ ID NO: 12 |
| | | CmUfGmUfCmUmGmsGmsAm | |
| RX502008 | Sense | CmsAmsGmAmCmAmGfAdCfAmAmGmAmCmCm | SEQ ID NO: 2 |
| | | AmUmCmUm | |
| | Antisense | AmsGfsAmUmGmGfU(TBDMS)CmUmUmGmUmC | SEQ ID NO: 13 |
| | | mUfGmUfCmUmGmsGmsAm | |
| RX502009 | Sense | CmsAmsGmAmCmAmGfAdCfAmAmGmAmCmCm | SEQ ID NO: 2 |
| | | AmUmCmUm | |
| | Antisense | AmsGfsAmUmGmGfUmC(TBDMS)UmUmGmUmC | SEQ ID NO: 14 |
| | | mUfGmUfCmUmGmsGmsAm | |
| RX502010 | Sense | CmsAmsGmAmCmAmGfAdCfAmAmGmAmCmCm | SEQ ID NO: 2 |
| | | AmUmCmUm | |
| | Antisense | AmsGfsAmUmGmGfUmCmU(TBDMS)UmGmUmC | SEQ ID NO: 15 |
| | | mUfGmUfCmUmGmsGmsAm | |
| RZ597024 | Sense | CmsCmsAmAmGmAmGfCdAfCmCmAmAmGmAm | SEQ ID NO: 3 |
| | | AmCmUmAm_L96 | |
| | Antisense | UmsAfsGmUmUmCfUmUmGmGmUmGmCmUfCm | SEQ ID NO: 16 |
| | | UfUmGmGmsCmsUm | |
| RZ597025 | Sense | CmsCmsAmAmGmAmGfCdAfCmCmAmAmGmAm | SEQ ID NO: 3 |
| | | AmCmUmAm_L96 | |
| | Antisense | UmsAfsG(TBDMS)UmUmCfUmUmGmGmUmGmC | SEQ ID NO: 17 |
| | | mUfCmUfUmGmGmsCmsUm | |
| RZ597026 | Sense | CmsCmsAmAmGmAmGfCdAfCmCmAmAmGmAm | SEQ ID NO: 3 |
| | | AmCmUmAm_L96 | |
| | Antisense | UmsAfsGmU(TBDMS)UmCfUmUmGmGmUmGmC | SEQ ID NO: 18 |
| | | mUfCmUfUmGmGmsCmsUm | |
| RZ597027 | Sense | CmsCmsAmAmGmAmGfCdAfCmCmAmAmGmAm | SEQ ID NO: 3 |
| | | AmCmUmAm_L96 | |
| | Antisense | UmsAfsGmUmU(TBDMS)CfUmUmGmGmUmGmC | SEQ ID NO: 19 |
| | | mUfCmUfUmGmGmsCmsUm | |
| RZ597028 | Sense | CmsCmsAmAmGmAmGfCdAfCmCmAmAmGmAm | SEQ ID NO: 3 |
| | | AmCmUmAm_L96 | |
| | Antisense | UmsAfsGmUmUmC(TBDMS)UmUmGmGmUmGm | SEQ ID NO: 20 |
| | | CmUfCmUfUmGmGmsCmsUm | |
| RZ597029 | Sense | CmsCmsAmAmGmAmGfCdAfCmCmAmAmGmAm | SEQ ID NO: 3 |
| | | AmCmUmAm_L96 | |
| | Antisense | UmsAfsGmUmUmCfU(TBDMS)UmGmGmUmGmC | SEQ ID NO: 21 |
| | | mUfCmUfUmGmGmsCmsUm | |
| RZ597030 | Sense | CmsCmsAmAmGmAmGfCdAfCmCmAmAmGmAm | SEQ ID NO: 3 |
| | | AmCmUmAm_L96 | |
| | Antisense | UmsAfsGmUmUmCfUmU(TBDMS)GmGmUmGmC | SEQ ID NO: 22 |
| | | mUfCmUfUmGmGmsCmsUm | |
| RZ597031 | Sense | CmsCmsAmAmGmAmGfCdAfCmCmAmAmGmAm | SEQ ID NO: 3 |
| | | AmCmUmAm_L96 | |
| | Antisense | UmsAfsGmUmUmCfUmUmG(TBDMS)GmUmGmC | SEQ ID NO: 23 |
| | | mUfCmUfUmGmGmsCmsUm | |
| RZ594001 | Sense | GmsUmsAmCmGmUmGfGdAfCmUmGmGmAmUm | SEQ ID NO: 4 |
| | | UmCmUmGm_L96 | |
| | Antisense | CmsAfsGmAmAmUfCmCmAmGmUmCmCmAfCm | SEQ ID NO: 24 |
| | | GfUmAmCmsUmsUm | |
| RZ594002 | Sense | GmsUmsAmCmGmUmGfGdAfCmUmGmGmAmUm | SEQ ID NO: 4 |
| | | UmCmUmGm_L96 | |
| | Antisense | CmsAfsGmAmA(TOM)UfCmCmAmGmUmCmCmA | SEQ ID NO: 25 |
| | | fCmGfUmAmCmsUmsUm | |
| RZ594003 | Sense | GmsUmsAmCmGmUmGfGdAfCmUmGmGmAmUm | SEQ ID NO: 4 |
| | | UmCmUmGm_L96 | |
| | Antisense | CmsAfsGmAmAmU(TOM)CmCmAmGmUmCmCm | SEQ ID NO: 26 |
| | | AfCmGfUmAmCmsUmsUm | |
| RZ594004 | Sense | GmsUmsAmCmGmUmGfGdAfCmUmGmGmAmUm | SEQ ID NO: 4 |
| | | UmCmUmGm_L96 | |
| | Antisense | CmsAfsGmAmAmUfC(TOM)CmAmGmUmCmCmA | SEQ ID NO: 27 |
| | | fCmGfUmAmCmsUmsUm | |
| RZ594005 | Sense | GmsUmsAmCmGmUmGfGdAfCmUmGmGmAmUm | SEQ ID NO: 4 |
| | | UmCmUmGm_L96 | |
| | Antisense | CmsAfsGmAmAmUfCmC(TOM)AmGmUmCmCmA | SEQ ID NO: 28 |
| | | fCmGfUmAmCmsUmsUm | |
| RZ594006 | Sense | GmsUmsAmCmGmUmGfGdAfCmUmGmGmAmUm | SEQ ID NO: 4 |
| | | UmCmUmGm_L96 | |
| | Antisense | CmsAfsGmAmAmUfCmCmA(TOM)GmUmCmCmA | SEQ ID NO: 29 |
| | | fCmGfUmAmCmsUmsUm | |
| RZ002001 | Sense | CmsAmsGmAmCmAmGfAdCfAmAmGmAmCmCm | SEQ ID NO: 2 |
| | | AmUmCmUm_L96 | |
| | Antisense | AmsGfsAmUmGmGfUmCmUmUmGmUmCmUfGm | SEQ ID NO: 30 |
| | | UfCmUmGmsGmsAm | |
| RZ502014 | Sense | CmsAmsGmAmCmAmGfAdCfAmAmGmAmCmCm | SEQ ID NO: 57 |
| | | AmUmCmUm_L96 | |
| | Antisense | AmsGfsAmUmGmGfU(TOM)CmUmUmGmUmCm | SEQ ID NO: 31 |
| | | UfGmUfCmUmGmsGmsAm | |
| RZ502015 | Sense | CmsAmsGmAmCmAmGfAdCfAmAmGmAmCmCm | SEQ ID NO: 57 |
| | | AmUmCmUm_L96 | |
| | Antisense | AmsGfsAmUmGmGfUmCmU(TOM)UmGmUmCm | SEQ ID NO: 32 |
| | | UfGmUfCmUmGmsGmsAm | |
| RX597001 | Sense | CmsCmsAmAmGmAmGfCdAfCmCmAmAmGmAm | SEQ ID NO: 3 |
| | | AmCmUmAm | |
| | Antisense | UmsAfsGmUmUmCfUmUmGmGmUmGmCmUfCm | SEQ ID NO: 33 |
| | | UfUmGmGmsCmsUm | |
| RX597002 | Sense | CmsCmsAmAmGmAmGfCdAfCmCmAmAmGmAm | SEQ ID NO: 58 |
| | | AmCmUmAm | |
| | Antisense | UmsAfsG(TBDMS)UmUmCfUmUmGmGmUmGmC | SEQ ID NO: 34 |
| | | mUfCmUfUmGmGmsCmsUm | |
| RX597003 | Sense | CmsCmsAmAmGmAmGfCdAfCmCmAmAmGmAm | SEQ ID NO: 58 |
| | | AmCmUmAm | |
| | Antisense | UmsAfsGmU(TBDMS)UmCfUmUmGmGmUmGmC | SEQ ID NO: 35 |
| | | mUfCmUfUmGmGmsCmsUm | |
| RX597004 | Sense | CmsCmsAmAmGmAmGfCdAfCmCmAmAmGmAm | SEQ ID NO: 58 |
| | | AmCmUmAm | |
| | Antisense | UmsAfsGmUmU(TBDMS)CfUmUmGmGmUmGmC | SEQ ID NO: 36 |
| | | mUfCmUfUmGmGmsCmsUm | |
| RX597005 | Sense | CmsCmsAmAmGmAmGfCdAfCmCmAmAmGmAm | SEQ ID NO: 58 |
| | | AmCmUmAm | |
| | Antisense | UmsAfsGmUmUmC(TBDMS)UmUmGmGmUmGm | SEQ ID NO: 37 |
| | | CmUfCmUfUmGmGmsCmsUm | |
| RX597006 | Sense | CmsCmsAmAmGmAmGfCdAfCmCmAmAmGmAm | SEQ ID NO: 58 |
| | | AmCmUmAm | |
| | Antisense | UmsAfsGmUmUmCfU(TBDMS)UmGmGmUmGmC | SEQ ID NO: 38 |
| | | mUfCmUfUmGmGmsCmsUm | |
| RX597007 | Sense | CmsCmsAmAmGmAmGfCdAfCmCmAmAmGmAm | SEQ ID NO: 58 |
| | | AmCmUmAm | |
| | Antisense | UmsAfsGmUmUmCfUmU(TBDMS)GmGmUmGmC | SEQ ID NO: 39 |
| | | mUfCmUfUmGmGmsCmsUm | |
| RX597008 | Sense | CmsCmsAmAmGmAmGfCdAfCmCmAmAmGmAm | SEQ ID NO: 58 |
| | | AmCmUmAm | |
| | Antisense | UmsAfsGmUmUmCfUmUmG(TBDMS)GmUmGmC | SEQ ID NO: 40 |
| | | mUfCmUfUmGmGmsCmsUm | |
| RZ000002 | Sense | UmsUmsCmUmCmCmGfAdAfCmGmUmGmUmCm | SEQ ID NO: 59 |
| | | AmCmGmUm_L96 | |
| | Antisense | AmsCfsGmUmGmAfCmAmCmGmUmUmCmGfGm | SEQ ID NO: 5 |
| | | AfGmAmAmsCmsUm | |

### Example 2

In Vitro Inhibitory Activity of siRNAs with TBDMS Modification at Different Sites of Antisense Strand on Complement Component 3 (CC3) mRNA in HepG2 Cells.

The present example evaluated inhibitory activity of compounds RX502002, RX502003, RX502004, RX502005, RX502006, RX502007, RX502008, RX502009, and RX502010 with TBDMS modification at different sites of antisense strand, control compound RX502001 without TBDMS modification, and negative control compound RX000002 on target gene CC3 of interest in HepG2 cells by an in vitro cell screening method.

2.1 Formulation of tested articles: each siRNA tested article above was centrifuged, and then dissolved by adding a suitable amount of 1×PBS according to specification of each vial, to be formulated into 20 μM mother liquor, which was then further diluted with 1×PBS into 5 μM working solution.

### 2.2 Cell Culturing and Plating:

HepG2 cells were cultured and proliferated in DMEM culture medium containing 10% FBS in a 37° C., 5% CO₂ incubator. Before plating, the culture medium was discarded, and rinsing was performed with 0.25% pancreatin. After the cells were digested by pancreatin, digestion of the culture medium was terminated. The cells were resuspended, centrifuged at 800 rpm for 5 min, resuspended with fresh DMEM culture medium containing 10% FBS, counted, and diluted to cell density of 1×10⁵ cells/mL, thoroughly mixed, plated in a 24-well plate, 500 μL/well, and subjected to a transfection operation after 24 h of culturing.

### 2.3 Cell Transfection

2.3.1 Formulation of solution 1-siRNA mixed solution: uniformly mixing 49.4 μL of Opti-MEM and 0.6 μL of siRNA test substance working solution (5 μM) per cell well. Each siRNA tested article was prepared with 3 cell replicate wells.

2.3.2 Formulation of solution 2-Lipofectamine 3000 mixed solution: gently mixing transfection reagent upside down, diluting 2 μL of Lipo3000 according to 48 μL of Opti-MEM per cell well, gently pipetting 3-5 times, uniformly mixing, and standing for 5 min. Each siRNA tested article was prepared with 3 cell replicate wells.

2.3.3 Formulation of solution 3-mixture of solution 1 and solution 2: gently mixing one part (50 μL) of the solution 1 with one part (50 μL) of the solution 2 to formulate solution 3, (100 μL) transfection complex, and incubating at room temperature for 20 min.

2.3.4 A cell culture plate to be transfected was taken out, original culture medium was discarded, and 500 μL of Opti-MEM was added. The solution 3 (100 μL) having undergone the incubation was added dropwise into cell culture wells to render concentration of about 5 nM of the siRNA test substance in each cell transfection well. The cell culture plate was further cultured in the 37° C., 5% CO₂ incubator for 4 h, and 1 mL of DMEM culture medium containing 20% FBS was supplemented to each well. Further culturing was carried out in the 37° C., 5% CO₂ incubator for 24 h.

HepG2 cells were normally cultured without any transfection procedure in Blank control group; and Mock control group was a control group in which no siRNA was added to the transfection complex, and only Lipofectamine 3000 transfection reagent was added.

### 2.4 Detection:

RNA extraction: total RNAs in cells of various wells were extracted using full-automatic nucleic acid extraction instrument and a nucleic acid extraction kit from Zhejiang Hanwei Science and Technology Co. Ltd. according to a method described in instructions.

Reverse transcription reaction: 1 μg of total RNAs extracted from cells in each well were taken, and configured into 20 μL of reverse transcription system according to a method described in kit instructions by using a reverse transcription kit (Reverse Transcription System, A3500) from Promega company and selecting Oligo (dT) 15 reverse transcription primer, and the reverse transcription reaction was completed. After the reaction was ended, 80 μL of RNase-Free water was added into the reverse transcription system to render cDNA solution for Real-time PCR detection.

Real-time PCR detection: 20 μL of Real-time PCR reaction system per PCR detection well was configured according to a method described in kit instructions by using SYBR™ Select Master Mix (Catalog number: 4472908) reagent from ABI company. Each detection system contained 5 μL of cDNA template obtained from the above reverse transcription reaction, 10 μL of SYBR™ Select Master Mix, 0.5 μL of 10 μM upstream primers, 0.5 μL of 10 μM downstream primers (see TABLE 5 for primer information), and 4 μL of RNase-Free H₂O. The configured reaction system was subjected to Real-time PCR amplification on ABI StepOnePlus PCR instrument by a three-step method, wherein an amplification procedure was pre-denaturation at 95° C. for 10 min, then denaturation at 95° C. for 30 s, annealing at 60° C. for 30 s, and extension at 72° C. for 30 s, and processes of denaturation, annealing and extension were repeated for 40 cycles. Gene expression differences were calculated by the above ΔΔCt method after completion of the procedure.

TABLE 5
Sequence Information

| Target Point | Primer Type | Primer Sequence (5′-3′) | Sequence ID |
|---|---|---|---|
| CC3 | Upstream Primer | AAGCGCATTCCGATTGAGGA | SEQ ID NO: 47 |
| | Downstream Primer | CCTGAGTGCAAGATGACGGT | SEQ ID NO: 48 |
| GAPDH | Upstream Primer | GGTCGGAGTCAACGGATTT | SEQ ID NO: 49 |
| | Downstream Primer | CCAGCATCGCCCCACTTGA | SEQ ID NO: 50 |

### 2.5 Analysis:

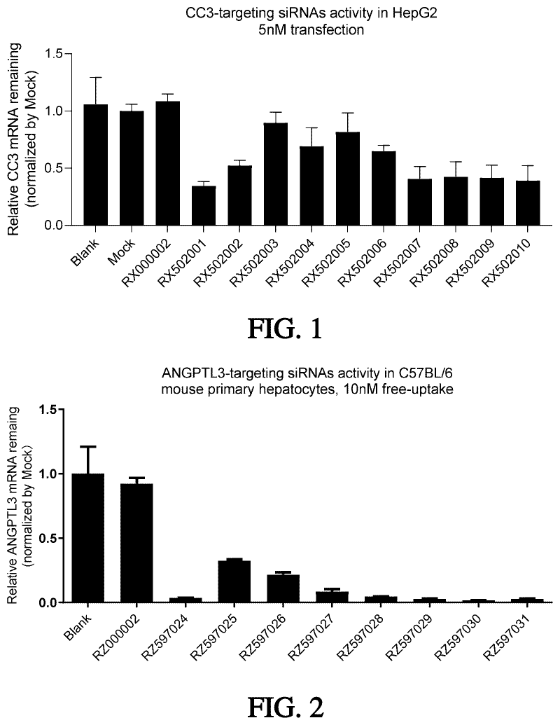

As can be seen from results in Example 2, effects of TBDMS modifications at different sites of the siRNA antisense strand on in vitro cell activity of the siRNA have certain differences. The TBDMS modifications at site 6 (RX502007), site 7 (RX502008), site 8 (RX502009), and site 9 (RX502010) of the antisense strand have comparable activity to the control sequence (RX502001) without TBDMS modification (FIG. 1, TABLE 6).

TABLE 6
Inhibitory Activity of HepG2 on Target Gene
CC3 mRNA after Transfection with Various Test
Substances Tested in the Present Example

| Group | Mean Value of Inhibitory Rate | SD Value |
|---|---|---|
| Blank | −5.78% | ±0.2363 |
| Mock | 0.00% | ±0.0605 |
| RX000002 | −8.37% | ±0.0643 |
| RX502001 | 65.64% | ±0.0394 |
| RX502002 | 47.78% | ±0.0477 |
| RX502003 | 10.39% | ±0.0929 |
| RX502004 | 30.85% | ±0.1613 |
| RX502005 | 18.25% | ±0.1656 |
| RX502006 | 35.20% | ±0.0516 |
| RX502007 | 59.27% | ±0.1073 |
| RX502008 | 57.67% | ±0.1325 |
| RX502009 | 58.46% | ±0.1111 |
| RX502010 | 60.98% | ±0.1312 |

### Example 3

In Vitro Inhibitory Activity of siRNA Conjugates with TBDMS Modification at Different Sites of Antisense Strand on Angiopoietin-Like 3 (ANGPTL3) mRNA in Mouse Primary Hepatocytes

The present example evaluated inhibitory activity of siRNA conjugates RZ597025, RZ597026, RZ597027, RZ597028, RZ597029, RZ597030, and RZ597031 with TBDMS modification at different sites of antisense strand, control conjugate RZ597024 without TBDMS modification, and negative control conjugate RZ000002 targeting no gene on target gene ANGPTL3 of interest in mouse primary hepatocytes by an in vitro mouse primary hepatocyte screening method.

### 3.1 Formulation of Tested Articles:

Each siRNA conjugate tested article above was centrifuged, and then dissolved by adding a suitable amount of 1×PBS according to specification of each vial, and formulated into 20 μM mother liquor, which was then further diluted with 1×PBS into 5 μM working solution.

### 3.2 Isolation of Mouse Primary Hepatocytes:

(1) Anesthesia: C57BL/6j mice were intraperitoneally injected with 0.04 mL/10 g 10% chloral hydrate solution.
(2) Perfusion: animals were fixed and disinfected with 75% ethanol for abdomen and chest thereof. Abdomen was opened to find hepatic portal veins and inferior vena cavas. Indwelling needle was introduced from a lower end of the inferior vena cavas, and HBSS solution started to be perfused at a rate of 120 drops/min. The hepatic portal veins were cut, to confirm that perfusate flowed out from the cut hepatic portal veins. Infusion apparatus was well fixed, and perfusion was continued for 4 min.
(3) Digestion of hepatocytes: the HBSS solution was changed into HBSS containing 0.08% type IV collagenase (Collagenase from Clostridium histolyticum, Sigma), which was perfused further for 8 min at a rate of 120 drops/min.
(4) Isolation of primary hepatocytes: perfused livers were removed from the animals and shredded in sterile petri dish containing 10 mL DMEM. Undigested hepatic tissues and connective tissues were filtered and removed by cytoscreener (75 μm). Cells were centrifuged, and resuspended and washed twice with DMEM. 10 mL of DMEM culture medium containing 10% FBS was added for resuspension, and counting, to wait for an operation of drug free uptake.
   3.3 Free Uptake of siRNA Conjugates:

Freshly isolated mouse primary hepatocytes were plated into type I collagen-coated 12-well cell culture plate (Corning Incorporated), containing 5×10⁵ viable cells and 2 mL of DMEM culture medium containing 10% FBS in each well.

4 μL of the above 5 μM siRNA conjugate working solution was added into each cell well, and gently mixed to render a final concentration of 10 nM of the siRNA conjugate in each cell well. Blank control group was original mouse primary hepatocytes without any operation of free uptake of siRNA conjugate. Mouse primary hepatocytes having undergone free uptake treatment were further cultured in a 37° C., 5% CO₂ incubator for 24 h.

### 3.4 Detection:

RNA extraction: total RNAs in cells of various wells were extracted using full-automatic nucleic acid extraction instrument and a nucleic acid extraction kit from Zhejiang Hanwei Science and Technology Co. Ltd. according to a method described in instructions.

Reverse transcription reaction: 1 μg of total RNAs extracted from cells in each well was taken, and configured into 20 μL of reverse transcription system according to a method described in kit instructions by using a reverse transcription kit (Reverse Transcription System, A3500) from Promega company and selecting Oligo (dT) 15 reverse transcription primer, and the reverse transcription reaction was completed. After the reaction was ended, 80 μL of RNase-Free water was added into the reverse transcription system to render cDNA solution for Real-time PCR detection.

Real-time PCR detection: 20 μL of Real-time PCR reaction system per PCR detection well was configured according to a method described in kit instructions by using SYBR™ Select Master Mix (Catalog number: 4472908) from ABI company. Each detection system contained 5 μL of cDNA template obtained from the above reverse transcription reaction, 10 μL of SYBR™ Select Master Mix, 0.5 μL of 10 μM upstream primers, 0.5 μL of 10 μM downstream primers (see TABLE 7 for primer information), and 4 μL of RNase-Free H₂O. The configured reaction system was subjected to Real-time PCR amplification on ABI StepOnePlus PCR instrument by a three-step method, wherein an amplification procedure was pre-denaturation at 95° C. for 10 min, then denaturation at 95° C. for 30 s, annealing at 60° C. for 30 s, and extension at 72° C. for 30 s, and processes of denaturation, annealing and extension were repeated for 40 cycles. Gene expression differences were calculated by the above ΔΔCt method after completion of the procedure.

TABLE 7
Primer Sequence Table

| Target Point | Primer Type | Primer Sequence (5′-3′) | Sequence ID |
|---|---|---|---|
| ANGPTL3 | Upstream Primer | GAGGAGCAGCTAACCAACTTAAT | SEQ ID NO: 51 |
| | Downstream Primer | TCTGCATGTGCTGTTGACTTAAT | SEQ ID NO: 52 |
| GAPDH | Upstream Primer | TGCACCACCAACTGCTTAG | SEQ ID NO: 53 |
| | Downstream Primer | GGATGCAGGGATGATGTTC | SEQ ID NO: 54 |

### 3.5 Analysis:

As can be seen from results in Example 3, site 5 (RZ597027), site 6 (RZ597028), site 7 (RZ597029), site 8 (RZ597030), and site 9 (RZ597031) of the siRNA antisense strand have good tolerance to TBDMS modification, RZ597027 and RZ597028 have substantially comparable in vitro activity to the control sequence (RZ597024) without TBDMS modification, and RZ597029, RZ597030, and RZ597031 even have in vitro activity superior to the control sequence (RZ597024) without TBDMS modification (FIG. 2, TABLE 8).

TABLE 8 shows the inhibitory activity of mouse primary hepatocytes on the target gene ANGPTL3 mRNA after free uptake of various test substances tested in the present example.

| Group | Mean Value of Inhibitory Rate | SD Value |
|---|---|---|
| Blank | 0.00% | ±0.2097 |
| RZ000002 | 7.73% | ±0.0461 |
| RZ597024 | 96.49% | ±0.0025 |
| RZ597025 | 67.42% | ±0.0097 |
| RZ597026 | 78.32% | ±0.0191 |
| RZ597027 | 91.63% | ±0.0194 |
| RZ597028 | 95.48% | ±0.0027 |
| RZ597029 | 97.08% | ±0.0019 |
| RZ597030 | 98.35% | ±0.0032 |
| RZ597031 | 97.21% | ±0.0032 |

### Example 4

In Vitro Inhibitory Activity of siRNA Conjugates with TOM Modification at Different Sites of Antisense Strand on Coagulation Factor XI (FXI) mRNA in Mouse Primary Hepatocytes

The inhibitory activity of the siRNA conjugates with TOM modification at different sites of antisense strand on the target gene FXI of interest in mouse primary hepatocytes was evaluated by the same experimental method as that in Example 3. Differences lie in that the siRNA conjugates RZ594002, RZ594003, RZ594004, RZ594005, and RZ594006 with TOM modification at different sites of antisense strand, and the control conjugate RZ594001 without TOM modification were used in the present example to replace the siRNA conjugates used in Example 3; and that target gene FXI detection primers shown in TABLE 9 were used in the present example to replace target gene detection primers used in Example 3. Relative quantitative calculation of the gene mRNA of interest in various test groups was carried out according to the preceding ΔΔCt method.

TABLE 9
Primer Sequence Table

| Target Point | Primer Type | Primer Sequence (5′-3′) | Sequence ID |
|---|---|---|---|
| FXI | Upstream Primer | GCCCTGTTAAAACTGGAATCAGC | SEQ ID NO: 55 |
| | Downstream Primer | GCCCTGTTAAAACTGGAATCAGC | SEQ ID NO: 56 |
| GAPDH | Upstream Primer | TGCACCACCAACTGCTTAG | SEQ ID NO: 53 |
| | Downstream Primer | GGATGCAGGGATGATGTTC | SEQ ID NO: 54 |

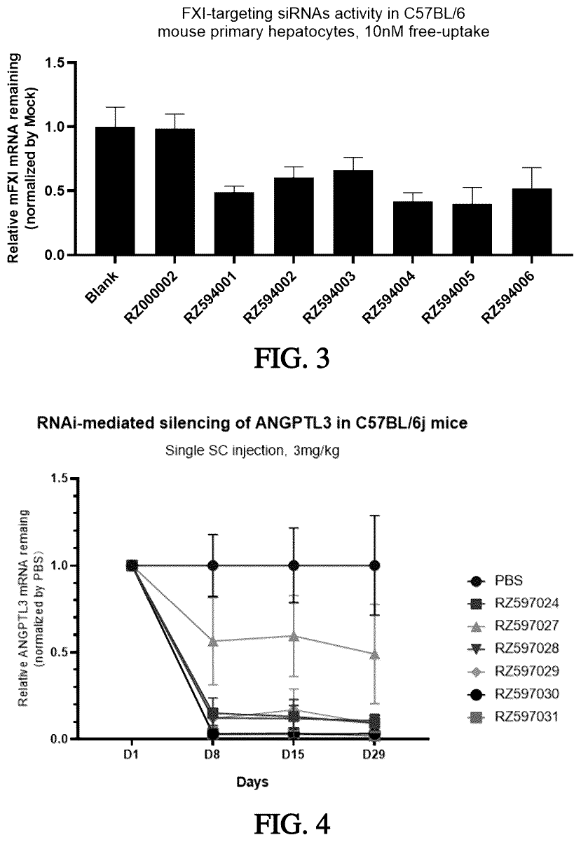

Results of Example 4 show that the TOM modifications at site 5 (RZ594002), site 6 (RZ594003), site 7 (RZ594004), site 8 (RZ594005), and site 9 (RZ594006) of the siRNA antisense strand have substantially comparable in vitro activity to the control sequence (RZ594006) without TOM modification, wherein RZ594004 and RZ594005 have better in vitro activity than the control sequence (RZ594001) without TOM modification (FIG. 3, TABLE 10).

TABLE 10
Inhibitory Activity of Mouse Primary Hepatocytes
on Target Gene FXI mRNA after Free Uptake of Various
Test Substances Tested in the Present Example

| Group | Mean Value of Inhibitory Rate | SD Value |
|---|---|---|
| Blank | 0.00% | ±0.1536 |
| RZ000002 | 1.53% | ±0.1145 |
| RZ594001 | 51.03% | ±0.0469 |
| RZ594002 | 39.72% | ±0.0858 |
| RZ594003 | 33.89% | ±0.0995 |
| RZ594004 | 57.94% | ±0.0651 |
| RZ594005 | 60.03% | ±0.1274 |
| RZ594006 | 47.90% | ±0.1594 |

### Example 5

Inhibitory Activity of siRNA Conjugates with TBDMS Modification at Different Sites of Antisense Strand on ANGPTL3 mRNA in Mice

The present example evaluated the inhibitory activity of siRNA conjugates RZ597027, RZ597028, RZ597029, RZ597030, and RZ597031 with TBDMS modification at different sites of antisense strand and control conjugate RZ597024 without TBDMS modification on the target gene ANGPTL3 of interest in mice by an evaluation method for inhibitory activity on a target gene in mice.

6-8 weeks old C57BL/6j mice were randomly grouped by body weight, 15 in each group and 7 groups in total. Mice in each group were administered with the above siRNA conjugates by subcutaneous administration into the abdomen, with a dosage of 3 mg/kg per mouse and a dose volume of 5 mL/kg. The same volume of PBS solution without siRNA conjugate was administered to PBS control group. The day of administration was recorded as first day (D1). After administration, five mice in each group were sacrificed at D8, D15 and D29, respectively. The animals were subjected to gross anatomy, and liver tissues were collected and cut into a plurality of 2 mm3 pieces and stored in RNAlater. An appropriate amount of liver tissue samples were taken out from RNAlater, smashed for 60 s in a Tissuelyser type II full-automatic tissue homogenizer, and subjected to RNA extraction, reverse transcription reaction and Real-time PCR detection by the detection method described in Example 3. Relative quantitative calculation was performed on the gene mRNA of interest in various test groups by the preceding ΔΔCt method.

Results of Example 5 show that the TBDMS modification at site 5 (RZ597027) of the siRNA antisense strand has a relatively large effect on the in vivo activity of the sequence, but the site 6 (RZ597028), site 7 (RZ597029), site 8 (RZ597030), and site 9 (RZ597031) of the antisense strand have good tolerance to TBDMS modification, RZ597028 and RZ597029 have comparable in vivo activity and duration to the control sequence (RZ597024) without TBDMS modification, and RZ597030 and RZ597031 have better in vivo activity and sustained pharmacodynamic effect than the control sequence (RZ597024) without TBDMS modification (FIG. 4, TABLE 11).

TABLE 11
Inhibitory Activity after Administration of the siRNA Conjugates
in the Present Example on Target Gene of Interest in Mice

| | D 8 | | D 15 | | D 29 | |
|---|---|---|---|---|---|---|
| | Mean Value of | | Mean Value of | | Mean Value of | |
| Group | Inhibitory Rate | SD Value | Inhibitory Rate | SD Value | Inhibitory Rate | SD Value |
| PBS | 0.00% | ±0.1782 | 0.00% | ±0.2155 | 0.00% | ±0.2871 |
| RZ597024 | 84.93% | ±0.087 | 86.98% | ±0.0642 | 90.75% | ±0.0444 |
| RZ597027 | 43.57% | ±0.2508 | 40.57% | ±0.2338 | 51.02% | ±0.2852 |
| RZ597028 | 87.83% | ±0.0427 | 88.08% | ±0.1092 | 89.25% | ±0.0374 |
| RZ597029 | 88.08% | ±0.0232 | 83.05% | ±0.1206 | 90.96% | ±0.0489 |
| RZ597030 | 96.83% | ±0.0176 | 96.86% | ±0.0121 | 96.76% | ±0.0073 |
| RZ597031 | 96.96% | ±0.0032 | 96.66% | ±0.0026 | 97.99% | ±0.0061 |

### Example 6

Inhibitory Activity of siRNA Conjugates with TOM Modification at Different Sites of Antisense Strand on CC3 mRNA in Mice

The present example evaluated the inhibitory activity of siRNA conjugates RZ502014 and RZ502015 with TOM modification at different sites of antisense strand and control conjugate RZ002001 without TOM modification on the target gene CC3 of interest in mice by an evaluation method for inhibitory activity on a target gene in mice.

According to the in vivo experimental method described in Example 5, 6-8 weeks old C57BL/6j mice were randomly grouped by body weight, 5 mice per group and 4 groups in total. The above siRNA conjugates were administered to each group of mice by subcutaneous administration into the abdomen, with a dosage of 3 mg/kg per mouse and a dose volume of 5 mL/kg. The same volume of PBS solution without siRNA conjugate was administered to PBS control group. The day of administration was recorded as first day (D1). After administration, the mice were sacrificed at D8. Liver tissues were collected to be subjected to RNA extraction, reverse transcription reaction and Real-time PCR detection. Relative quantitative calculation was performed on the gene mRNA of interest in various test groups by the preceding ΔΔCt method.

Differences lie in that the siRNA conjugates RZ502014 and RZ502015 with TOM modification at different sites of antisense strand, and control conjugate RZ002001 without TOM modification were used in the present example to replace the siRNA conjugate used in Example 5, and that the primer (CC3 target point) described in Example 1 was used to perform the Real-time PCR detection.

Results of Example 6 show that the site 7 (RZ502014) and site 9 (RZ502015) of the siRNA antisense strand have good tolerance to TOM modification, and the inhibitory activity on the target gene CC3 is only slightly weaker than that of the control sequence (RZ002001) without TOM modification (FIG. 5, TABLE 12).

TABLE 12
Inhibitory Activity after Administration of the siRNA Conjugates
in the Present Example on Target Gene of Interest in Mice

| Group | Mean Value of Inhibitory Rate | SD Value |
|---|---|---|
| PBS | 0.00% | ±0.2104 |
| RZ000002 | 77.47% | ±0.0817 |
| RZ502014 | 65.89% | ±0.1370 |
| RZ502015 | 57.31% | ±0.1127 |

### Example 7

Evaluation of On-Target Activity of siRNAs with TBDMS Modification at Different Sites of Antisense Strand in In Vitro psiCHECK System

According to an in vitro siRNA off-target evaluation method described by Ui-Tei, K. et al. (Functional dissection of siRNA sequence by systematic DNA substitution: modified siRNA with a DNA seed arm is a powerful tool for mammalian gene silencing with significantly reduced off-target effect. Nucleic Acids Res, 2008. 36 (7): p. 2136-51), psiCHECK plasmid for evaluation of siRNA on-target effect was constructed, and inhibitory activity of the tested siRNAs on target sequence was reflected by a dual-luciferase reporter system.

The present example evaluated on-target activity of siRNA conjugates RX597002, RX597003, RX597004, RX597005, RX597006, RX597007, and RX597008 with TBDMS modification at different sites of antisense strand, and control conjugate RX597001 without TBDMS modification on a target sequence in the psiCHECK system.

### 7.1 Plasmid Construction:

A corresponding target sequence was designed according to a base sequence of RX597001, and this target sequence was completely reversely complementary to RX597001 and antisense strands of various TBDMS modified siRNAs described in the present example; therefore, inhibitory effects of various siRNAs on the target sequence can reflect inhibitory actions of various siRNAs on target gene expression. Base composition of this target sequence was 5′-AGCCAAGAGCACCAAGAACTA-3′ (SEQ ID NO: 73), and this sequence was inserted into multiple cloning site of psiCHECK™-2 (Promega) to construct GSCM on-target plasmid. Construction and purification of this plasmid was entrusted to be completed by Beijing Tsingke Biotech Co., Ltd.

### 7.2 Formulation of Tested Articles:

(1) Formulation of siRNA tested articles: each of the above siRNA tested article was centrifuge and then dissolved by adding an appropriate amount of 1×PBS according to specification of each vial, and each siRNA tested article was further diluted with PBS to a total of 11 different working solution concentrations, i.e., 4000 nM, 1000 nM, 250 nM, 62.5 nM, 15.625 nM, 3.91 nM, 0.977 nM, 0.244 nM, 0.061 nM, 0.0153 nM and 0.0038 nM respectively.
(2) Formulation of plasmid: GSCM plasmid was diluted with PBS into a working solution concentration of 200 ng/μL.

### 7.3 Cell Culturing and Plating:

HEK293A cells were cultured and proliferated in DMEM culture medium containing 10% FBS in a 37° C., 5% CO₂ incubator. Before plating, the culture medium was discarded, and rinsing was performed with 0.25% pancreatin. After the cells were digested by pancreatin, digestion of the culture medium was terminated. The cells were resuspended, centrifuged at 800 rpm for 5 min, resuspended with fresh DMEM culture medium containing 10% FBS, counted, adjusted to 8×10⁴ cells/mL, plated into a 96-well plate, with 100 μL per well and 8×10³ cells per well, and further cultured for 24 h and then subjected to a transfection operation. The DMEM culture medium in the culture plate was discarded before transfection and replaced with 80 μL of Opti-MEM culture medium.

### 7.4 Cell Transfection:

7.4.1 Formulation of solution 1-mixed solution of siRNA and plasmid: for 11 working solutions of each siRNA tested article at different concentrations, 1 μL of the siRNA working solution at each concentration and 0.05 μL of plasmid working solution (200 ng/μL) were taken for each cell replicate well, added into 8.95 μL of Opti-MEM culture medium, mixed well, and formulated into solution 1 for transfecting one cell well. Each siRNA tested article at each concentration was prepared with 3 cell replicate wells of solution 1.

7.4.2 Formulation of solution 2-Lipofectamine 3000 mixed solution: gently mixing transfection reagent upside down, mixing according to 9.8 μL of Opti-MEM and 2 μL of Lipo3000 per cell well, gently pipetting 3-5 times to mix uniformly, and standing for 5 min. Each siRNA tested article at each concentration was prepared with 3 cell replicate wells of solution 2.

7.4.3 Formulation of solution 3-mixture of solution 1 and solution 2: gently mixing one part (10 μL) of the solution 1 with one part (10 μL) of the solution 2 to formulate solution 3, (20 μL) transfection complex, and incubating at room temperature for 20 min.

A cell culture plate to be transfected was taken out, original culture medium was discarded, and 80 μL of Opti-MEM was added. The solution 3 (20 μL) having undergone the incubation was added dropwise into cell culture wells to be formulated into a transfection system with each siRNA tested article at concentrations of about 40 nM, 10 nM, 2.5 nM, 0.625 nM, 0.15625 nM, 0.0391 nM, 0.00977 nM, 0.00244 nM, 0.00061 nM, 0.000153 nM and 0.00038 nM, respectively. The cell culture plate was further cultured in the 37° C., 5% CO₂ incubator for 4 h, and 1 mL of DMEM culture medium containing 20% FBS was supplemented to each well. Further culturing was carried out in the 37° C., 5% CO₂ incubator for 24 h.

Transfection complex group containing only plasmid but without siRNA was taken as a blank control group.

7.5 Detection:

The culture medium in the 96-well culture plate was discarded, LAR II substrate was added according to a method described in kit instructions of Dual-Glo® Luciferase Assay System (Promega), and incubation was performed on a shaker at room temperature for 10 min; 120 μL of substrate I was transferred onto a 96-well ELISA plate, and a Firefly chemiluminescence value was read on Synergy H1 microplate reader (Biotek); 60 μL of Stop&Glo substrate was further added into each well, incubation was performed on a shaker at room temperature for 10 min, and Renilla chemiluminescence value was read on the microplate reader. The inhibitory activity of the siRNA was calculated according to the Firefly and Renilla luminescence values detected in each well, and a calculation method is as follows:

Luminescence relative value per well: Ratio=Renilla/Firefly

$$Target relative expression level of siRNA tested article group = ( Ratio ( tested article ) / Ratio ( control ) ) \times 100 \%$$

$$Inhibitory rate of siRNA of sequence of interest = ( 1 - target relative expression level of siRNA tested article group ) \times 100 \% .$$

Calculation of half maximal inhibitory concentration (IC50): according to the target relative expression level of each siRNA tested article at different concentrations, a log (inhibitor) vs. response-Variable slope (four parameters) dose-effect curve was fitted using a Nonlinear regression (curve fit) analysis function of GraphPad Prism 8.0, to render IC50 calculation formula Y=Bottom+ (Top−Bottom)/(1+10\hat{ }((LogIC50−X)*HillSlope)).

In the above, Y is the target relative expression level, with a value of 50; X is a log logarithmic value of transfected siRNA concentration; Bottom is a Y value at bottom of a steady-state phase; Top is a Y value at top of the steady-state phase, LogIC50 is X value corresponding to the Y value halfway between the bottom and top of the steady-state phase, and HillSlope is slope of the curve at LogIC50.

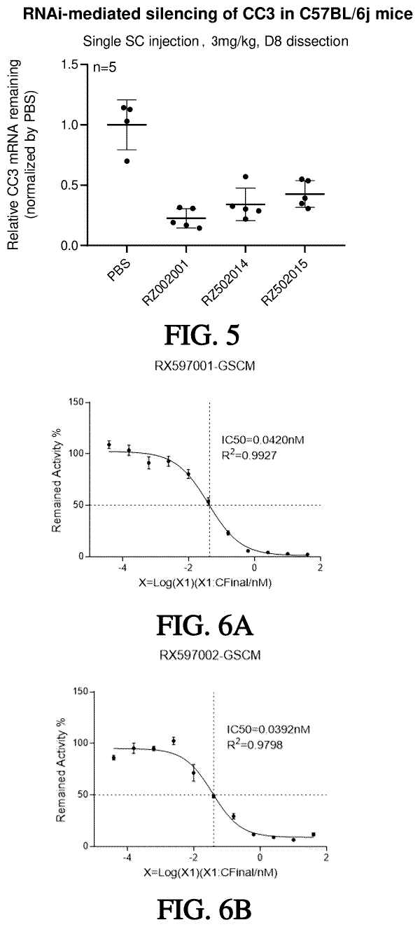

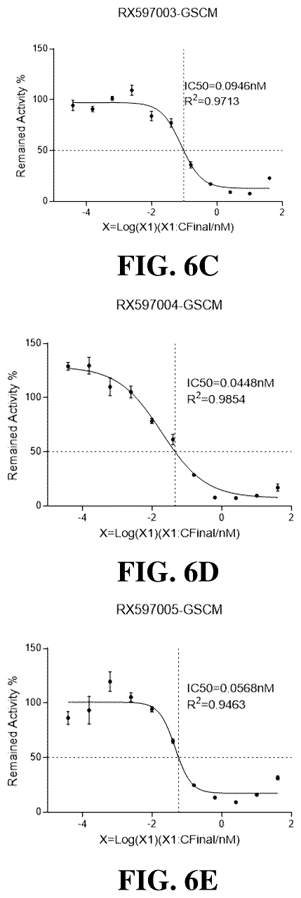

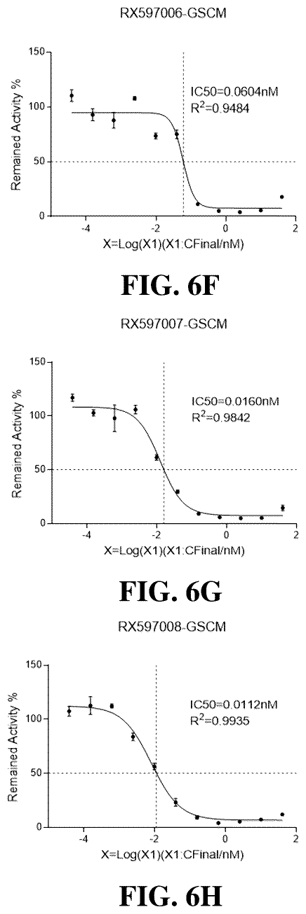

Results of Example 7 show that the site 3 (RX597002), site 4 (RX597003), site 5 (RX597004), site 6 (RX597005), site 7 (RX597006), site 8 (RX597007), and site 9 (RX597008) of the siRNA antisense strand all have good tolerance to TBDMS modification, and have substantially comparable in vitro psiCHECK activity to the control sequence (RX597001) without TBDMS modification, wherein psiCHECK system of RX597007 (IC50=0.0160) and RX597008 (IC50=0.0112) have obviously superior in vitro activity to RX597001 (IC50=0.0420) (FIGS. 6A-6H, TABLE 13).

TABLE 13
On-target Activity IC50 Values Detected by In vitro
psiCHECK System after Administration of Various
siRNA Tested Articles Tested in the Present Example

| Group | IC50 (nM) | R² |
|---|---|---|
| RX597001 | 0.0420 | 0.9927 |
| RX597002 | 0.0392 | 0.9798 |
| RX597003 | 0.0946 | 0.9713 |
| RX597004 | 0.0448 | 0.9854 |
| RX597005 | 0.0568 | 0.9463 |
| RX597006 | 0.0604 | 0.9484 |
| RX597007 | 0.0160 | 0.9842 |
| RX597008 | 0.0112 | 0.9935 |

### Example 8

Off-Target Activity of siRNAs with TBDMS Modification at Different Sites of Antisense Strand in In Vitro psiCHECK System

Off-target activity of siRNA conjugates RX597002, RX597003, RX597004, RX597005, RX597006, RX597007, and RX597008 with TBDMS modification at different sites of antisense strand and control conjugate RX597001 without TBDMS modification in psiCHECK system was evaluated. The present example merely differs from Example 7 in that the off-target evaluation plasmid (GSSM plasmid) used in the present example was inserted into a sequence 5′-CTAACCTCTACACAAGAACTA-3′ (SEQ ID NO: 74), which sequence was only reversely complementarily paired with nucleotides at sites 1-9 of the siRNA antisense strand from a 5′-end, and all of remaining sites were base mismatches; therefore, it can be used for miRNA-like off-target effect simulation evaluation of siRNA. Higher inhibitory activity of the siRNA test substance on the inserted sequence of interest indicates stronger miRNA-like off-target effect.

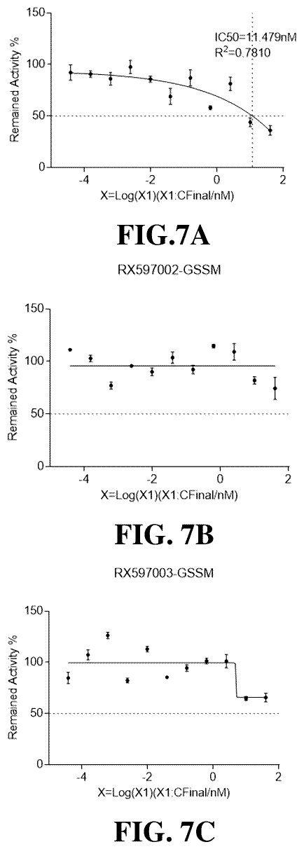

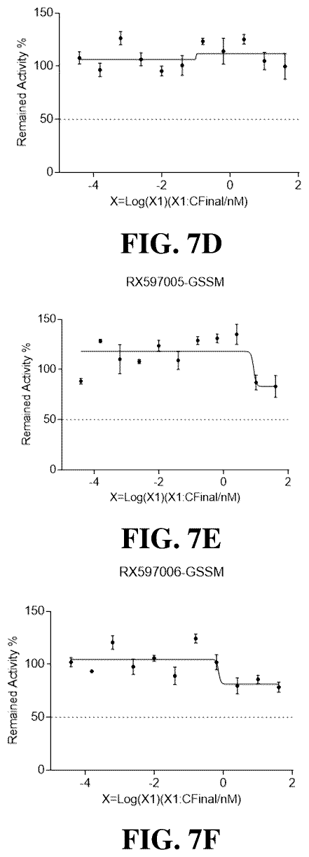

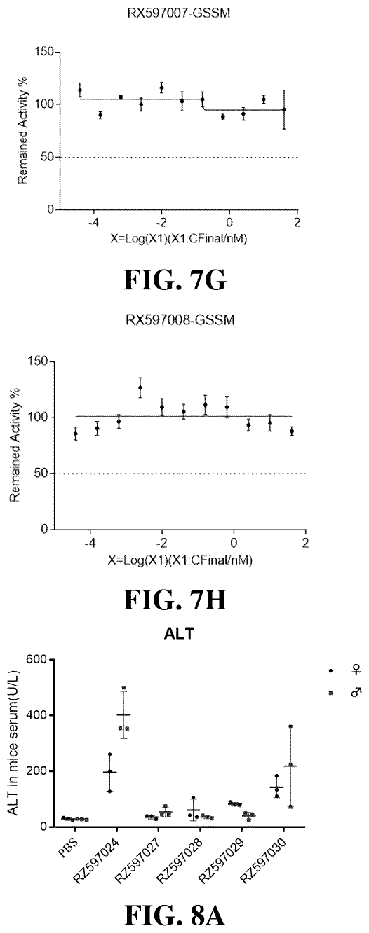

Results of Example 8 show that the control sequence (RX597001) without TBDMS modification has miRNA-like off-target effect, with an off-target IC50 value of 11.479 Nm. While the off-target IC50 value is not detected in the siRNA test substances modified with TBDMS at site 3 (RX597002), site 4 (RX597003), site 5 (RX597004), site 6 (RX597005), site 7 (RX597006), site 8 (RX597007), and site 9 (RX597008) of the antisense strand, indicating that this modification scheme significantly improves or eliminates the miRNA-like off-target effect of the control sequence while maintaining or even improving activity of the sequence (FIGS. 7A-7H, TABLE 14).

TABLE 14
Off-target Activity IC50 Values Detected by In vitro
psiCHECK System after Administration of Various siRNA
Test Substances Tested in the Present Example

| Group | IC50 (nM) | R2 |
|---|---|---|
| RX597001 | 11.479 | 0.7810 |
| RX597002 | Undetected | N/A |
| RX597003 | Undetected | N/A |
| RX597004 | Undetected | N/A |
| RX597005 | Undetected | N/A |
| RX597006 | Undetected | N/A |
| RX597007 | Undetected | N/A |
| RX597008 | Undetected | N/A |

### Example 9

Evaluation of Toxicity of siRNAs with TBDMS Modification at Different Sites of Antisense Strand in Mice:

The present example evaluated hepatotoxic effects of siRNA conjugates RZ597027, RZ597028, RZ597029, and RZ597030 with TBDMS modification at different sites of siRNA antisense strand and control conjugate RZ597024 without TBDMS modification in ICR mice.

6-8 weeks old ICR mice were randomly group according to body weight, 6 mice in each group and half females and half males. Mice in each group were administered with the above siRNA conjugates by subcutaneous injection into back of neck, with a dosage of 300 mg/kg and a dose volume of 10 mL/kg. PBS control group was administered with the same volume of PBS solution without siRNA conjugate. The day of administration was recorded as (first day) D1, and orbital blood of each mouse was sampled in an amount of 0.6 mL on D8 after the administration. Sampled blood was incubated at 37° C. for 60 min, and then centrifuged at 3000 rpm at 4° C. for 15 min to render serum. Serum samples were sent to Beijing Sinogenetic Biotechnology Co., Ltd. to detect concentrations of liver function indexes alanine aminotransferase (ALT) and aspartate aminotransferase (AST).

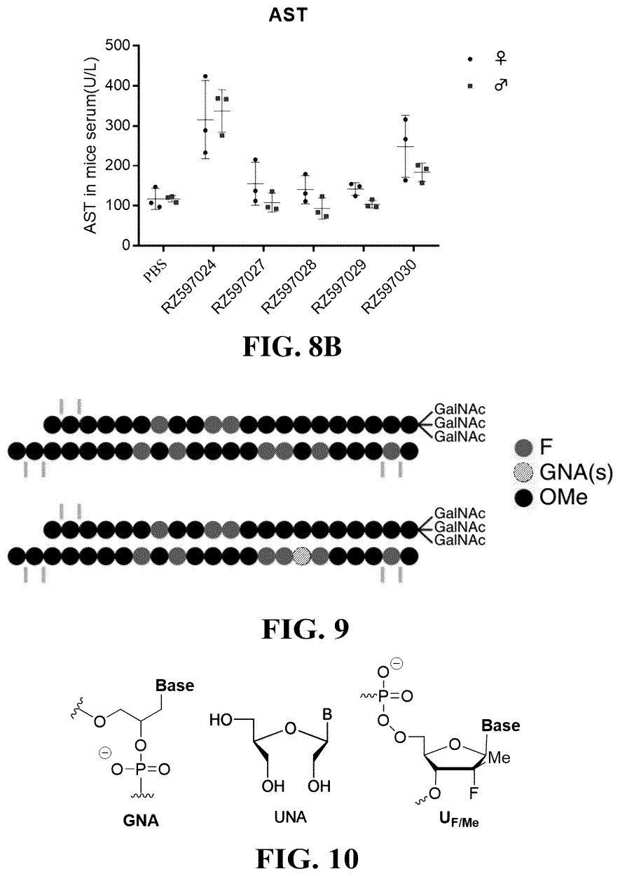

Results of Example 9 show that, compared with the PBS control group, after administration of 300 mg/kg siRNA conjugate (RZ597024) without TBDMS modification to the ICR mice, significant increases in levels of ALT and AST appeared in both female and male mice, indicating that the siRNA conjugate has relatively severe hepatotoxicity. However, compared with the group RZ597024, the siRNA conjugates with TBDMS modification at site 5 (RZ597027), site 6 (RZ597028), and site 7 (RZ597029) have significantly reduced levels of ALT and AST, close to that of the PBS control group, indicating that the modified compounds significantly improve the hepatotoxicity. RZ597030 (TBDMS modification at site 8 of antisense strand) also has a tendency to improve levels of ALT and AST (FIG. 8A and FIG. 8B).

### Example 10

Determination of Double-Stranded Melting temperatureS (Tm valueS) of siRNAs with TBDMS Modification at Different Sites of Antisense Strand:

The present example evaluated double-strand melting temperatureS (Tm valueS) of siRNA conjugates RZ597027, RZ597028, and RZ597029 with TBDMS modification at different sites of the siRNA antisense strand, and control conjugate RZ597024 without TBDMS modification. By formulating the above siRNA conjugates into 0.02 mg/mL aqueous solutions, temperature-absorbance curves at 260 nm wavelength were determined on a UV-Vis dual-cell temperature-controlled detector module of Agilent Cary UV workstation. Program set a spectral bandwidth of 2 nm, a starting temperature of 20° C., a heating rate of 0.5° C./min, and an ending temperature of 95° C. The double-strand thermal melting temperatures Tm were calculated from first-order derivative of the temperature-absorbance curves according to instrument instructions. Tm values of various siRNA conjugates, and ΔTm value of the control conjugate RZ597024 without tndms modification were calculated as shown in TABLE 15 below.

TABLE 15
Double-stranded Melting Temperatures (Tm values) of siRNAs with
TBDMS Modification at Different Sites of Antisense Strand

| siRNA Conjugate | Tm | ΔTm |
|---|---|---|
| RZ597024 | 73.1 | N/A |
| RZ597027 | 73.8 | 0.7 |
| RZ597028 | 73.3 | 0.2 |
| RZ597029 | 73.4 | 0.3 |

Results of Example 10 show that, compared with the control conjugate RZ597024 without TBDMS modification, the siRNA conjugates with TBDMS modification at site 5 (RZ597027), site 6 (RZ597028), and site 7 (RZ597029) of antisense strand have slightly increased Tm value, indicating that thermodynamic stability of the siRNA conjugates after the above TBDMS modification remains stable or is slightly improved.

### Example 11

Determination of Double-Stranded Melting Temperature (Tm Value) of siRNAs with TOM Modification at Different Sites of Antisense Strand:

By the same experimental method as that in Example 10, the present example evaluated the double-strand melting temperatures (Tm values) of siRNA conjugates RZ502014 and RZ502015 with TOM modification at different sites of siRNA antisense strand, and control conjugate RZ002001 without TOM modification. A difference merely lies in that, the siRNA conjugates RZ502014, RZ502015, and RZ002001 were used in the present example to replace the siRNA conjugates in Example 10.

Tm values of various siRNA conjugates, and ΔTm value of the control conjugate RZ002001 without TOM modification in the present example were calculated as shown in TABLE 16 below.

TABLE 16
Double-stranded Melting Temperatures (Tm values) of siRNAs with
TOM Modification at Different Sites of Antisense Strand

| siRNA Conjugate | Tm | ΔTm |
|---|---|---|
| RZ002001 | 85.5 | N/A |
| RZ502014 | 86.5 | 1 |
| RZ502015 | 85.5 | 0.0 |

Results of Example 11 show that, compared with the control conjugate RZ002001 without TOM modification, the siRNA conjugates with TOM modification at site 7 (RZ502014) AND site 9 (RZ502015) of antisense strand have Tm values substantially remaining stable or slightly increased, indicating that the above TOM modification method substantially does not affect thermodynamic stability of the double-stranded siRNA conjugates.

### Preparation of Compounds

Unless otherwise noted, reagents used in the preparation of compounds of the present disclosure were all purchased from Beijing Ouhe Technology Co., Ltd, where information of the main reagents is as shown in TABLE 17.

TABLE 17
Main Reagents

| Reagent Name | Abbreviation | CAS Number |
|---|---|---|
| Lithium aluminum hydride (LiAlH₄) | — | 16853-85-3 |
| Wet palladium on carbon (load amount 10 mass %) | Pd/C | — |
| Palladium hydroxide on carbon (load amount 10 mass %) | Pd(OH)₂/C | — |
| Benzotriazole-N,N,N',N'-tetramethyluronium | HBTU | 94790-37-1 |
| hexafluorophosphate | | |
| 2-(7-azobenzotriazole-N,N,N',N'-tetramethyluronium | HATU | 148893-10-1 |
| hexafluorophosphate | | |
| 4,4'-bismethoxy trityl chloride/4,4'-dimethoxy | DMTrCl | 40615-36-9 |
| triphenylchloromethane | | |
| Bis(diisopropylamino)(2-cyanoethoxy)phosphine | — | 102691-36-1 |
| 4,5-dicyanoimidazole | DCI | 1122-28-7 |
| 4-dimethylaminopyridine | DMAP | 1122-58-3 |
| Amino CPG (Aminoalkyl-CPG, model C3006-1000) | 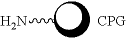 | — |
| 1,4-dioxane solution in 4 M hydrochloric acid | — | — |
| Trans-4-(Boc-amino)cyclohexanecarboxaldehyde | — | 181308-57-6 |
| N-carbobenzoxy-4-aminobutyric acid | — | 5105-78-2 |
| 5-[[(2R,3R,4R,5R,6R)-3-acetamido-4,5-diacetoxy-6- | Compound 4 | 1159408-54-4 |
| (acetoxymethyl)-2-tetrahydropyranyl]oxy]pentanoic acid | | |

In the above, CPG represents controlled pore glass carrier.

### Preparation Example 1 Preparation of Compound CR01008 and Compound CR01008Z

### (1.1) Synthesis of Compound CR01008

In the present preparation example, a synthesis route of the compound CRO1008 was as follows:

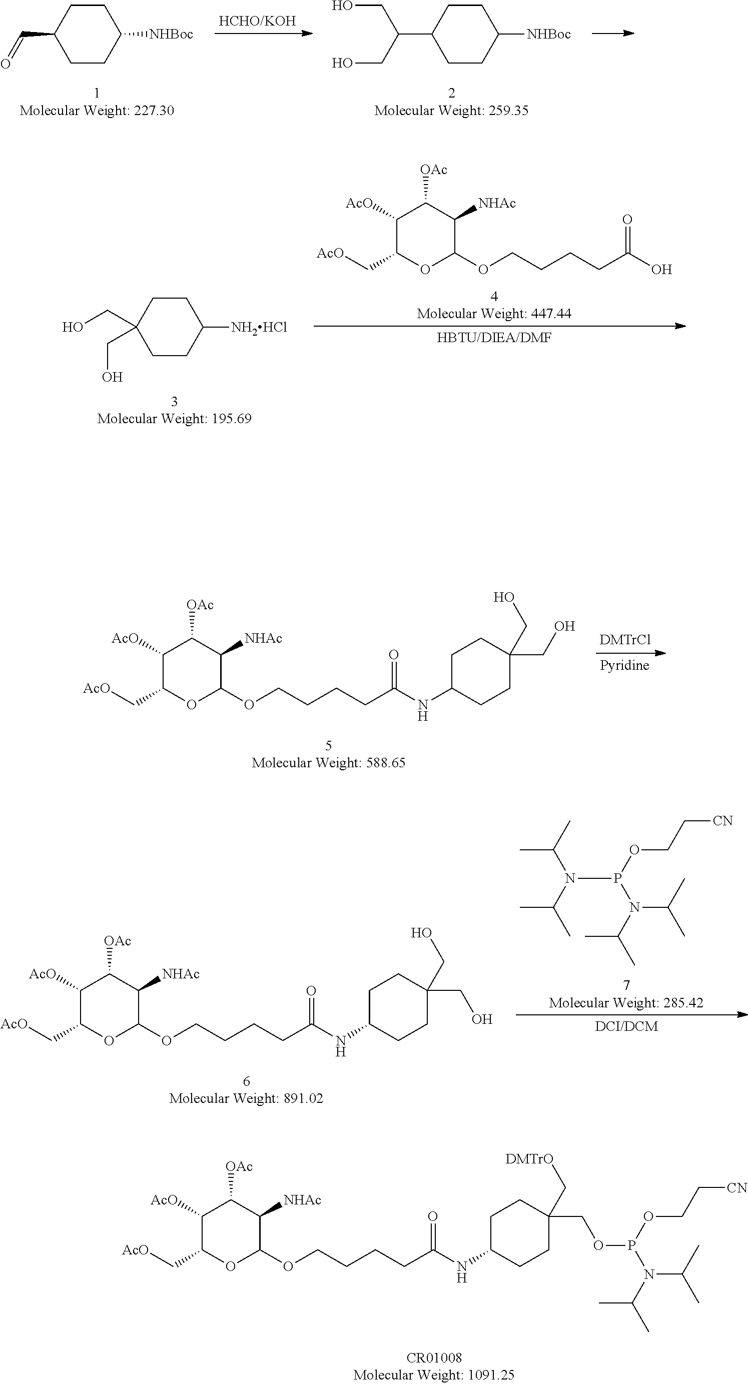

### (1.1.1) Synthesis of Compound 2

The compound 1 (trans-4-(Boc-amino)cyclohexanecarboxaldehyde, 10.0 g, 1.0 eq) and formaldehyde aqueous solution (8.9 g, 37 mass %, 2.4 eq) were dissolved in 33 ml of methanol. 13 ml of 45.3 mass % KOH aqueous solution was added dropwise. After the dropwise addition was completed, mixture was stirred and reacted at 25° C. for 30 min, heated to 60° C. and subjected to reflux reaction at 60° C. for 2 h. After the reaction was ended, a reaction liquid was cooled to room temperature and then evaporated to dryness under reduced pressure to render a white solid-like crude product. The crude product was slurried by adding a small amount of water and filtered to render white solid-like compound 2 (9 g, yield 78.9%). MS-ESI (m/z)=260 [M+H]⁺.

### (1.1.2) Synthesis of Compound 3

The compound 2 (9 g, 1 eq) prepared according to step (1.1.1) was dissolved in 70 ml of 1,4-dioxane, 1,4-dioxane solution (45 ml, 4 M) of hydrogen chloride was added, and mixture was stirred and reacted at 25° C. for 1 h. After the reaction was ended, a reaction liquid was evaporated to dryness under reduced pressure to render white solid-like compound 3 (6.8 g, yield 100%).

### (1.1.3) Synthesis of Compound 5

The compound 3 (1.8 g, 2.0 eq) prepared according to step (1.1.2), compound 4 (5-[[(2R,3R,4R,5R,6R)-3-acetamido-4,5-diacetoxy-6-(acetoxymethyl)-2-tetrahydropyranyl]oxy]pentanoic acid, 2.1 g, 1.0 eq) and DIEA (N,N-diisopropylethylamine, 3.5 g, 6.0 cq) were dissolved in 15 ml of DMF, HBTU (1.9 g, 1.1 eq) was added, and mixture was stirred and reacted at 25° C. for 3 h in a N2 atmosphere. After the reaction was ended, a reaction liquid was evaporated to dryness under reduced pressure and reversely purified (22 volume % acetonitrile aqueous solution) to render white solid-like compound 5 (1.78 g, yield 64.4%). MS-ESI (m/z)=589 [M+H]⁺.

### (1.1.4) Synthesis of Compound 6

The compound 5 (1.54 g, 1.0 eq) prepared according to step (1.1.3) was dissolved in 15 ml of pyridine, reaction system was cooled to 0° C. using ice water bath and DMTrCl (4,4′-dimethoxy triphenylchloromethane, 1.32 g, 1.5 eq) was added at 0° C. Reaction was carried out at 25° C. for 3 h, and 15 ml of methanol was added to reaction liquid to quench the reaction. After the reaction was ended, a reaction liquid was evaporated to dryness under reduced pressure and reversely purified (aqueous solution of 60 volume % acetonitrile) to render yellow solid-like compound 6 (1 g, yield 42.7%). MS-ESI (m/z)=891 [M+H]⁺.

### (1.1.5) Synthesis of Compound CR01008

The compound 6 (1.08 g, 1.0 eq) prepared according to step (1.1.4) was dissolved in 20 ml of anhydrous dichloromethane, DCI (115 mg, 0.8 eq) and compound 7 (bis(diisopropylamino) (2-cyanoethoxy)phosphine, 732 mg, 2.1 eq) were added respectively, nitrogen replacement was carried out three times, and resultant was stirred and reacted at 25° C. for 2 h. After the reaction was ended, 20 ml of saturated sodium bicarbonate aqueous solution was added to reaction liquid, extraction was performed with 20 ml of dichloromethane three times (3×20 ml), and organic phases were combined. The organic phases were evaporated to dryness under reduced pressure, reversely purified (aqueous solution of 72 vol % acetonitrile), and then vacuum-dried for 12 h to render white powdered compound CR01008 (1 g, yield 76.0%). MS-ESI (m/z)=1091 [M+Na]⁺.

1H NMR (400 MHZ, DMSO-d6) δ 1.05 (d, J=6.7 Hz, 6H), 1.14 (d, J=6.7 Hz, 6H), 1.37-1.17 (m, 5H), 1.60-1.40 (m, 6H), 1.68-1.62 (m, 1H), 1.80 (s, 3H), 1.80 (s, 3H), 1.92 (s, 3H), 2.02 (s, 5H), 2.13 (s, 3H), 2.71 (t, J=5.9 Hz, 2H), 2.79 (d, J=8.4 Hz, 1H), 2.87 (d, J=8.4 Hz, 1H), 3.36 (s, 1H), 3.58-3.39 (m, 3H), 3.69-3.60 (m, 2H), 3.75 (s, 7H), 3.90 (dt, J=11.2, 8.8 Hz, 1H), 4.05 (s, 3H), 4.51 (d, J=8.4 Hz, 1H), 4.99 (dd, J=11.3, 3.4 Hz, 1H), 5.24 (d, J=3.4 Hz, 1H), 5.78 (s, 1H), 6.93-6.87 (m, 4H), 7.35-7.21 (m, 7H), 7.44-7.37 (m, 2H), 7.66 (d, J=7.8 Hz, 1H), 7.84 (d, J=9.2 Hz, 1H).

### (1.2) Synthesis of Compound CR01008Z

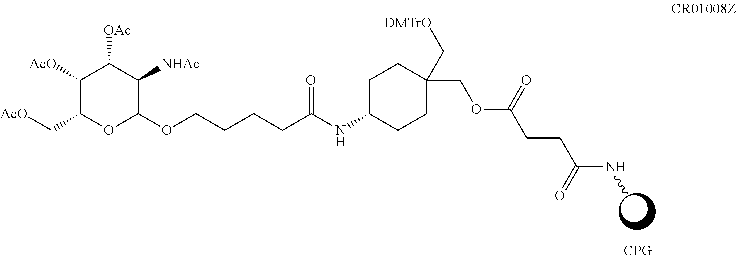

In the present preparation example, a synthesis route of the compound CR01008Z was as follows:

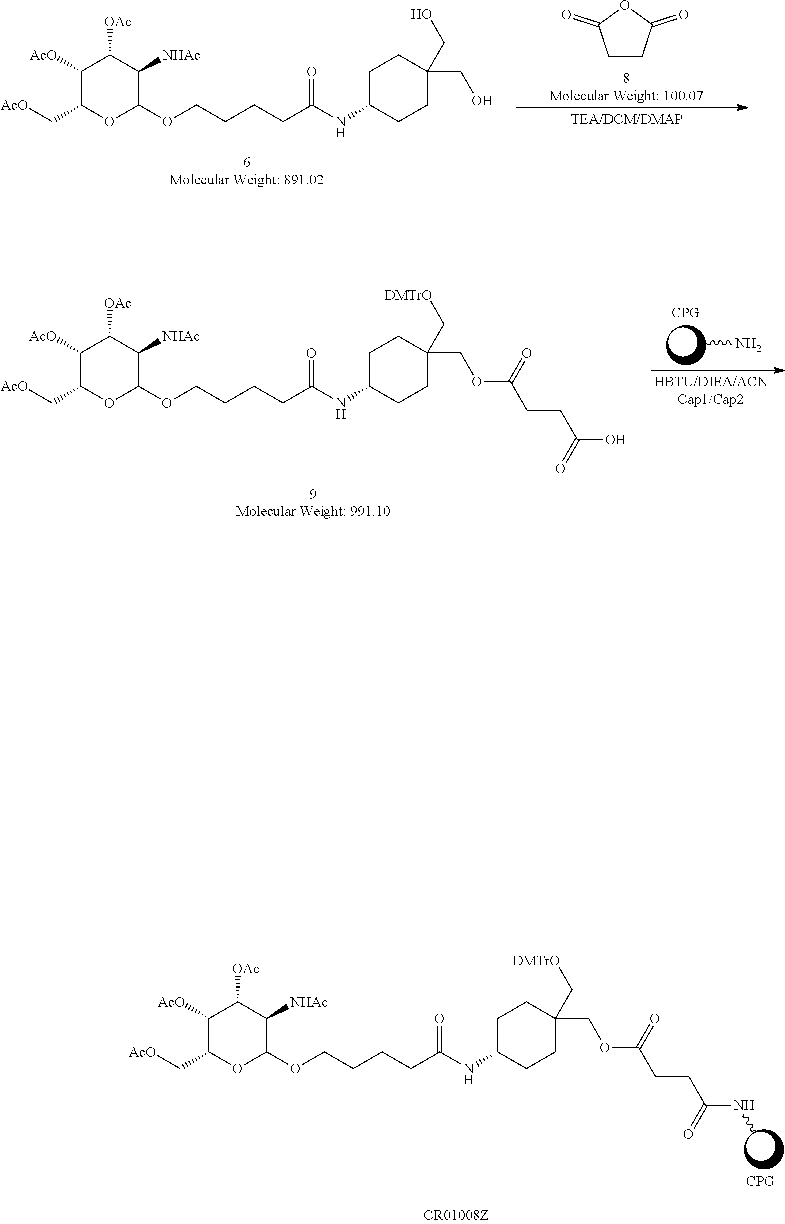

### (1.2.1) Synthesis of Compound 9

The compound 6 (500 mg) prepared according to step (1.1.4) was dissolved in 10 ml of dichloromethane, compound 8 (succinic anhydride, 112 mg), DMAP (6.8 mg) and TEA (226.2 mg) were added, nitrogen replacement was carried out three times. Resultant was stirred and reacted at 25° C. for 16 h, and subjected to flash-purification to render the compound 9 (300 mg, yield 53.6%). MS-ESI (m/z)=1013 [M+Na]⁺.

### (1.2.2) Synthesis of Compound CR01008Z

To a 20 ml sample vial, the compound 9 (50 mg, amino CPG (1.25 g, 80 μmol/g, 0.1 mmol) prepared according to step (1.2.1), HBTU (27 mg), and DIEA (12 mg) were added, and reacted on a shaker for 16 h. After the reaction was ended, a reaction liquid was filtered to render a filter cake. The filter cake was first washed once with 10 ml of acetonitrile (1×10 ml), and then dried in vacuum. To the 20 ml sample vial, the dried filter cake, DMAP (3 mg), Cap1 (10 ml, 200 V) and Cap2 (1 ml, 20 V) were added and reacted on a shaker for 6 h. After the reaction was ended, a reaction liquid was filtered to render a filter cake. The filter cake was first washed once with 10 ml of acetonitrile (1×10 ml), and then dried in vacuum to render the compound CR01008Z (1.03 g, load amount 20-30 μmol/g).

In the above, Cap1 and Cap2 were capping reagents, Cap1 was a pyridine/acetonitrile mixed solution of 20 volume % N-methylimidazole, a volume ratio of pyridine to acetonitrile being 3:5; and Cap2 was an acetonitrile solution of 20 volume % acetic anhydride.

### Preparation Example 2 Preparation of Compound CR01013 and Compound CR01013Z

### (2.1) Preparation of Compound CR01013

In the present preparation example, a synthesis route of the compound CR01013 was as follows:

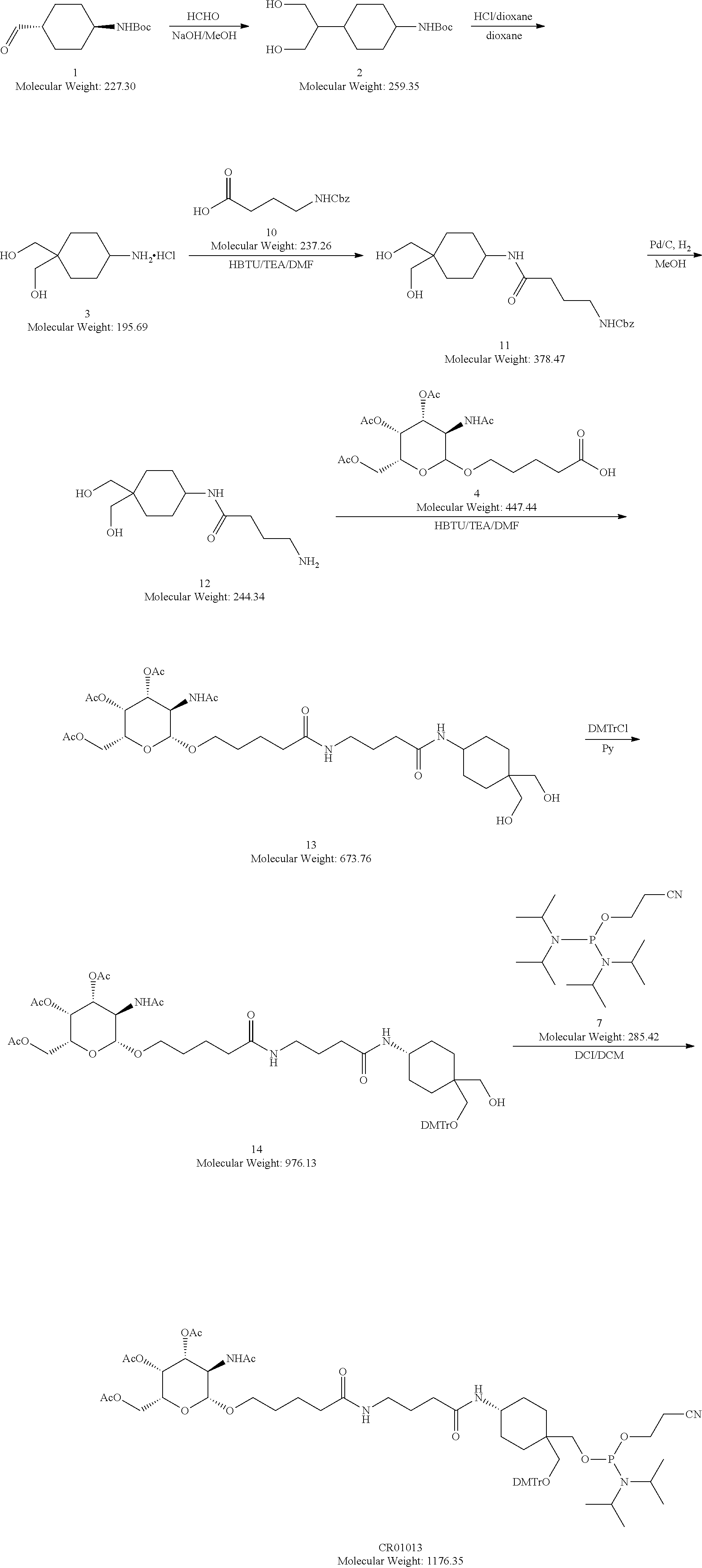

### (2.1.1) Synthesis of Compound 2

The compound 1 (trans-4-(Boc-amino)cyclohexanecarbaldehyde, 4.9 g) was dissolved in 17 ml of methanol, and an formaldehyde aqueous solution (4.21 g, concentration being 37 mass %) and a sodium hydroxide aqueous solution (6.5 ml, concentration being 45.3 mass %) were added dropwise. After the dropwise addition was completed, mixture was heated to 60° C. and stirred and reacted at 60° C. for 2 h. After the reaction was ended, a reaction liquid was cooled to 25° C. and then evaporated to dryness under reduced pressure to render a white solid-like crude product. The crude product was slurried by adding a small amount of water, filtered and then dried to render white solid-like compound 2 (4.8 g, yield 85.9%). ESI-MS (m/z)=260.2 [M+H]⁺.

### (2.1.2) Synthesis of Compound 3

The compound 2 (4.8 g) prepared according to step (2.1.1) was dissolved in 25 ml of 1,4-dioxane, 1,4-dioxane solution (25 ml, 4 M) of hydrochloric acid was added, and mixture was stirred and reacted at 25° C. for 2 h. After the reaction was ended, a reaction liquid was evaporated to dryness under reduced pressure to render white solid-like compound 3 (3.6 g, yield 99.4%).

### (2.1.3) Synthesis of Compound 11

The compound 3 (3.6 g) prepared according to step (2.1.2) was dissolved in 36 ml of DMF, TEA (5.62 g), compound 10 (N-carbobenzoxy-4-aminobutyric acid, 5.28 g), and HBTU (8.43 g) were added, and mixture was stirred and reacted at 25° C. for 16 h. After the reaction was ended, a reaction liquid was added into 200 ml of saturated sodium bicarbonate aqueous solution, extraction was performed with 100 ml of ethyl acetate three times (3×100 ml), and organic phases were combined. The organic phases were first washed once with 50 ml of saturated sodium chloride aqueous solution (1×50 ml), then dried with anhydrous sodium sulfate, evaporated to dryness under reduced pressure, and subjected to column chromatography normal phase purification (eluent: dichloromethane/methanol=10/1, v/v), to render white solid-like compound 11 (2.3 g, yield 33.0%). ESI MS (m/z)=379.5 [M+H]⁺.

### (2.1.4) Synthesis of Compound 12

The compound 11 (2.3 g) prepared in step (2.1.3) was dissolved in 23 ml of methanol, wet palladium on carbon (230 mg, load amount 10 mass %) was added, and hydrogen replacement was carried out three times. Reaction system was stirred and reacted at 25° C. for 16 h in a hydrogen atmosphere (15 psi). After the reaction was ended, a reaction liquid was filtered to render a filtrate, and the filtrate was evaporated to dryness under reduced pressure to render yellow oily compound 12 (1.48 g, yield 99.8%).

### (2.1.5) Synthesis of Compound 13

The compound 12 (1.48 g) prepared according to step (2.1.4) was dissolved in 15 ml DMF, triethylamine (TEA, 1.22 g), compound 4 (1.35 g), and HBTU (3.45 g) were added, and mixture was stirred and reacted at 25° C. for 16 h. After the reaction was ended, a reaction liquid was added into 150 ml of saturated sodium bicarbonate aqueous solution, extraction was performed with 50 ml of ethyl acetate three times (3×50 ml), and organic phases were combined. The organic phases were first washed once with 30 ml of saturated sodium chloride aqueous solution (1×30 ml), then dried with anhydrous sodium sulfate, evaporated to dryness under reduced pressure, and subjected to column chromatography reverse phase purification (C18 chromatographic column, eluent: water/acetonitrile=5/1, v/v), to render white solid-like compound 13 (1.3 g, yield 31.8%). ESI-MS (m/z): 674.3 [M+H]⁺.

### (2.1.6) Synthesis of Compound 14

The compound 13 (1.1 g) prepared according to step (2.1.5) was dissolved in 11 ml of pyridine, reaction system was cooled to 0° C. using an ice water bath, DMTrCl (813 mg) was added in batches at 0° C., and reaction system was stirred at 0° C. for 1 h. After the reaction was ended, methanol was added to reaction liquid to quench, and solvent was evaporated, and subjected to column chromatography reverse phase purification (eluent: water/acetonitrile=1/4, v/v) was carried out, to render white solid-like compound 14 (800 mg, yield 50.3%). ESI-MS (m/z): 976.5 [M+H]⁺.

### (2.1.7) Synthesis of Compound CR01013

At 25° C., the compound 14 (550 mg) prepared according to step (2.1.6) was dissolved in 5 ml of dichloromethane (DCM), 4,5-dicyanoimidazole (DCl, 53.2 mg) and compound 7 (2-cyanoethyl N,N,N′,N′-tetraisopropyl phosphoramidite, 255.4 mg) were added, nitrogen replacement was carried out three times, and reaction system was stirred at 25° C. for 1 h in a nitrogen atmosphere. After the reaction was ended, a reaction liquid was first washed with 5 ml of saturated sodium bicarbonate aqueous solution twice (2×5 ml), and then washed once with 30 ml of saturated sodium chloride aqueous solution (1×30 ml). Organic phases were separated, dried with anhydrous sodium sulfate, evaporated to dryness under reduced pressure, and subjected to column chromatography normal phase purification (eluent: dichloromethane/methanol=20/1, v/v), to render white solid-like compound CR01013 (532 mg, yield 80.4%). ESI-MS (m/z): 1176.7 [M+H]⁺.

1H NMR (400 MHZ, DMSO-d6) δ 0.95-1.05 (d, J=6.7 Hz, 5H), 1.06-1.15 (q, J=7.6 Hz, 8H), 1.15-1.21 (t, J=7.2 Hz, 14H), 1.72-1.80 (s, 3H), 1.84-1.92 (s, 3H), 1.94-2.07 (d, J=16.0 Hz, 7H), 2.07-2.14 (s, 3H), 2.64-2.72 (q, J=5.8 Hz, 2H), 2.74-2.89 (d, J=8.5 Hz, 2H), 3.35-3.56 (m, 4H), 3.57-3.70 (m, 4H), 3.71-3.77 (s, 6H), 3.81-3.93 (m, 1H), 3.96-4.09 (d, J=6.4 Hz, 3H), 6.82-6.97 (d, J=8.7 Hz, 4H), 7.17-7.27 (t, J=8.7 Hz, 5H), 7.27-7.34 (t, J=7.6 Hz, 2H), 7.34-7.43 (d, J=7.5 Hz, 2H).

### (2.2) Synthesis of Compound CR01013Z

In the present preparation example, a synthesis route of the compound CR01013Z was as follows:

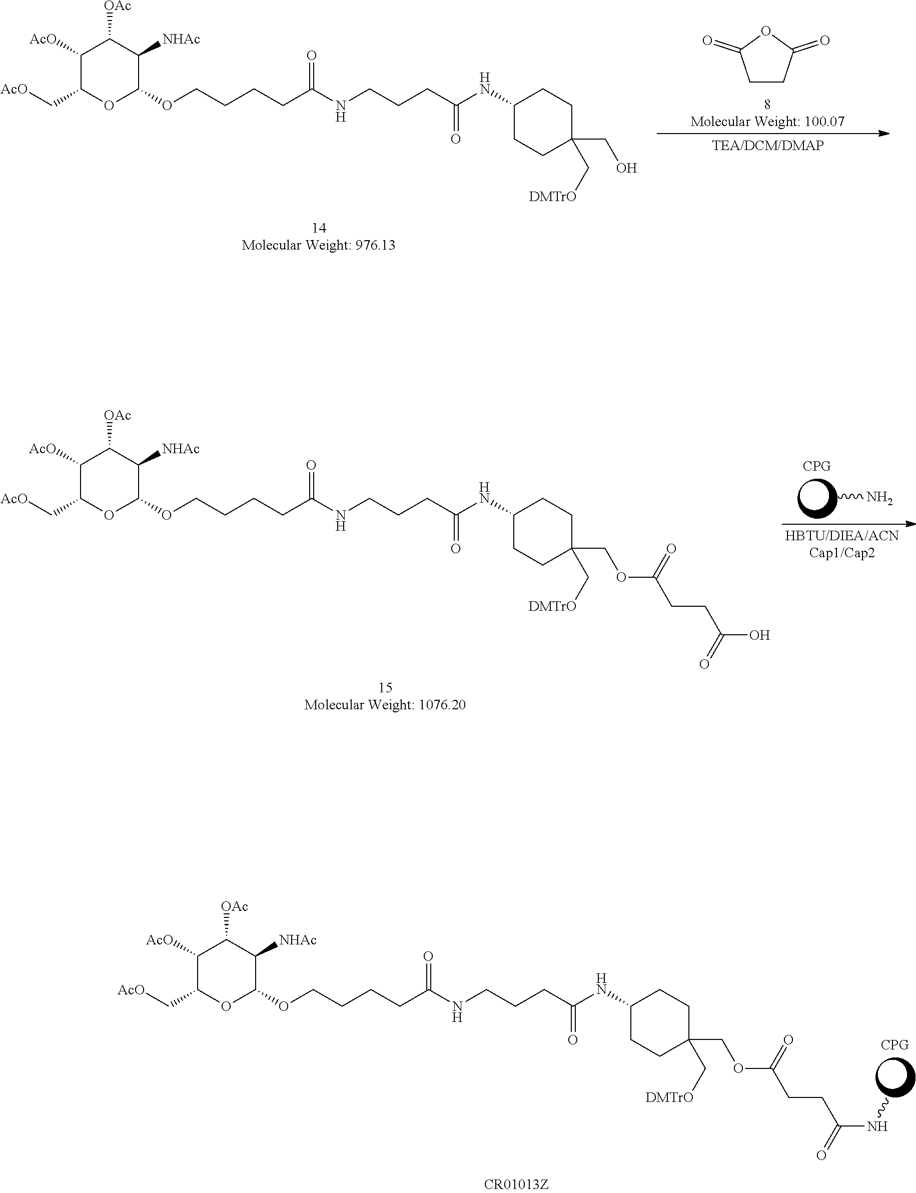

### (2.2.1) Synthesis of Compound 15

At 25° C., the compound 14 (100 mg, 0.10 mmol) prepared according to step (2.1.6) was dissolved in 2 ml of dichloromethane, triethylamine (25.9 mg, 0.25 mmol), DMAP (1.25 mg, 0.01 mmol), and compound 8 (succinic anhydride, 15.4 mg, 0.15 mmol) were added, and reaction system was stirred at 25° C. for 16 h. After the reaction was ended, solvent in a reaction liquid was evaporated, and column chromatography reverse phase purification (C18 chromatographic column, eluent: water/acetonitrile=2/1, v/v) was carried out, to render yellow oily compound 15 (110 mg, 0.10 mmol, yield 100%). ESI-MS (m/z)=1099.3 [M+Na]⁺.

### (2.2.2) Synthesis of Compound CR01013Z

The compound 15 (50 mg, 0.04 mmol) prepared according to step (2.2.1) was dissolved in 10 ml of acetonitrile, and HBTU (24.2 mg, 0.06 mmol), DIEA (11.0 mg, 0.08 mmol), and amino-CPG (1.06 g, load amount 80 μmol/g) were added, and reaction system was stirred and reacted at 25° C. for 16 h. After the reaction was ended, a reaction liquid was filtered to render a filter cake. The filter cake was first washed with 50 ml of dichloromethane twice (2×50 ml), 50 ml of acetonitrile three times (3×50 ml), and 50 ml of ethyl acetate once (1×50 ml) in sequence, and then dried in vacuum. To the dried filter cake, Cap1 (4.8 ml), Cap2 (0.54 ml) and DMAP (2.59 mg) were added. The reaction system was stirred and reacted at 25° C. for 5 h. After the reaction was ended, a reaction liquid was filtered to render a filter cake, and the filter cake was washed with 50 ml of acetonitrile three times (3×50 ml), and vacuum-dried to render the compound CR01013Z (900 mg, load amount 20-30 μmol/g).

In the above, Cap1 and Cap2 were capping reagents, Cap1 was a pyridine/acetonitrile mixed solution of 20 volume % N-methylimidazole, a volume ratio of pyridine to acetonitrile being 3:5; and Cap2 was an acetonitrile solution of 20 volume % acetic anhydride.

Preparation of siRNA Conjugates

Unless otherwise noted, synthesis of siRNA sequences used in the present disclosure was entrusted to be completed by Suzhou Biosyntech Co., Ltd.; and synthesis of PCR primers used in the present disclosure was entrusted to be completed by Beijing Tsinigke iotech Co., Ltd.

### Preparation Example 3: Preparation of siRNA Conjugates Conjugated with (CR01008×3) Carrier at 3′-End of Sense Strand

### (3.1) Synthesis of Sense Strand (SS)

By a method for solid phase synthesis of phosphoramidite nucleic acids, starting from the above compound CR01008Z or compound CR01013Z linked to a solid-phase carrier, nucleoside monomers were circularly linked one by one according to a nucleotide sequence in a direction of 3′-5′ (in a synthesis process, the compound CR01008 or the compound CR01013Z was regarded as a nucleoside monomer).

Each nucleoside monomer was linked through four-step reactions of deprotection, coupling, capping, oxidation or sulfurization. Synthesis conditions were given as follows.

The nucleoside monomer was formulated into an acetonitrile solution of nucleoside monomer at a concentration of 0.1 M.

Conditions of deprotection reaction in each step were the same. The conditions of the deprotection reaction were as follows: a temperature was 25° C., reaction time was 70 s, deprotection reagent was a dichloromethane solution (3 vol %) of dichloroacetic acid, and a molar ratio of dichloroacetic acid to 4,4′-dimethoxytrityl protecting group on a solid-phase carrier was 5:1.

Conditions of coupling reaction in each step were the same. The conditions of the coupling reaction were as follows: a temperature was 25° C., a molar ratio of nucleic acid sequences linked to a solid-phase carrier to nucleoside monomers was 1:10, a molar ratio of nucleic acid sequences linked to the solid-phase carrier to a coupling reagent was 1:65, reaction time was 600 s, a coupling reagent was an acetonitrile solution of 5-ethylthio-1H-tetrazole at a concentration of 0.5 M, and a thionating reagent was a acetonitrile/pyridine mixed solution of hydroxanthogen at a concentration of 0.2 M (a volume ratio of acetonitrile to pyridine was 1:1).

Conditions of capping reaction in each step were the same. The conditions of the capping reaction were as follows: a temperature was 25° C.; reaction time was 2 m; a capping reagent solution was a mixed solution of Cap1 and Cap2 in a molar ratio of 1:1, Cap1 was a pyridine/acetonitrile mixed solution of N-methylimidazole at a concentration of 20 volume %, a volume ratio of pyridine to acetonitrile was 3:5, and Cap2 was an acetonitrile solution of acetic anhydride at a concentration of 20 volume %; a molar ratio of N-methyl imidazole in Cap1 capping reagent, acetic anhydride in Cap2 capping reagent to nucleic acid sequences linked to a solid-phase carrier was 1:1:1.

Conditions of oxidation reaction in each step were the same. The conditions of the oxidation reaction were as follows: a temperature was 25° C.; reaction time was 3 s; an oxidation reagent was iodine solution at a concentration of 0.05 M, a molar ratio of iodine to nucleic acid sequences linked to a solid-phase carrier in the coupling reaction was 30:1; and the oxidation reaction was carried out in a water/pyridine mixed solvent (a volume ratio of water to pyridine was 1:9). Conditions of sulfurization were as follows: a temperature was 25° C.; reaction time was 360 s; a thionating reagent was pyridine solution of hydrogenated xanthogen at a concentration of 0.2 M, a molar ratio of the thionating reagent to nucleic acid sequences linked to a solid-phase carrier in the coupling reaction was 4:1; and a thionation reaction was carried out in a water/pyridine mixed solvent (a volume ratio of water to pyridine was 1:9).

After the last nucleoside monomer was linked, the nucleic acid sequences linked on the solid-phase carrier were cleaved, deprotected, purified, and desalted in sequence, and subsequently lyophilized to render a sense strand.

Conditions of cleavage and deprotection were as follows: the synthesized nucleotide sequences linked with the solid-phase carrier were added into ammonia water at a concentration of 25 mass %, an amount of the ammonia water being 0.5 ml/μmol, and reacted at 55° C. for 16 h, solvent was removed, and resultant was concentrated in vacuum to dryness. After treatment with the ammonia water, product was dissolved with 0.4 ml/μmol N-methylpyrrolidone relative to an amount of single-stranded nucleic acid.

Conditions of purification and desalting: purification of nucleic acid was completed by gradient elution of NaCl using a preparative ion chromatography purification column (Source 15Q). Specifically, eluent 1 was 20 mM sodium phosphate (pH=8.1), and solvent was a water/acetonitrile mixed solution (a volume ratio of water to acetonitrile was 9:1); eluent 2 was 1.5 M sodium chloride, 20 mM sodium phosphate (pH=8.1), and solvent was a water/acetonitrile mixed solution (a volume ratio of water to acetonitrile was 9:1); and elution gradient was eluent 1: eluent 2=(100:0)-(50:50). Product eluates were collected and combined, and desalted using a reverse chromatography purification column, where desalting conditions included desalting using a dextran gel column, a filler being dextran gel G25, and eluting with deionized water.

Detection: purity was detected using ion exchange chromatography (IEX-HPLC); and molecular weight was detected using liquid chromatography-mass spectrometry (LC-MS). An actually measured value and a theoretical value of the molecular weight were compared, and if the actually measured value and the theoretical value were consistent, it indicated that the sense strand of the siRNA was obtained.

Trimer of CR01008, denoted as (CR01008)×3 or (CR01008×3) or (CR01008) (CR01008) (CR01008), were synthesized in the synthesis of the present step.

Structural formula of the trimer of CR01008 is as follows:

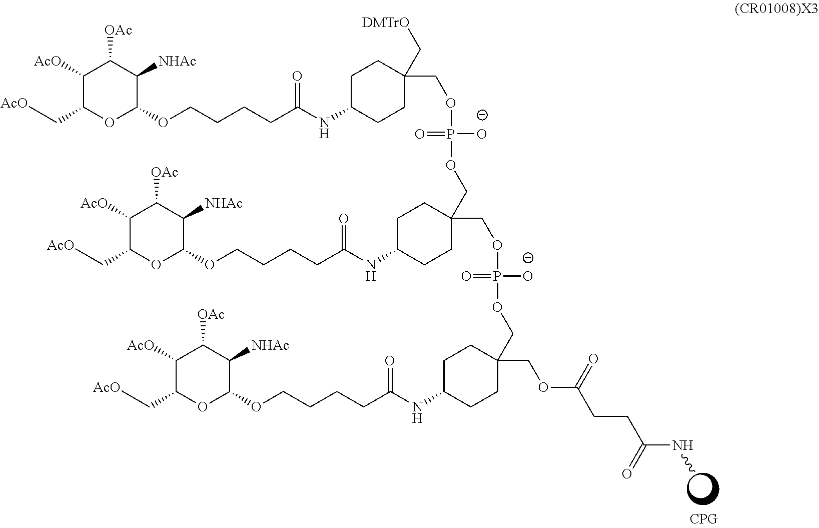

### (3.2) Synthesis of Antisense Strand (AS)

The antisense strand was synthesized using a universal solid-phase carrier. Conditions of deprotection, coupling, capping, oxidation or sulfidation, conditions of cleavage and deprotection, and conditions of purification and desalting in the solid-phase synthesis method of the antisense strand were the same as those in the synthesis of the sense strand in step (3.1).

Detection: purity was detected using ion exchange chromatography (IEX-HPLC); and molecular weight was detected using liquid chromatography-mass spectrometry (LC-MS). An actually measured value and a theoretical value of the molecular weight were compared, and if the actually measured value and the theoretical value were consistent, it indicated that the antisense strand of the siRNA was obtained.

(3.3) Synthesis of siRNA Double Strands

The sense strand synthesized in step (3.1) and the antisense strand synthesized in step (3.2) were mixed in an equal molar ratio, dissolved in water for injection, heated to 95° C., slowly cooled to room temperature and kept at room temperature for 10 min, to make the sense strand and the antisense strand form a double-stranded structure via a hydrogen bond, thereby rendering the siRNA having the sense strand and the antisense strand as shown in TABLE 19.

In the above, when the carrier was three clusters of CR01008, a structural formula of the siRNA conjugate was as follows:

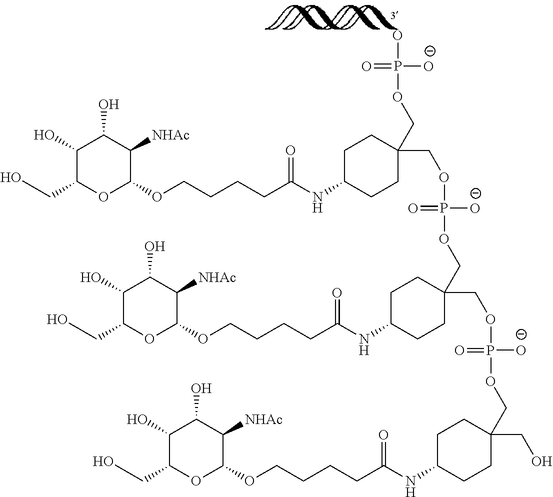

where

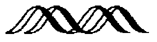

represents siRNA. (CR01018×3) carrier was conjugated to the 3′-end of the sense strand of siRNA.

TABLE 18
Information of Unmodified Nucleotide Sequences Forming siRNA
Conjugates

| | Sense Strand Sequence | | Antisense Strand Sequence | |
|---|---|---|---|---|
| No. | (5′-3′) | SEQ ID | (5′-3′) | SEQ ID |
| RZ597117 | CCAAGAGCACCAAGAAC | NO. 41 | UAGUUCUUGGUGCUCU | NO. 42 |
| | UA | | UGGCU | |
| RZ597118 | CCAAGAGCACCAAGAAC | NO. 41 | UAGUUCUUGGUGCUCU | NO. 42 |
| | UA | | UGGCU | |
| RZ597119 | CCAAGAGCACCAAGAAC | NO. 41 | UAGUUCUUGGUGCUCU | NO. 42 |
| | UA | | UGGCU | |
| RZ597120 | CCAAGAGCACCAAGAAC | NO. 41 | UAGUUCUUGGUGCUCU | NO. 42 |
| | UA | | UGGCU | |
| RZ597121 | CCAAGAGCACCAAGAAC | NO. 41 | UAGUUCUUGGUGCUCU | NO. 42 |
| | UA | | UGGCU | |
| RZ597122 | CCAAGAGCACCAAGAAC | NO. 41 | UAGUUCUUGGUGCUCU | NO. 42 |
| | UA | | UGGCU | |
| RZ597123 | CCAAGAGCACCAAGAAC | NO. 41 | UAGUUCUUGGUGCUCU | NO. 42 |
| | UA | | UGGCU | |
| RZ597124 | CCAAGAGCACCAAGAAC | NO. 41 | UAGUUCUUGGUGCUCU | NO. 42 |
| | UA | | UGGCU | |
| RZ597125 | CCAAGAGCACCAAGAAC | NO. 41 | UAGUUCUUGGUGCUCU | NO. 42 |
| | UA | | UGGCU | |
| RZ597126 | CCAAGAGCACCAAGAAC | NO. 41 | UAGUUCUUGGUGCUCU | NO. 42 |
| | UA | | UGGCU | |
| RZ597127 | CCAAGAGCACCAAGAAC | NO. 41 | UAGUUCUUGGUGCUCU | NO. 42 |
| | UA | | UGGCU | |
| RZ597128 | CCAAGAGCACCAAGAAC | NO. 41 | UAGUUCUUGGUGCUCU | NO. 42 |
| | UA | | UGGCU | |

TABLE 19
Sequence Information of siRNA Conjugates

| | Sense Strand Sequence and | Antisense Strand Sequence and |
|---|---|---|
| No. | Modification (5′-3′) | Modification (5′-3′) |
| RZ597117 | CmsCmsAmAmGmAmGfCfAfCfCmAmAmG | UmsAfsGmUmUmCfUmUmGfG |
| | mAmAmCmUmAm(CR01008)(CR01008) | mUmGmCmUfC(moe)UfUmGm |
| | (CR01008) (SEQ ID: 60) | GmsCmsUm (SEQ ID: 61) |
| RZ597118 | CmsCmsAmAmGmAmGfCfAfCfCmAmAmG | UmsAfsG(TBDMS)UmUmCfU |
| | mAmAmCmUmAm(CR01008)(CR01008) | mUmGfGmUmGmCmUfC(moe) |
| | (CR01008) (SEQ ID: 60) | UfUmGmGmsCmsUm (SEQ ID: |
| | | 62) |
| RZ597119 | CmsCmsAmAmGmAmGfCfAfCfCmAmAmG | UmsAfsGmU(TBDMS)UmCfU |
| | mAmAmCmUmAm(CR01008)(CR01008) | mUmGfGmUmGmCmUfC(moe) |
| | (CR01008) (SEQ ID: 60) | UfUmGmGmsCmsUm (SEQ ID: |
| | | 63) |
| RZ597120 | CmsCmsAmAmGmAmGfCfAfCfCmAmAmG | UmsAfsGmUmU(TBDMS)CfU |
| | mAmAmCmUmAm(CR01008)(CR01008) | mUmGfGmUmGmCmUfC(moe) |
| | (CR01008) (SEQ ID: 60) | UfUmGmGmsCmsUm (SEQ ID: |
| | | 64) |
| RZ597121 | CmsCmsAmAmGmAmGfCfAfCfCmAmAmG | UmsAfsGmUmUmC(TBDMS)U |
| | mAmAmCmUmAm(CR01008)(CR01008) | mUmGfGmUmGmCmUfC(moe) |
| | (CR01008) (SEQ ID: 60) | UfUmGmGmsCmsUm (SEQ ID: |
| | | 65) |
| RZ597122 | CmsCmsAmAmGmAmGfCfAfCfCmAmAmG | UmsAfsGmUmUmCfU(TBDMS) |
| | mAmAmCmUmAm(CR01008)(CR01008) | UmGfGmUmGmCmUfC(moe) |
| | (CR01008) (SEQ ID: 60) | UfUmGmGmsCmsUm (SEQ ID: |
| | | 66) |
| RZ597123 | CmsCmsAmAmGmAmGfCfAfCfCmAmAmG | UmsAfsGmUmUmCfUmU(TBDMS) |
| | mAmAmCmUmAm(CR01008)(CR01008) | GfGmUmGmCmUfC(moe)U |
| | (CR01008) (SEQ ID: 60) | fUmGmGmsCmsUm (SEQ ID: |
| | | 67) |
| RZ597124 | CmsCmsAmAmGmAmGfCfAfCfCmAmAmG | UmsAfsGmUmUmCfUmUmG |
| | mAmAmCmUmAm(CR01008)(CR01008) | (TBDMS)GmUmGmCmUfC(moe) |
| | (CR01008) (SEQ ID: 60) | UfUmGmGmsCmsUm (SEQ ID: |
| | | 68) |
| RZ597125 | CmsCmsAmAmGmAmGfCfAfCfCmAmAmG | UmsAfsGmU(TOM)UmCfUmU |
| | mAmAmCmUmAm(CR01008)(CR01008) | mGfGmUmGmCmUfC(moe)Uf |
| | (CR01008) (SEQ ID: 60) | UmGmGmsCmsUm (SEQ ID: |
| | | 69) |
| RZ597126 | CmsCmsAmAmGmAmGfCfAfCfCmAmAmG | UmsAfsGmUmU(TOM)CfUmU |
| | mAmAmCmUmAm(CR01008)(CR01008) | mGfGmUmGmCmUfC(moe)Uf |
| | (CR01008) (SEQ ID: 60) | UmGmGmsCmsUm (SEQ ID: |
| | | 70) |
| RZ597127 | CmsCmsAmAmGmAmGfCfAfCfCmAmAmG | UmsAfsGmUmUmCfU(TOM)U |
| | mAmAmCmUmAm(CR01008)(CR01008) | mGfGmUmGmCmUfC(moe)Uf |
| | (CR01008) (SEQ ID: 60) | UmGmGmsCmsUm (SEQ ID: |
| | | 71) |
| RZ597128 | CmsCmsAmAmGmAmGfCfAfCfCmAmAmG | UmsAfsGmUmUmCfUmU(TOM) |
| | mAmAmCmUmAm(CR01008)(CR01008) | GfGmUmGmCmUfC(moe)UfU |
| | (CR01008) (SEQ ID: 60) | mGmGmsCmsUm (SEQ ID: 72) |

Unless otherwise noted, meanings of base composition and modifications used in the present disclosure are as follows: capital letters A, U, G, C, and T represent base composition of nucleotides; lowercase letter m means that a nucleotide adjacent to the left of the letter m is a 2′-O-methyl modified (also called: 2′-methoxy modified) nucleotide; lowercase letter f means that a nucleotide adjacent to the left of the letter f is a 2′-fluoro modified nucleotide; (moe) means that a nucleotide adjacent to the left of combined identity (moe) is a 2′-O-methoxyethyl (i.e., 2′-O-MOE) modified nucleotide; (TBDMS) means that a nucleotide adjacent to the left of combined identity (TBDMS) is a 2′-O-tert-butyldimethylsilyl (i.e., 2′-O-TBDMS) modified nucleotide; (TOM) means that a nucleotide adjacent to the left of combined identity (TOM) is a 2′-O-triisopropylmethoxysilane (i.e., 2′-O-TOM) modified nucleotide; and lowercase letter s means that two nucleotides adjacent to the left and right of the letter are linked by a phosphorothioate bond.

A structural formula of the 2′-O-methyl modified nucleotide was

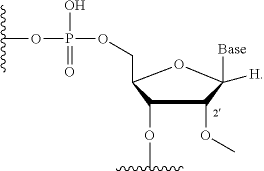

A structural formula of the 2′-fluoro modified nucleotide was

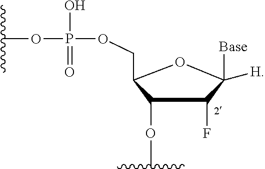

A structural formula of the 2′-O-MOE modified nucleotide was

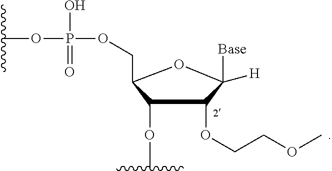

A structural of the 2′-O-TBDMS modified nucleotide was

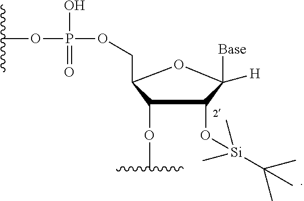

A structural formula of the 2′-O-TBDMS modified nucleotide was

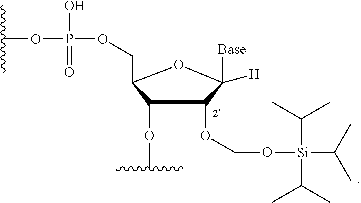

In the above, Base represents base of nucleotide, such as uracil U, thymine T, cytosine C, adenine A or guanine G.

TABLE 20
Detection Results of siRNA Conjugates

| | Sense Strand | | | Antisense Strand | | | siRNA Conjugate | | |
|---|---|---|---|---|---|---|---|---|---|
| siRNA | Theoretical | Actual | | Theoretical | Actual | | Theoretical | Actual | |
| Conjugate | Molecular | Molecular | Purity | Molecular | Molecular | Purity | Molecular | Molecular | Purity |
| No. | Weight | Weight | % | Weight | Weight | % | Weight | Weight | % |
| RZ597117 | 7896.9 | 7899.5 | 96.1 | 6963.52 | 6964.6 | 95.3 | 14860.42 | 14864.1 | 94 |
| RZ597118 | 7896.9 | 7899.5 | 96.1 | 7063.49 | 7065 | 95.8 | 14960.39 | 14964.5 | 91.6 |
| RZ597119 | 7896.9 | 7899.5 | 96.1 | 7063.49 | 7065.3 | 97.2 | 14960.39 | 14964.8 | 92.9 |
| RZ597120 | 7896.9 | 7899.5 | 96.1 | 7063.49 | 7065.3 | 97.2 | 14960.39 | 14964.8 | 92.2 |
| RZ597121 | 7896.9 | 7899.5 | 96.1 | 7075.52 | 7077.2 | 96.3 | 14972.42 | 14976.7 | 93.9 |
| RZ597122 | 7896.9 | 7899.5 | 96.1 | 7063.49 | 7065.3 | 96.6 | 14960.39 | 14964.8 | 93.8 |
| RZ597123 | 7896.9 | 7899.5 | 96.1 | 7063.49 | 7065.3 | 97.1 | 14960.39 | 14964.8 | 95.3 |
| RZ597124 | 7896.9 | 7899.5 | 96.1 | 7075.54 | 7077.1 | 96.4 | 14972.44 | 14976.6 | 93.1 |
| RZ597125 | 7896.9 | 7899.5 | 96.1 | 7135.52 | 7137.3 | 97.2 | 15032.42 | 15036.8 | 95.2 |
| RZ597126 | 7896.9 | 7899.5 | 96.1 | 7135.52 | 7137.3 | 97.2 | 15032.42 | 15036.8 | 94.2 |
| RZ597127 | 7896.9 | 7899.5 | 96.1 | 7135.52 | 7137.3 | 96.8 | 15032.42 | 15036.8 | 92 |
| RZ597128 | 7896.9 | 7899.5 | 96.1 | 7135.52 | 7137.3 | 95.7 | 15032.42 | 15036.8 | 93.9 |

As can be seen from data in TABLE 20, the sense strand (SS) and the antisense strand (AS) are able to be well linked to the ligand and maintain high purity.

### Biological Detection Experiments

Unless otherwise noted, experimental animals C57BL/6J mice used in the present disclosure were all purchased from SPF (Beijing) Biotechnology Co., Ltd.

Unless otherwise noted, reagents, consumable materials as well as instrument and equipment used in the present disclosure were all commercially available, where main reagent and consumable materials are shown in TABLE 21, and main instrument and equipment are shown in TABLE 22.

TABLE 21
Main Reagents and Consumable Materials

| Name | Manufacturer |
|---|---|
| DMEM culture medium | M&C GENE TECHNOLOGY(BEIJING) LTD. |
| Fetal bovine serum(FBS) | Merck |
| Bispecific antibody | BBI |
| 1×PBS | M&C GENE TECHNOLOGY(BEIJING) LTD. |
| Hanwei RNA extraction kit | Zhejiang Hanwei Science and Technology Co. Ltd. |
| Reverse Transcription System | Promega Corporation |
| SYBR Select Master Mix | ABI |
| HBSS | M&C GENE TECHNOLOGY(BEIJING) LTD. |
| EDTA | Solarbio |
| Type IV collagenase | Sigma |
| RNALater | Thermo Fisher Scientific |

TABLE 22
Main Instrument and Equipment

| Name | Manufacturer |
|---|---|
| Thermostatic water bath | Shanghai Boxun |
| Automated cell counter | Shanghai Countstar Inc. |
| Inverted microscope | Olympus |
| Biological safety cabinet | Esco Lifesciences |
| Cell incubator | Thermo Fisher Scientific |
| Full-automatic Nucleic Acid Extractor | Zhejiang Hanwei Science and Technology Co. |
| | Ltd. |
| High speed refrigerated centrifuge | Eppendorf |
| NANODROP OneC | Thermo Fisher Scientific |
| Gradient PCR amplifier | Eppendorf |
| Fluorescence quantitative PCR instrument | ABI StepOne Plus |
| Gel imager | Shanghai Tanon Science & Technology Co., Ltd. |
| Electrophoresis apparatus | BEIJING LIUYI INSTRUMENT PLANT |
| Tissuelyser II full-automatic tissue | Shanghai Jingxin Industrial Development Co., |
| homogenizer | Ltd. |

Evaluation Method for Inhibitory Activity of siRNA Conjugates on Target Genes in Mouse Primary Livers

### Isolation of Mouse Primary Hepatocytes:

Mouse primary hepatocytes were extracted from C56BL/6j mouse fresh liver tissues. Specific operation steps were as follows: the mice were anesthetized by intraperitoneally injecting a 10% chloral hydrate solution, and the mice were fixed and disinfected with 75% ethanol for abdomens and chests thereof. Surgical instrument was sterilized to open abdomens and expose portal veins and inferior vena cavas. Indwelling needle was installed with a heparin cap, a scalp needle that had been connected to an infusion pump transfusion bottle (0.5 mM EDTA HBSS perfusion fluid) was accessed. The needle was introduced from the inferior vena cavas, and perfusion was carried out at a rate of 120 drops/min. The hepatic portal veins were cut, to make perfusate flow out from the cut hepatic portal veins. The perfusion was carried out for 4 min, and subsequently, the perfusion was changed into 0.8 mg/mL type IV collagenase HBSS solution (Sigma, C5138) (containing 0.08% DNAI enzyme (sigma, DN25)) and further perfused for 8 min. The perfused livers were taken out from the animals, washed with HBSS (containing Ca2+, Mg2+, MACGENE, CC016), placed in a sterile culture vessel, and shredded with addition of DMEM complete culture medium (DMEM culture medium+10% serum). Cell suspension was filtered by cytoscreener to remove undigested tissues and connective tissues, supernatant was discarded by centrifugation at 800 rpm for 3 min, and DMEM complete culture medium was added again, followed by suspension and centrifugation, to render primary mouse hepatocytes. Cell culturing and transfection:

DMEM complete culture medium was added to adjust cell density to 2×10⁵ cells/mL and render a mouse primary hepatocyte suspension. Subsequently, the cells were inoculated into a 12-well culture plate coated with mouse tail collagen type I in advance (coating was performed at a concentration of 2 μg/cm² by a coating method as described in solarbio (C8062) instructions), and a volume of the cell suspension added was 1000 μL/well, that is, a cell amount was 2×10⁵ cells/well.

Various groups of conjugates were diluted with PBS into working solutions at a concentration 500 times final test concentration value (based on siRNA). The siRNA conjugate working solutions were added into the 12-well culture plate, with 2 μL/well, and each siRNA conjugate was set with 2 culture wells. Another 2-3 culture wells were added with PBS as blank control wells, with 2 μL/well. The culture plate was shaken to mix solutions uniformly. The culture plate was further cultured in a 37° C., 5% CO₂ cell incubator for 24 h.

Detection of mRNA Expression Level:

RNA extraction: total RNAs of various groups of primary hepatocyte samples were extracted using full-automatic nucleic acid extraction instrument and a nucleic acid extraction kit from Zhejiang Hanwei Science and Technology Co. Ltd. according to a method described in instructions. Reverse transcription reaction: 1000 ng of the extracted total RNAs of the primary hepatocyte samples were taken, and configured into 20 μL of reverse transcription system according to a method described in kit instructions by using a reverse transcription kit (Reverse Transcription System, A3500) from Promega company and selecting Oligo (dT) 15 reverse transcription primer, and the reverse transcription reaction was completed. After the reaction was ended, 80 μL of RNase-Free water was added into the reverse transcription system to render cDNA solution for Real-time PCR detection. Real-time PCR detection: 20 μL of Real-time PCR reaction system per PCR detection well was configured according to a method described in kit instructions by using SYBR™ Select Master Mix (Catalog number: 4472908) reagent from ABI company. Each detection system contained 5 μL of cDNA template obtained from the above reverse transcription reaction, 10 μL of SYBR™ Select Master Mix, 0.5 μL of 10 μM upstream primers, 0.5 μL of 10 μM downstream primers (see TABLE 23 for primer information), and 4 μL of RNase-Free H₂O. The configured reaction system was subjected to Real-time PCR amplification on ABI StepOnePlus PCR instrument by a three-step method, wherein an amplification procedure was pre-denaturation at 95° C. for 10 min, then denaturation at 95° C. for 30 s, annealing at 60° C. for 30 s, and extension at 72° C. for 30 s, and processes of denaturation, annealing and extension were repeated for 40 cycles.

In the real-time fluorescence quantitative PCR method, relative quantitative calculation of remaining expression level and inhibitory rate of gene mRNA of interest in various test groups was performed by a ΔΔCt method, and the calculation method is as follows:

$$Δ Ct ( test group ) = Ct ( gene of interest in test group ) - Ct ( reference gene in test group ) ;$$

$$Δ Ct ( control group ) = Ct ( gene of interest in control group ) - Ct ( reference gene in control group ) ;$$

$$ΔΔ Ct ( test group ) = Δ Ct ( test group ) - Δ Ct ( control group mean ) ;$$

$$ΔΔ Ct ( control group ) = Δ Ct ( control group ) - Δ Ct ( control group mean ) .$$

In the above, ΔCt (control group mean) in cell experiments was an arithmetic mean value of ΔCt (control group) of several replicate wells in the control group. Thus, each cell well in the test group and the control group is corresponding to one ΔΔCt value.

Taking the control group as reference, expression level of the gene mRNA of interest in the test group was normalized, and remaining expression level of the gene mRNA of interest in the control group was defined as 100%.

$$Relative remaining expression level of gene mRNA of interest in test group = 2 - ΔΔ Ct ( test group ) \times 100 \%$$

$$Inhibitory rate of expression of gene mRNA of interest in test group = ( 1 - relative remaining expression level of gene mRNA of interest in test group \times 100 \%$$

Unless otherwise noted, activity experimental data are all expressed by x̅+SD, and experimental data are all plotted and analyzed using GraphPad prism 8.0 software.

### Example 12 In Vitro Inhibitory Activity of siRNA Conjugates with TBDMS Modification at Different Sites of Antisense Strand on ANGPTL3 mRNA in Mouse Primary Hepatocytes

Example 12 evaluated the inhibitory activity of siRNA conjugates with TBDMS modification at different sites of antisense strand on target gene ANGPTL3 of interest in mouse primary hepatocytes by the above evaluation method for inhibitory activity on a target gene in mouse primary hepatocytes. Isolation of mouse primary hepatocytes, cell culturing and other operations were as described in the preceding. Mouse primary hepatocytes were extracted from C56BL/6j mouse fresh liver tissues, inoculated in a 12-well culture plate at a cell amount of 2×10⁵ cells/well. Various groups of double-stranded siRNA conjugates were gradiently diluted with PBS to 5 μM working solutions (based on siRNA). 2 μL of the working solutions of siRNA conjugates at each concentration were respectively added into the above 12-well culture plate per well, equivalent to that a siRNA conjugate transfection final concentration was 10 nM (based on siRNA), and siRNA at each concentration was set with 2 culture wells. Another 2 culture wells were added with 2 μL of PBS per well as blank control wells (BLANK). The culture plate was shaken to mix solutions uniformly. The culture plate was further cultured in a 37° C., 5% CO₂ cell incubator for 24 h. Relative quantitative calculation for the gene mRNA of interest in various test groups was performed by the preceding ΔΔCt method.

TABLE 23
Information of Primer Sequences in Example 12

| Target Point | | Primer Type | Primer Sequence (5′-3′) |
|---|---|---|---|
| Gene of | ANGPTL3 | Upstream | GAGGAGCAGCTAACCAACTTAAT (SEQ ID |
| Interest | | Primer | NO: 51) |
| | | Downstream | TCTGCATGTGCTGTTGACTTAAT (SEQ ID |
| | | Primer | NO: 52) |
| Reference | GAPDH | Upstream | TGCACCACCAACTGCTTAG (SEQ ID NO: 53) |
| Gene | | Primer | |
| | | Downstream | GGATGCAGGGATGATGTTC (SEQ ID NO: 54) |
| | | Primer | |

TABLE 24
Inhibitory Activity of siRNA Conjugates
on Target Gene ANGPTL3 mRNA

| Group | Mean Value of Inhibitory Rate/% | SD Value |
|---|---|---|
| BLANK | 0.00 | 10.56 |
| RZ597117 | 87.31 | 2.50 |
| RZ597118 | 40.68 | 6.24 |
| RZ597119 | 51.38 | 6.68 |
| RZ597120 | 69.48 | 0.41 |
| RZ597121 | 88.01 | 2.37 |
| RZ597122 | 89.37 | 0.34 |
| RZ597123 | 87.00 | 3.58 |
| RZ597124 | 91.79 | 0.77 |

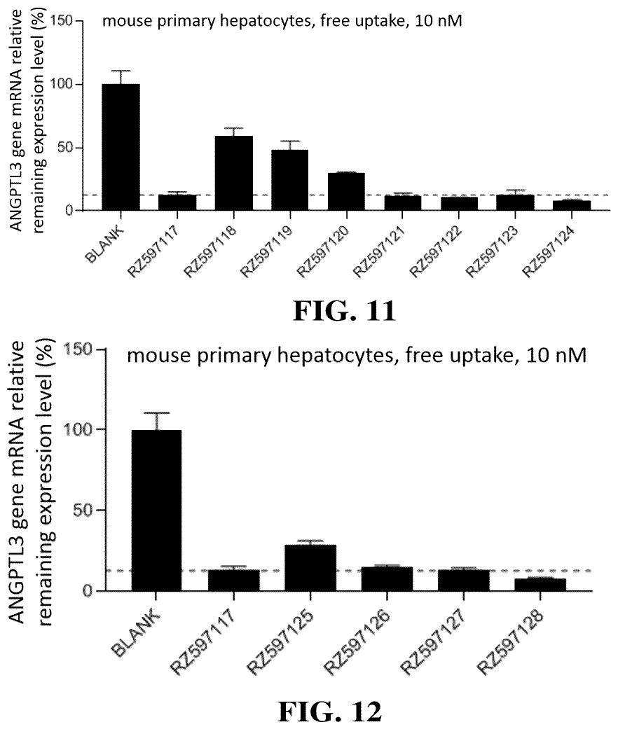

Results of Example 12 are as shown in FIG. 11 and TABLE 24. TBDMS modified sequences with TBDMS modification at site 6 (RZ597121), site 7 (RZ597122), site 8 (RZ597123), site 9 (RZ597124) of siRNA antisense strand have comparable or superior in vitro inhibitory activity to the control sequence (RZ597117) without TBDMS modification.

### Example 13 In Vitro Inhibitory Activity of siRNA Conjugates with TOM Modification at Different Sites of Antisense Strand on ANGPTL3 mRNA in Mouse Primary Hepatocytes

Example 13 evaluated the inhibitory activity of siRNA conjugates with TOM modification at different sites of antisense strand on target gene ANGPTL3 of interest in mouse primary hepatocytes by the above experimental method for evaluation of inhibitory activity on a target gene in mouse primary hepatocytes.

Information of primer sequences used in Example 13 was the same as that in TABLE 2.3.

TABLE 25
Inhibitory Activity of siRNA Conjugates
on Target Gene ANGPTL3 mRNA.

| Group | Mean Value of Inhibitory Rate/% | SD Value |
|---|---|---|
| BLANK | 0.00 | 10.56 |
| RZ597117 | 87.31 | 2.50 |
| RZ597125 | 71.65 | 2.89 |
| RZ597126 | 85.48 | 1.55 |
| RZ597127 | 87.23 | 1.55 |
| RZ597128 | 92.57 | 0.98 |

Results of Example 13 are as shown in FIG. 12 and TABLE 25. TOM modified sequences with TOM modification at site 5 (RZ597126), site 7 (RZ597127), and site 8 (RZ597128) of siRNA antisense strand have comparable or superior in vitro activity to the control sequence (RZ597117) without TOM modification.

Finally, it should be noted that various examples above are merely used for illustrating the technical solutions of the present disclosure, rather than limiting the present disclosure. Although the detailed description is made to the present disclosure with reference to various preceding examples, those ordinarily skilled in the art should understand that they still could modify the technical solutions described in various preceding examples, or make equivalent substitutions to some or all of the technical features therein. These modifications or substitutions do not make the corresponding technical solutions essentially depart from the scope of the technical solutions of various examples of the present disclosure.

### INDUSTRIAL APPLICABILITY

In order to reduce the off-target effect of nucleic acid molecule of dsRNA drugs as much as possible while maintaining good pharmacodynamic activity of this type of drugs, the present disclosure adopts a novel modification strategy, that is, using a nucleotide N_{M} modified with a bulky group that does not attenuate the Tm value at a specific position of the antisense strand of dsRNA, can significantly reduce the off-target effect of the siRNA sequence, and at the same time maintain the good pharmacodynamic activity. Specifically, the modified double-stranded oligonucleotide provided by the present disclosure contains the sense strand and the antisense strand, wherein sites 1-9 of the antisense strand counting from the 5′-end contain at least one nucleotide N_{M}, the nucleotide N_{M} being a nucleotide modified with 2′-Si-containing group (wherein the 2′-Si-containing group has a greater steric hindrance than 2′-methoxy modification). The present disclosure found that the dsRNA containing at least one nucleotide N_{M} in a region of the sites 1-9 of the antisense strand counting from the 5′-end is more effective in reducing the off-target effect than the parental dsRNA molecule lacking corresponding modification.

When the double-stranded oligonucleotide inhibiting target gene expression provided by the present disclosure is applied to a method for preparing a pharmaceutical composition or inhibiting target gene expression, since the dsRNA can effectively inhibit target gene expression, and the anti-off-target effect thereof is more excellent, drugs containing the above dsRNA molecule can effectively reduce adverse effects of drugs caused by off-target.

According to some aspects of the present disclosure, while maintaining good pharmacodynamic activity and liver delivery efficiency, the double-stranded oligonucleotide conjugate of the present disclosure has a lower nucleic acid molecule off-target effect than the corresponding oligonucleotide molecule lacking corresponding modification, and can effectively reduce toxic and side effects of drugs caused by off-target.

1. A modified double-stranded oligonucleotide molecule, which contains a sense strand and an antisense strand, wherein nucleotide sequences of the sense strand and the antisense strand are at least partially reversely complementary, wherein positions of sites 1-9 of the antisense strand, counting from a 5′-end, contain at least one modified nucleotide N_{M}, the nucleotide N_{M} being a nucleotide modified with 2′-Si-containing group; and in the modified nucleotide, the 2′-Si-containing group has a steric bulk greater than that of 2′-methoxy group.

2. The double-stranded oligonucleotide molecule according to claim 1, wherein the nucleotide N_{M} is a nucleotide modified with a 2′—O—Si— group;
alternatively, the nucleotide N_{M} has a structure as represented by following Formula (I), or is a tautomer of Formula (I): 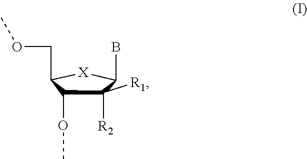
where X is selected from the group consisting of CH₂, O, S and NH;
B is selected from the group consisting of nucleotide bases, heteroaryls having 6-12 carbon atoms, heterocyclic bases and base substitution groups;
the nucleotide bases are selected from the group consisting of: uracil or derivatives thereof, thymine or derivatives thereof, cytosine or derivatives thereof, 5-methylcytosine or derivatives thereof, adenine or derivatives thereof, and guanine or derivatives thereof; and
R₁ and R₂ are each independently selected from H or —(O)ₘ(CH₂)ₙOR₃, and R₁ and R₂ are not both H or —(O)ₘ(CH₂)ₙOR₃; m is selected from 0 or 1, and n is an integer selected from 0 to 3, where —(O)ₘ(CH₂)ₙOR₃ substituent has a steric bulk greater than that of methoxy, and R₃ is a Si-containing group.

3. The double-stranded oligonucleotide molecule according to claim 1, wherein the nucleotide N_{M} has a structure as represented by following Formula (II), or is a tautomer of Formula (II): 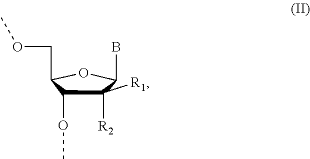
where B is selected from the group consisting of uracil or derivatives thereof, thymine or derivatives thereof, cytosine or derivatives thereof, 5-methylcytosine or derivatives thereof, adenine or derivatives thereof, and guanine or derivatives thereof; and
R₁ and R₂ are each independently selected from H or —(O)ₘ(CH₂)ₙOR₃, and R₁ and R₂ are not both H or —(O)ₘ(CH₂)ₙOR₃; m is selected from 0 or 1, and n is an integer selected from 0 to 2, where —(O)ₘ(CH₂)ₙOR₃ substituent has a steric bulk greater than that of methoxy,
wherein R₃ is —Si(R₄)₃, where each R₄ is independently selected from the group consisting of: substituted or unsubstituted C1-C6 alkyl, and substituted or unsubstituted C1-C6 alkoxy, wherein a substituent is selected from the group consisting of C1-C6 alkyl, hydroxy, halogen, alkoxy having no more than 6 carbon atoms, amino, cycloalkyl having no more than 12 carbon atoms, aryl having no more than 12 carbon atoms and heteroaryl having no more than 12 carbon atoms;
optionally, n is 0 or 1;
optionally, R₁ or R₂ is a —O-TOM group, and R₁ and R₂ are not both —O-TOM group;
optionally, the nucleotide N_{M} has a structure as represented by following Formula (III), or is a tautomer of Formula (III): 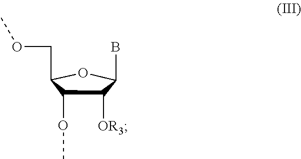
B is selected from the group consisting of uracil, thymine, cytosine, 5-methylcytosine, adenine and guanine;
R₃ is —Si(R₄)₃, where each R₄ is independently selected from the group consisting of:
substituted or unsubstituted C1-C6 alkyl, and substituted or unsubstituted C1-C6 alkoxy, wherein a substituent is selected from the group consisting of C1-C6 alkyl, hydroxy, halogen, alkoxy having no more than 6 carbon atoms and amino; and
optionally, R₃ is selected from TBDMS group or TIPS group.

4. The double-stranded oligonucleotide molecule according to claim 1, wherein in addition to the modified nucleotide N_{M}, the double-stranded oligonucleotide molecule further contains other modified nucleotides,
wherein the other modified nucleotides are at least one selected from the group consisting of: abasic nucleotides, reverse abasic nucleotides, reverse deoxyribonucleotides, 2′-halogen modified nucleotides, 2′-methoxy modified nucleotides, 2′-deoxy modified nucleotides, 2′-O-methoxyalkyl modified nucleotides, 2′-O-alkyl modified nucleotides, 2′-O-allyl modified nucleotides, 5′-vinyl modified nucleotides, 5′-cyclopropyl modified nucleotides, BNA, LNA, 5′-vinylphosphate modified nucleotides, 5′-cyclopropylphosphate modified nucleotides and 5′-alkyl substituent phosphate modified nucleotides;
optionally, the other modified nucleotides are at least two selected from the group consisting of 2′-methoxy modified nucleotides, 2′-halogen modified nucleotides and 2′-deoxy modified nucleotides;
optionally, the sense strand and the antisense strand both contain 2′-methoxy modified nucleotide and 2′-halogen modified nucleotide;
optionally, the sense strand and the antisense strand each independently contain no more than four 2′-fluoro modified nucleotides; and no less than five 2′-methoxy modified nucleotides;
optionally, the sense strand contains 14-40 nucleotides, optionally 14-30 nucleotides, optionally 17-25 nucleotides, and optionally 17-23 nucleotides;
optionally, the antisense strand contains 14-40 nucleotides, optionally 14-30 nucleotides, optionally 17-25 nucleotides, and optionally 19-23 nucleotides;
optionally, the double-stranded oligonucleotide molecule contains a duplex region of 19-21 base pairs; and
optionally, at least one linkage in the double-stranded oligonucleotide molecule is modified, wherein modification of the linkage comprises at least one of phosphorothioate internucleotide linkage modification or methylphosphonate internucleotide linkage modification.

5. The double-stranded oligonucleotide molecule according to claim 1, wherein the double-stranded oligonucleotide molecule further can contain a ligand;
optionally, the ligand is selected from the group consisting of galactose, galactosamine, N-acetylgalactosamine and derivatives thereof;
optionally, the ligand is covalently attached to a 5′-end or a 3′-end of the sense strand of the double-stranded oligonucleotide molecule by a linker;
optionally, a 3′-end and/or 5′-end of the sense strand and the antisense strand contains one or more overhang regions and/or capping groups; and
optionally, the overhang region contains 1-6 nucleotides.

6. The double-stranded oligonucleotide molecule according to claim 1, wherein the double-stranded oligonucleotide molecule is as represented by following Formula (A), and the double-stranded oligonucleotide molecule contains an antisense strand represented by Formula A2 and a sense strand represented by Formula A1:
5′-(N_{A})ₓ—(N_{L})ₛ—(N_{L}2)_{y}-3′; Formula A1:
3′-(N_{B})_{z}—(N_{L})ₜ—[N_{L}(N_{M})]₈—N_{L1}-5′(A), Formula A2:
where x is an integer selected from 0 to 6; s is an integer selected from 10 to 40; y is an integer selected from 0 to 3; z is an integer selected from 0 to 6; t is an integer selected from 10 to 30; and (N_{L}2)_{y} is an overhang nucleotide that does not undergo base complementary pairing with a nucleotide in the antisense strand;
each nucleotide N_{L} is independently an unmodified nucleotide or selected from following modified nucleotides: abasic nucleotides, reverse abasic nucleotides, reverse deoxyribonucleotides, 2′-halogen modified nucleotides, 2′-O-methoxyalkyl modified nucleotides, 2′-O-alkyl modified nucleotides, 2′-O-allyl modified nucleotides, 5′-vinyl modified nucleotides, 5′-cyclopropyl modified nucleotides, BNA, LNA or 2′-deoxy modified nucleotides;
[N_{L}(N_{M})₈ is eight contiguous nucleotides composed of the nucleotide N_{L} and the nucleotide N_{M} located at sites 2-9 of the antisense strand from the 5′-end, and contains at least one nucleotide N_{M} therein; the nucleotide N_{M} is located at any position of sites 2-9 of the antisense strand from the 5′-end; and each nucleotide N_{M} is independently selected from above Formula (I), Formula (II) or Formula (III);
each nucleotide N_{A} is independently an unmodified nucleotide or selected from following modified nucleotides: abasic nucleotides, reverse abasic nucleotides, reverse deoxyribonucleotides, 2′-halogen modified nucleotides, 2′-O-methoxyalkyl modified nucleotides, 2′-O-methyl modified nucleotides, 2′-O-allyl modified nucleotides or 2′-deoxy modified nucleotides;
each nucleotide N_{B} is independently an unmodified nucleotide or selected from following modified nucleotides: abasic nucleotides, reverse abasic nucleotides, reverse deoxyribonucleotides, 2′-halogen modified nucleotides, 2′-O-methoxyalkyl modified nucleotides, 2′-O-methyl modified nucleotides, 2′-O-allyl modified nucleotides or 2′-deoxy modified nucleotides;
the nucleotide Nui is selected from nucleotides defined by the nucleotide N_{L}, or is selected from following nucleotides: 5′-vinylphosphate modified nucleotides, 5′-cyclopropylphosphate modified nucleotides or 5′-alkyl substituent phosphate modified nucleotides;
each nucleotide N_{L}.2 is independently an unmodified nucleotide or selected from following modified nucleotides: abasic nucleotides, reverse abasic nucleotides, reverse deoxyribonucleotides, 2′-halogen modified nucleotides, 2′-O-methoxyalkyl modified nucleotides, 2′-O-alkyl modified nucleotides, 2′-O-allyl modified nucleotides, 2′-vinyl modified nucleotides, 2′-cyclopropyl modified nucleotides, BNA, LNA or 2′-deoxy modified nucleotides;
optionally, in the [N_{L}(N_{M})]₈, the nucleotide N_{M} is located at any position of sites 3-8 of the antisense strand counting from the 5′-end;
optionally, when the nucleotide N_{L} is a modified nucleotide, each nucleotide N_{L1} is independently selected from following modified nucleotides: 2′-methoxy modified nucleotides, 2′-fluoro modified nucleotides or 2′-deoxy modified nucleotides;
optionally, s is an integer selected from 10 to 25, and t is an integer selected from 10 to 16;
optionally, each nucleotide N_{L} in the [N_{L}(N_{M})]₈ is independently selected from following modified nucleotides: 2′-methoxy modified nucleotides, 2′-fluoro modified nucleotides or 2′-deoxy modified nucleotides; and each nucleotide N_{M} is independently selected from nucleotides represented by the Formula (II) or the Formula (III);
optionally, the double-stranded oligonucleotide molecule further has at least one of following characteristics:
(i) a melting temperature Tm value that is not significantly reduced;
(ii) the sense strand and the antisense strand each independently containing no more than four 2′-halogen modified nucleotides, optionally, the sense strand and the antisense strand each independently containing no more than four 2′-fluoro modified nucleotides; and optionally, the antisense strand containing no more than four 2′-fluoro modified nucleotides, and the sense strand containing no more than three 2′-fluoro modified nucleotides;
(iii) the sense strand and the antisense strand each independently containing 1-4 phosphorothioate internucleotide linkages, optionally, the sense strand and the antisense strand each independently containing at least two phosphorothioate internucleotide linkages;
(iv) the sense strand and the antisense strand each independently containing at least two 2′-methoxy modifications, optionally, the sense strand and the antisense strand each independently containing at least three 2′-methoxy modifications, optionally, containing at least five 2′-methoxy modifications; and
(v) the double-stranded oligonucleotide molecule containing a duplex region having a length of 17-25 nucleotide pairs.

7. The double-stranded oligonucleotide molecule according to claim 6, wherein the modified double-stranded oligonucleotide molecule contains a sense strand as represented by Formula A1b and an antisense strand as represented by Formula A2b, wherein each strand has a length of 14-30 nucleotides, the antisense strand contains a sequence complementary to a target gene sequence, the sense strand contains a sequence sufficiently complementary to a sequence of the antisense strand so as to form a duplex region, and the modified double-stranded oligonucleotide molecule is as represented by following Formula (β):
5′-(N_{A})ₓ—(N_{L})ₛ—(N_{L}2)_{y}-3′; Formula A1b:
3′-(N_{B})_{z}—(N_{L})ₜ—[(N_{L})ᵢ—N_{M}—(N_{L})₇₋ᵢ]₈—N_{L1}-5′ Formula A2b: Formula (β),
where x and z are each independently an integer selected from 0 to 6, s is an integer selected from 10 to 25, y is an integer selected from 0 to 3, t is an integer selected from 9 to 16, and i is an integer selected from 0 to 7; each nucleotide N_{L}, each nucleotide N_{L1} and each nucleotide N_{L}2 are independently selected from unmodified nucleotides or from following modified nucleotides: 2′-deoxy modified nucleotides, abasic nucleotides, 2′-fluoro modified nucleotides or 2′-O-methyl modified nucleotides, wherein each nucleotide N_{A} and each nucleotide N_{B} are independently selected from unmodified nucleotides or from following modified nucleotides:
abasic nucleotides, 2′-fluoro modified nucleotides, 2′-O-methyl modified nucleotides or 2′-deoxy modified nucleotides;
optionally, at least one nucleotide N_{B} on the antisense strand is linked to a neighboring nucleotide by a phosphorothioate linkage;
optionally, x is an integer selected from 0 to 3, s is an integer selected from 14 to 23, z is an integer selected from 1 to 3, t is an integer selected from 10 to 16, and i is an integer selected from 1 to 6;
further optionally, in the double-stranded oligonucleotide molecule, the sense strand or the antisense strand each have following characteristics:
(a) the sense strand has one or more or all of following characteristics (i) to (iv):
(i) a length of 19-21 nucleotides; (ii) a ligand attached to a 3′-end, the ligand comprising a GalNAc derivative attached by a branched linker; (iii) no less than five 2′-OMe modifications and no more than five 2′-F modifications, and 0 or one deoxynucleotide; and, (iv) counting from a 5′-end of the sense strand, at least one phosphorothioate internucleotide linkage between positions 1-3 of nucleotide linking adjacent nucleotides;
(b) the antisense strand has one or more or all of following characteristics (i) to (iii):
(i) a length of 21-23 nucleotides, and no less than five 2′-OMe modifications and no more than five 2′-F modifications; (ii) at least one nucleotide N_{M} represented by the above Formula (II) or Formula (III) being present at sites 6-8 counting from a 5′-end; (iii) at least one phosphorothioate internucleotide linkage being present between positions 1-3 of nucleotide, and/or positions 21-23 of nucleotide counting from the 5′-end; and arbitrarily, the dsRNA having two nucleotide overhangs at a 3′-end of the antisense strand and a blunt end at the 5′-end of the antisense strand.

8. A pharmaceutical composition, which contains a therapeutically effective amount of the double-stranded oligonucleotide molecule according to claim 1 and a pharmaceutically acceptable adjuvant.

9. Use of the double-stranded oligonucleotide molecule according to claim 1 in a preparation of drugs for treating and/or preventing pathological conditions or diseases caused by expression of a specific gene.

10. A method for inhibiting target gene expression, wherein an effective amount of the double-stranded oligonucleotide molecule according to claim 1 is administered to a subject,
wherein the administration comprises subcutaneous or intravenous administration, and
wherein the subject is a mammal, optionally a human being.

11. A double-stranded oligonucleotide conjugate, wherein the double-stranded oligonucleotide conjugate contains a conjugate group and a double-stranded oligonucleotide molecule, the double-stranded oligonucleotide molecule contains a duplex formed by at least partial complementation of a sense strand and an antisense strand, and the sense strand and the antisense strand each have 17-35 nucleotides,
wherein sites 1-9 of the antisense strand counting from a 5′-end contain at least one nucleotide modified with 2′-(O)ₘ(CH₂)ₙOR₁ group, where m is selected from 0 or 1, n is an integer selected from 0 to 3, and R₁ is a Si-containing group; and optionally, n is selected from 0 or 1; and
the conjugate group is an asialoglycoprotein receptor (ASGPR) ligand group containing a galactosamine derivative unit; optionally, the galactosamine derivative is selected from the group consisting of galactosamine, N-formylgalactosamine, N-acetylgalactosamine, N-propionylgalactosamine, N-n-butyrylgalactosamine and N-isobutyrylgalactosamine.

12. The double-stranded oligonucleotide conjugate according to claim 11, wherein the double-stranded oligonucleotide conjugate has a structure as represented by following Formula (IV): 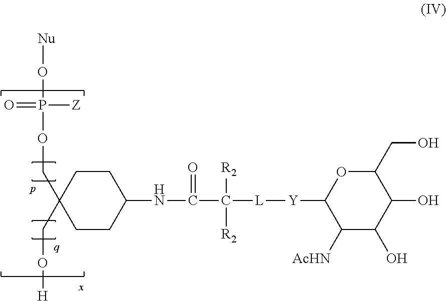
in Formula (IV), Nu represents the double-stranded oligonucleotide molecule;
x is 1, 2, 3 or 4;
each Z is independently hydroxyl or thiol;
each p is independently 1, 2 or 3;
each q is independently 1, 2 or 3;
each R₂ is independently H, an optionally substituted C₁-C₆ alkyl, a C₁-C₆ haloalkyl or an optionally substituted C₁-C₆ alkoxy;
L is selected from the group consisting of C₁-C₂₀ alkylene, —(O—CH₂—CH₂—)ⱼ—, C₁-C₂₀ alkylene substituted with one or more substituents of O, S, NH or —NH—C(O)—, and 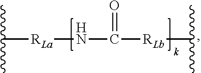
where R_{La} and R_{Lb} are independently selected from optionally substituted C₁₋₁₀ alkylene, k is selected from 1, 2 or 3, and j is an integer selected from 0 to 10; and
each Y is independently-NH—, O or S.

13. The double-stranded oligonucleotide conjugate according to claim 12, wherein L is 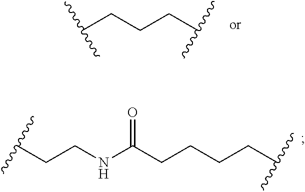 [SMILES: *CCC* ; *CCCCC(=O)NCC*]
selected from C₂-C₁₀ alkylene, and optionally, L selected from optionally, Y is O;
optionally, Z is hydroxyl; and
optionally, R₂ is H.

14. The double-stranded oligonucleotide conjugate according to claim 11, wherein the conjugate group has a structure as represented by following Formula V: 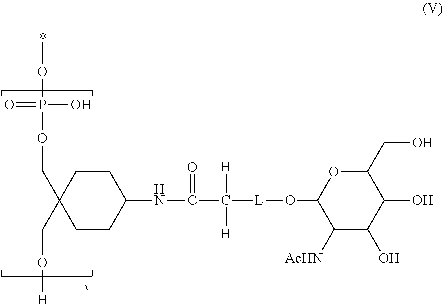
where * represents a covalent linking site of the conjugate group and the double-stranded oligonucleotide;
x is an integer selected from 1 to 3; and
L is an alkyl chain having 2 to 10 carbon atoms, and optionally, L is C₂-C₁₀ linear alkyl;
optionally, the conjugate group is any one selected from following structures, or a stereoisomer thereof, or a pharmaceutically acceptable salt thereof:

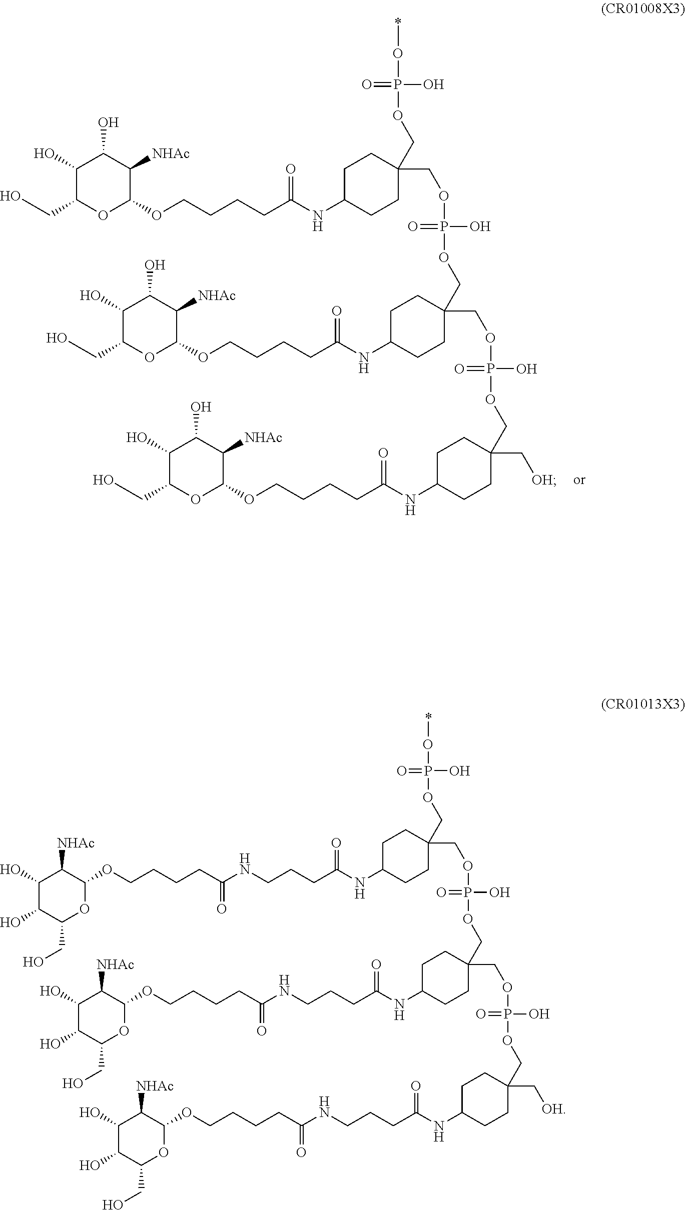

15. The double-stranded oligonucleotide conjugate according to claim 11, wherein R₁ is-Si(R₃)₃, where each R₃ is independently selected from the group consisting of substituted or unsubstituted C1-C6 alkyl, and substituted or unsubstituted C1-C6 alkoxy, wherein the substituent is selected from the group consisting of C1-C6 alkyl, hydroxy, halogen, amino and alkoxy having no more than 6 carbon atoms;
optionally, m and n are both 0;
optionally, m and n are both 1;
optionally, 2′-(O)ₘ(CH₂)ₙOR group is selected from 2′-O-TOM group, 2′-O-TBDMS group or 2′-O-TIPS group,
wherein a structural formula of TBDMS is as represented by following Formula (α), a structural formula of the TIPS group is as represented by following Formula (β), and a structural formula of the TOM group is as represented by following Formula (γ):

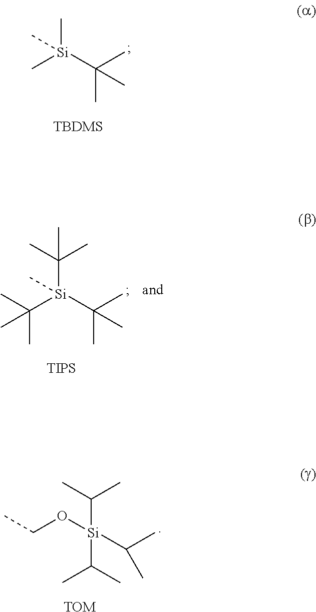

16. The double-stranded oligonucleotide conjugate according to claim 11, wherein each nucleotide in the duplex of the double-stranded oligonucleotide is modified;
optionally, in a direction from a 5′-end to a 3′-end, at least three nucleotides at sites 7-10 of the sense strand are 2′-fluoro modified nucleotides, and nucleotides at remaining positions are 2′-O-methyl modified nucleotides; and/or in a direction from a 5′-end to a 3′-end, at least one nucleotide at sites 2-9 of the antisense strand is selected from nucleotides modified with the 2′-(O)ₘ(CH₂)ₙOR₁ group; and
optionally, at least four nucleotides at sites 2, 6, 9, 12, 14 and 16 of the antisense strand are 2′-fluoro modified nucleotides, and nucleotides at remaining positions are 2′-O-methyl modified nucleotides; and/or nucleotide at site 15 is selected from 2′-O-methoxyethyl modified nucleotide or 2′-O-methyl modified nucleotide.

17. The double-stranded oligonucleotide molecule according to claim 11, wherein in a direction from a 5′-end to a 3′-end, at least three nucleotides at sites 7-10 of the sense strand are 2′-fluoro modified nucleotides, and nucleotides at remaining positions are independently selected from 2′-O-methyl modified nucleotides; and/or in a direction from a 5′-end to a 3′-end, at least one nucleotide at sites 3-8 of the antisense strand is selected from 2′-(O)ₘ(CH₂)ₙOR₁ group modified nucleotide, at least four of nucleotides at sites 2, 6, 9, 12, 14 and 16 are selected from 2′-fluoro modified nucleotides, nucleotide at site 15 is selected from 2′-O-methoxyethyl modified nucleotide or 2′-O-methyl modified nucleotide; and nucleotides at remaining positions are independently selected from 2′-O-methyl modified nucleotides;
optionally, in a direction from a 5′-end to a 3′-end, nucleotides at sites 7-10 of the sense strand are selected from 2′-fluoro modified nucleotides, and nucleotides at remaining positions are independently selected from 2′-O-methyl modified nucleotides; and in a direction from a 5′-end to a 3′-end, one nucleotide at sites 3-8 of nucleotide sequence of the antisense strand is selected from 2′-(O)ₘ(CH₂)ₙOR, group modified nucleotide, at least five of nucleotides at sites 2, 6, 12, 14 and 16 are selected from 2′-fluoro modified nucleotides, nucleotide at site 15 is selected from 2′-O-methoxyethyl modified nucleotide, and nucleotides at remaining positions are independently selected from 2′-O-methyl modified nucleotides.

18. A method for regulating specific gene expression in a target cell, wherein the method comprises:
making the double-stranded oligonucleotide conjugate according to claim 11 contact the target cell.

19. A method for preventing and/or treating diseases or conditions associated with dysregulated mRNA level of specific gene expression in a target cell in a subject, wherein the method comprises:
administering to the subject a pharmaceutically effective amount of the double-stranded oligonucleotide conjugate according to claim 11.

20. A kit, containing the double-stranded oligonucleotide conjugate according to claim 11.


SEQUENCE LISTING (74 sequences)

SEQ ID NO: 1 - RNA, 19 nt, synthetic construct
  source 1..19: mol_type = other RNA
  modified_base 1..6: mod_base = OTHER; note = 2'-methoxy
  modified_base 10..19: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 7: mod_base = OTHER; note = 2'-fluoro
  modified_base 9: mod_base = OTHER; note = 2'-fluoro
  modified_base 8: mod_base = OTHER; note = deoxyribonucleotide
ttctccgaacgtgtcacgt

SEQ ID NO: 2 - RNA, 19 nt, synthetic construct
  source 1..19: mol_type = other RNA
  modified_base 1..6: mod_base = OTHER; note = 2'-methoxy
  modified_base 10..19: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 7: mod_base = OTHER; note = 2'-fluoro
  modified_base 9: mod_base = OTHER; note = 2'-fluoro
  modified_base 8: mod_base = OTHER; note = deoxyribonucleotide
cagacagacaagaccatct

SEQ ID NO: 3 - RNA, 19 nt, synthetic construct
  source 1..19: mol_type = other RNA
  modified_base 1..6: mod_base = OTHER; note = 2'-methoxy
  modified_base 10..19: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 7: mod_base = OTHER; note = 2'-fluoro
  modified_base 9: mod_base = OTHER; note = 2'-fluoro
  modified_base 19: mod_base = OTHER; note = GalNA
  modified_base 8: mod_base = OTHER; note = deoxyribonucleotide
ccaagagcaccaagaacta

SEQ ID NO: 4 - RNA, 19 nt, synthetic construct
  source 1..19: mol_type = other RNA
  modified_base 1..6: mod_base = OTHER; note = 2'-methoxy
  modified_base 10..19: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 7: mod_base = OTHER; note = 2'-fluoro
  modified_base 9: mod_base = OTHER; note = 2'-fluoro
  modified_base 19: mod_base = OTHER; note = GalNA
  modified_base 8: mod_base = OTHER; note = deoxyribonucleotide
gtacgtggactggattctg

SEQ ID NO: 5 - RNA, 21 nt, synthetic construct
  source 1..21: mol_type = other RNA
  modified_base 1: mod_base = OTHER; note = 2'-methoxy
  modified_base 3..5: mod_base = OTHER; note = 2'-methoxy
  modified_base 7..13: mod_base = OTHER; note = 2'-methoxy
  modified_base 15: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 2: mod_base = OTHER; note = 2'-fluoro
  modified_base 6: mod_base = OTHER; note = 2'-fluoro
  modified_base 14: mod_base = OTHER; note = 14
  modified_base 16: mod_base = OTHER; note = 2'-fluoro
acgtgacacgttcggagaact

SEQ ID NO: 6 - RNA, 21 nt, synthetic construct
  source 1..21: mol_type = other RNA
  modified_base 1: mod_base = OTHER; note = 2'-methoxy
  modified_base 3..5: mod_base = OTHER; note = 2'-methoxy
  modified_base 7..13: mod_base = OTHER; note = 2'-methoxy
  modified_base 15: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 2: mod_base = OTHER; note = 2'-fluoro
  modified_base 6: mod_base = OTHER; note = 2'-fluoro
  modified_base 14: mod_base = OTHER; note = 2'-fluoro
  modified_base 16: mod_base = OTHER; note = 2'-fluoro
agatggtcttgtctgtctgga

SEQ ID NO: 7 - RNA, 21 nt, synthetic construct
  source 1..21: mol_type = other RNA
  modified_base 3..5: mod_base = OTHER; note = 2'-methoxy
  modified_base 7..13: mod_base = OTHER; note = 2'-methoxy
  modified_base 15: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 1: mod_base = OTHER; note = 2'-O-tert-butyldimethylsilyl
  modified_base 2: mod_base = OTHER; note = 2'-fluoro
  modified_base 6: mod_base = OTHER; note = 2'-fluoro
  modified_base 14: mod_base = OTHER; note = 2'-fluoro
  modified_base 16: mod_base = OTHER; note = 2'-fluoro
agatggtcttgtctgtctgga

SEQ ID NO: 8 - RNA, 21 nt, synthetic construct
  source 1..21: mol_type = other RNA
  modified_base 1: mod_base = OTHER; note = 2'-methoxy
  modified_base 3..5: mod_base = OTHER; note = 2'-methoxy
  modified_base 7..13: mod_base = OTHER; note = 2'-methoxy
  modified_base 15: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 2: mod_base = OTHER; note = 2'-O-tert-butyldimethylsilyl
  modified_base 6: mod_base = OTHER; note = 2'-fluoro
  modified_base 14: mod_base = OTHER; note = 2'-fluoro
  modified_base 16: mod_base = OTHER; note = 2'-fluoro
agatggtcttgtctgtctgga

SEQ ID NO: 9 - RNA, 21 nt, synthetic construct
  source 1..21: mol_type = other RNA
  modified_base 1: mod_base = OTHER; note = 2'-methoxy
  modified_base 4..5: mod_base = OTHER; note = 2'-methoxy
  modified_base 7..13: mod_base = OTHER; note = 2'-methoxy
  modified_base 15: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 3: mod_base = OTHER; note = 2'-O-tert-butyldimethylsilyl
  modified_base 2: mod_base = OTHER; note = 2'-fluoro
  modified_base 6: mod_base = OTHER; note = 2'-fluoro
  modified_base 14: mod_base = OTHER; note = 2'-fluoro
  modified_base 16: mod_base = OTHER; note = 2'-fluoro
agatggtcttgtctgtctgga

SEQ ID NO: 10 - RNA, 21 nt, synthetic construct
  source 1..21: mol_type = other RNA
  modified_base 1: mod_base = OTHER; note = 2'-methoxy
  modified_base 3: mod_base = OTHER; note = 2'-methoxy
  modified_base 5: mod_base = OTHER; note = 2'-methoxy
  modified_base 7..13: mod_base = OTHER; note = 2'-methoxy
  modified_base 15: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 4: mod_base = OTHER; note = 2'-O-tert-butyldimethylsilyl
  modified_base 2: mod_base = OTHER; note = 2'-fluoro
  modified_base 6: mod_base = OTHER; note = 2'-fluoro
  modified_base 14: mod_base = OTHER; note = 2'-fluoro
  modified_base 16: mod_base = OTHER; note = 2'-fluoro
agatggtcttgtctgtctgga

SEQ ID NO: 11 - RNA, 21 nt, synthetic construct
  source 1..21: mol_type = other RNA
  modified_base 1: mod_base = OTHER; note = 2'-methoxy
  modified_base 3: mod_base = OTHER; note = 2'-methoxy
  modified_base 4: mod_base = OTHER; note = 2'-methoxy
  modified_base 7..13: mod_base = OTHER; note = 2'-methoxy
  modified_base 15: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 5: mod_base = OTHER; note = 2'-O-tert-butyldimethylsilyl
  modified_base 2: mod_base = OTHER; note = 2'-fluoro
  modified_base 6: mod_base = OTHER; note = 2'-fluoro
  modified_base 14: mod_base = OTHER; note = 2'-fluoro
  modified_base 16: mod_base = OTHER; note = 2'-fluoro
agatggtcttgtctgtctgga

SEQ ID NO: 12 - RNA, 21 nt, synthetic construct
  source 1..21: mol_type = other RNA
  modified_base 3..5: mod_base = OTHER; note = 2'-methoxy
  modified_base 1: mod_base = OTHER; note = 2'-methoxy
  modified_base 7..13: mod_base = OTHER; note = 2'-methoxy
  modified_base 15: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 6: mod_base = OTHER; note = 2'-O-tert-butyldimethylsilyl
  modified_base 2: mod_base = OTHER; note = 2'-fluoro
  modified_base 14: mod_base = OTHER; note = 2'-fluoro
  modified_base 16: mod_base = OTHER; note = 2'-fluoro
agatggtcttgtctgtctgga

SEQ ID NO: 13 - RNA, 21 nt, synthetic construct
  source 1..21: mol_type = other RNA
  modified_base 3..5: mod_base = OTHER; note = 2'-methoxy
  modified_base 1: mod_base = OTHER; note = 2'-methoxy
  modified_base 8..13: mod_base = OTHER; note = 2'-methoxy
  modified_base 15: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 7: mod_base = OTHER; note = 2'-O-tert-butyldimethylsilyl
  modified_base 2: mod_base = OTHER; note = 2'-fluoro
  modified_base 6: mod_base = OTHER; note = 2'-fluoro
  modified_base 14: mod_base = OTHER; note = 2'-fluoro
  modified_base 16: mod_base = OTHER; note = 2'-fluoro
agatggtcttgtctgtctgga

SEQ ID NO: 14 - RNA, 21 nt, synthetic construct
  source 1..21: mol_type = other RNA
  modified_base 3..5: mod_base = OTHER; note = 2'-methoxy
  modified_base 1: mod_base = OTHER; note = 2'-methoxy
  modified_base 7: mod_base = OTHER; note = 2'-methoxy
  modified_base 9..13: mod_base = OTHER; note = 2'-methoxy
  modified_base 15: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 8: mod_base = OTHER; note = 2'-O-tert-butyldimethylsilyl
  modified_base 2: mod_base = OTHER; note = 2'-fluoro
  modified_base 6: mod_base = OTHER; note = 2'-fluoro
  modified_base 14: mod_base = OTHER; note = 2'-fluoro
  modified_base 16: mod_base = OTHER; note = 2'-fluoro
agatggtcttgtctgtctgga

SEQ ID NO: 15 - RNA, 21 nt, synthetic construct
  source 1..21: mol_type = other RNA
  modified_base 3..5: mod_base = OTHER; note = 2'-methoxy
  modified_base 1: mod_base = OTHER; note = 2'-methoxy
  modified_base 7: mod_base = OTHER; note = 2'-methoxy
  modified_base 8: mod_base = OTHER; note = 2'-methoxy
  modified_base 10..13: mod_base = OTHER; note = 2'-methoxy
  modified_base 15: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 9: mod_base = OTHER; note = 2'-O-tert-butyldimethylsilyl
  modified_base 2: mod_base = OTHER; note = 2'-fluoro
  modified_base 6: mod_base = OTHER; note = 2'-fluoro
  modified_base 14: mod_base = OTHER; note = 2'-fluoro
  modified_base 16: mod_base = OTHER; note = 2'-fluoro
agatggtcttgtctgtctgga

SEQ ID NO: 16 - RNA, 21 nt, synthetic construct
  source 1..21: mol_type = other RNA
  modified_base 1: mod_base = OTHER; note = 2'-methoxy
  modified_base 3..5: mod_base = OTHER; note = 2'-methoxy
  modified_base 15: mod_base = OTHER; note = 2'-methoxy
  modified_base 7..13: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 2: mod_base = OTHER; note = 2'-fluoro
  modified_base 6: mod_base = OTHER; note = 2'-fluoro
  modified_base 14: mod_base = OTHER; note = 2'-fluoro
  modified_base 16: mod_base = OTHER; note = 2'-fluoro
tagttcttggtgctcttggct

SEQ ID NO: 17 - RNA, 21 nt, synthetic construct
  source 1..21: mol_type = other RNA
  modified_base 4: mod_base = OTHER; note = 2'-methoxy
  modified_base 5: mod_base = OTHER; note = 2'-methoxy
  modified_base 7..13: mod_base = OTHER; note = 2'-methoxy
  modified_base 15: mod_base = OTHER; note = 2'-methoxy
  modified_base 1: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 3: mod_base = OTHER; note = 2'-O-tert-butyldimethylsilyl
  modified_base 2: mod_base = OTHER; note = 2'-fluoro
  modified_base 6: mod_base = OTHER; note = 2'-fluoro
  modified_base 14: mod_base = OTHER; note = 2'-fluoro
  modified_base 16: mod_base = OTHER; note = 2'-fluoro
tagttcttggtgctcttggct

SEQ ID NO: 18 - RNA, 21 nt, synthetic construct
  source 1..21: mol_type = other RNA
  modified_base 3: mod_base = OTHER; note = 2'-methoxy
  modified_base 5: mod_base = OTHER; note = 2'-methoxy
  modified_base 7..13: mod_base = OTHER; note = 2'-methoxy
  modified_base 15: mod_base = OTHER; note = 2'-methoxy
  modified_base 1: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 4: mod_base = OTHER; note = 2'-O-tert-butyldimethylsilyl
  modified_base 2: mod_base = OTHER; note = 2'-fluoro
  modified_base 6: mod_base = OTHER; note = 2'-fluoro
  modified_base 14: mod_base = OTHER; note = 2'-fluoro
  modified_base 16: mod_base = OTHER; note = 2'-fluoro
tagttcttggtgctcttggct

SEQ ID NO: 19 - RNA, 21 nt, synthetic construct
  source 1..21: mol_type = other RNA
  modified_base 3: mod_base = OTHER; note = 2'-methoxy
  modified_base 4: mod_base = OTHER; note = 2'-methoxy
  modified_base 7..13: mod_base = OTHER; note = 2'-methoxy
  modified_base 15: mod_base = OTHER; note = 2'-methoxy
  modified_base 1: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 5: mod_base = OTHER; note = 2'-O-tert-butyldimethylsilyl
  modified_base 2: mod_base = OTHER; note = 2'-fluoro
  modified_base 6: mod_base = OTHER; note = 2'-fluoro
  modified_base 14: mod_base = OTHER; note = 2'-fluoro
  modified_base 16: mod_base = OTHER; note = 2'-fluoro
tagttcttggtgctcttggct

SEQ ID NO: 20 - RNA, 21 nt, synthetic construct
  source 1..21: mol_type = other RNA
  modified_base 3..5: mod_base = OTHER; note = 2'-methoxy
  modified_base 7..13: mod_base = OTHER; note = 2'-methoxy
  modified_base 15: mod_base = OTHER; note = 2'-methoxy
  modified_base 1: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 6: mod_base = OTHER; note = 2'-O-tert-butyldimethylsilyl
  modified_base 2: mod_base = OTHER; note = 2'-fluoro
  modified_base 14: mod_base = OTHER; note = 2'-fluoro
  modified_base 16: mod_base = OTHER; note = 2'-fluoro
tagttcttggtgctcttggct

SEQ ID NO: 21 - RNA, 21 nt, synthetic construct
  source 1..21: mol_type = other RNA
  modified_base 1: mod_base = OTHER; note = 2'-methoxy
  modified_base 3..5: mod_base = OTHER; note = 2'-methoxy
  modified_base 8..13: mod_base = OTHER; note = 2'-methoxy
  modified_base 15: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 7: mod_base = OTHER; note = 2'-O-tert-butyldimethylsilyl
  modified_base 2: mod_base = OTHER; note = 2'-fluoro
  modified_base 6: mod_base = OTHER; note = 2'-fluoro
  modified_base 14: mod_base = OTHER; note = 2'-fluoro
  modified_base 16: mod_base = OTHER; note = 2'-fluoro
tagttcttggtgctcttggct

SEQ ID NO: 22 - RNA, 21 nt, synthetic construct
  source 1..21: mol_type = other RNA
  modified_base 1: mod_base = OTHER; note = 2'-methoxy
  modified_base 3..5: mod_base = OTHER; note = 2'-methoxy
  modified_base 7: mod_base = OTHER; note = 2'-methoxy
  modified_base 9..13: mod_base = OTHER; note = 2'-methoxy
  modified_base 15: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 8: mod_base = OTHER; note = 2'-O-tert-butyldimethylsilyl
  modified_base 2: mod_base = OTHER; note = 2'-fluoro
  modified_base 6: mod_base = OTHER; note = 2'-fluoro
  modified_base 14: mod_base = OTHER; note = 2'-fluoro
  modified_base 16: mod_base = OTHER; note = 2'-fluoro
tagttcttggtgctcttggct

SEQ ID NO: 23 - RNA, 21 nt, synthetic construct
  source 1..21: mol_type = other RNA
  modified_base 1: mod_base = OTHER; note = 2'-methoxy
  modified_base 3..5: mod_base = OTHER; note = 2'-methoxy
  modified_base 7: mod_base = OTHER; note = 2'-methoxy
  modified_base 8: mod_base = OTHER; note = 2'-methoxy
  modified_base 10..13: mod_base = OTHER; note = 2'-methoxy
  modified_base 15: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 9: mod_base = OTHER; note = 2'-O-tert-butyldimethylsilyl
  modified_base 2: mod_base = OTHER; note = 2'-fluoro
  modified_base 6: mod_base = OTHER; note = 2'-fluoro
  modified_base 14: mod_base = OTHER; note = 2'-fluoro
  modified_base 16: mod_base = OTHER; note = 2'-fluoro
tagttcttggtgctcttggct

SEQ ID NO: 24 - RNA, 21 nt, synthetic construct
  source 1..21: mol_type = other RNA
  modified_base 1: mod_base = OTHER; note = 2'-methoxy
  modified_base 3..5: mod_base = OTHER; note = 2'-methoxy
  modified_base 7..13: mod_base = OTHER; note = 2'-methoxy
  modified_base 15: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 2: mod_base = OTHER; note = 2'-fluoro
  modified_base 6: mod_base = OTHER; note = 2'-fluoro
  modified_base 14: mod_base = OTHER; note = 2'-fluoro
  modified_base 16: mod_base = OTHER; note = 2'-fluoro
cagaatccagtccacgtactt

SEQ ID NO: 25 - RNA, 21 nt, synthetic construct
  source 1..21: mol_type = other RNA
  modified_base 1: mod_base = OTHER; note = 2'-methoxy
  modified_base 3..4: mod_base = OTHER; note = 2'-methoxy
  modified_base 7..13: mod_base = OTHER; note = 2'-methoxy
  modified_base 15: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 5: mod_base = OTHER; note = 2'-O-triisopropylmethoxysilane
  modified_base 2: mod_base = OTHER; note = 2'-fluoro
  modified_base 6: mod_base = OTHER; note = 2'-fluoro
  modified_base 14: mod_base = OTHER; note = 2'-fluoro
  modified_base 16: mod_base = OTHER; note = 2'-fluoro
cagaatccagtccacgtactt

SEQ ID NO: 26 - RNA, 21 nt, synthetic construct
  source 1..21: mol_type = other RNA
  modified_base 1: mod_base = OTHER; note = 2'-methoxy
  modified_base 3..5: mod_base = OTHER; note = 2'-methoxy
  modified_base 7..13: mod_base = OTHER; note = 2'-methoxy
  modified_base 15: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 6: mod_base = OTHER; note = 2'-O-triisopropylmethoxysilane
  modified_base 2: mod_base = OTHER; note = 2'-fluoro
  modified_base 14: mod_base = OTHER; note = 2'-fluoro
  modified_base 16: mod_base = OTHER; note = 2'-fluoro
cagaatccagtccacgtactt

SEQ ID NO: 27 - RNA, 21 nt, synthetic construct
  source 1..21: mol_type = other RNA
  modified_base 1: mod_base = OTHER; note = 2'-methoxy
  modified_base 3..5: mod_base = OTHER; note = 2'-methoxy
  modified_base 8..13: mod_base = OTHER; note = 2'-methoxy
  modified_base 15: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 7: mod_base = OTHER; note = 2'-O-triisopropylmethoxysilane
  modified_base 2: mod_base = OTHER; note = 2'-fluoro
  modified_base 6: mod_base = OTHER; note = 2'-fluoro
  modified_base 14: mod_base = OTHER; note = 2'-fluoro
  modified_base 16: mod_base = OTHER; note = 2'-fluoro
cagaatccagtccacgtactt

SEQ ID NO: 28 - RNA, 21 nt, synthetic construct
  source 1..21: mol_type = other RNA
  modified_base 1: mod_base = OTHER; note = 2'-methoxy
  modified_base 3..5: mod_base = OTHER; note = 2'-methoxy
  modified_base 7: mod_base = OTHER; note = 2'-methoxy
  modified_base 9..13: mod_base = OTHER; note = 2'-methoxy
  modified_base 15: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 8: mod_base = OTHER; note = 2'-O-triisopropylmethoxysilane
  modified_base 2: mod_base = OTHER; note = 2'-fluoro
  modified_base 6: mod_base = OTHER; note = 2'-fluoro
  modified_base 14: mod_base = OTHER; note = 2'-fluoro
  modified_base 16: mod_base = OTHER; note = 2'-fluoro
cagaatccagtccacgtactt

SEQ ID NO: 29 - RNA, 21 nt, synthetic construct
  source 1..21: mol_type = other RNA
  modified_base 1: mod_base = OTHER; note = 2'-methoxy
  modified_base 3..5: mod_base = OTHER; note = 2'-methoxy
  modified_base 7: mod_base = OTHER; note = 2'-methoxy
  modified_base 8: mod_base = OTHER; note = 2'-methoxy
  modified_base 10..13: mod_base = OTHER; note = 2'-methoxy
  modified_base 15: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 9: mod_base = OTHER; note = 2'-O-triisopropylmethoxysilane
  modified_base 2: mod_base = OTHER; note = 2'-fluoro
  modified_base 6: mod_base = OTHER; note = 2'-fluoro
  modified_base 14: mod_base = OTHER; note = 2'-fluoro
  modified_base 16: mod_base = OTHER; note = 2'-fluoro
cagaatccagtccacgtactt

SEQ ID NO: 30 - RNA, 21 nt, synthetic construct
  source 1..21: mol_type = other RNA
  modified_base 1: mod_base = OTHER; note = 2'-methoxy
  modified_base 3..5: mod_base = OTHER; note = 2'-methoxy
  modified_base 7..13: mod_base = OTHER; note = 2'-methoxy
  modified_base 15: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 2: mod_base = OTHER; note = 2'-fluoro
  modified_base 6: mod_base = OTHER; note = 2'-fluoro
  modified_base 14: mod_base = OTHER; note = 2'-fluoro
  modified_base 16: mod_base = OTHER; note = 2'-fluoro
agatggtcttgtctgtctgga

SEQ ID NO: 31 - RNA, 21 nt, synthetic construct
  source 1..21: mol_type = other RNA
  modified_base 1: mod_base = OTHER; note = 2'-methoxy
  modified_base 3..5: mod_base = OTHER; note = 2'-methoxy
  modified_base 8..13: mod_base = OTHER; note = 2'-methoxy
  modified_base 15: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 7: mod_base = OTHER; note = 2'-O-triisopropylmethoxysilane
  modified_base 2: mod_base = OTHER; note = 2'-fluoro
  modified_base 6: mod_base = OTHER; note = 2'-fluoro
  modified_base 14: mod_base = OTHER; note = 2'-fluoro
  modified_base 16: mod_base = OTHER; note = 2'-fluoro
agatggtcttgtctgtctgga

SEQ ID NO: 32 - RNA, 21 nt, synthetic construct
  source 1..21: mol_type = other RNA
  modified_base 1: mod_base = OTHER; note = 2'-methoxy
  modified_base 3..5: mod_base = OTHER; note = 2'-methoxy
  modified_base 7: mod_base = OTHER; note = 2'-methoxy
  modified_base 8: mod_base = OTHER; note = 2'-methoxy
  modified_base 10..13: mod_base = OTHER; note = 2'-methoxy
  modified_base 15: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 9: mod_base = OTHER; note = 2'-O-triisopropylmethoxysilane
  modified_base 2: mod_base = OTHER; note = 2'-fluoro
  modified_base 6: mod_base = OTHER; note = 2'-fluoro
  modified_base 14: mod_base = OTHER; note = 2'-fluoro
  modified_base 16: mod_base = OTHER; note = 2'-fluoro
agatggtcttgtctgtctgga

SEQ ID NO: 33 - RNA, 21 nt, synthetic construct
  source 1..21: mol_type = other RNA
  modified_base 1: mod_base = OTHER; note = 2'-methoxy
  modified_base 3..5: mod_base = OTHER; note = 2'-methoxy
  modified_base 7..13: mod_base = OTHER; note = 2'-methoxy
  modified_base 15: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 2: mod_base = OTHER; note = 2'-fluoro
  modified_base 6: mod_base = OTHER; note = 2'-fluoro
  modified_base 14: mod_base = OTHER; note = 2'-fluoro
  modified_base 16: mod_base = OTHER; note = 2'-fluoro
tagttcttggtgctcttggct

SEQ ID NO: 34 - RNA, 21 nt, Synthetic construct
  source 1..21: mol_type = other RNA
  modified_base 1: mod_base = OTHER; note = 2'-methoxy
  modified_base 4: mod_base = OTHER; note = 2'-methoxy
  modified_base 5: mod_base = OTHER; note = 2'-methoxy
  modified_base 7..13: mod_base = OTHER; note = 2'-methoxy
  modified_base 15: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 3: mod_base = OTHER; note = 2'-O-tert-butyldimethylsilyl
  modified_base 2: mod_base = OTHER; note = 2'-fluoro
  modified_base 6: mod_base = OTHER; note = 2'-fluoro
  modified_base 14: mod_base = OTHER; note = 2'-fluoro
  modified_base 16: mod_base = OTHER; note = 2'-fluoro
tagttcttggtgctcttggct

SEQ ID NO: 35 - RNA, 21 nt, synthetic construct
  source 1..21: mol_type = other RNA
  modified_base 1: mod_base = OTHER; note = 2'-methoxy
  modified_base 3: mod_base = OTHER; note = 2'-methoxy
  modified_base 5: mod_base = OTHER; note = 2'-methoxy
  modified_base 7..13: mod_base = OTHER; note = 2'-methoxy
  modified_base 15: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 4: mod_base = OTHER; note = 2'-O-tert-butyldimethylsilyl
  modified_base 2: mod_base = OTHER; note = 2'-fluoro
  modified_base 6: mod_base = OTHER; note = 2'-fluoro
  modified_base 14: mod_base = OTHER; note = 2'-fluoro
  modified_base 16: mod_base = OTHER; note = 2'-fluoro
tagttcttggtgctcttggct

SEQ ID NO: 36 - RNA, 21 nt, synthetic construct
  source 1..21: mol_type = other RNA
  modified_base 1: mod_base = OTHER; note = 2'-methoxy
  modified_base 3: mod_base = OTHER; note = 2'-methoxy
  modified_base 4: mod_base = OTHER; note = 2'-methoxy
  modified_base 7..13: mod_base = OTHER; note = 2'-methoxy
  modified_base 15: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 5: mod_base = OTHER; note = 2'-O-tert-butyldimethylsilyl
  modified_base 2: mod_base = OTHER; note = 2'-fluoro
  modified_base 6: mod_base = OTHER; note = 2'-fluoro
  modified_base 14: mod_base = OTHER; note = 2'-fluoro
  modified_base 16: mod_base = OTHER; note = 2'-fluoro
tagttcttggtgctcttggct

SEQ ID NO: 37 - RNA, 21 nt, synthetic construct
  source 1..21: mol_type = other RNA
  modified_base 1: mod_base = OTHER; note = 2'-methoxy
  modified_base 3..5: mod_base = OTHER; note = 2'-methoxy
  modified_base 7..13: mod_base = OTHER; note = 2'-methoxy
  modified_base 15: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 6: mod_base = OTHER; note = 2'-O-tert-butyldimethylsilyl
  modified_base 2: mod_base = OTHER; note = 2'-fluoro
  modified_base 14: mod_base = OTHER; note = 2'-fluoro
  modified_base 16: mod_base = OTHER; note = 2'-fluoro
tagttcttggtgctcttggct

SEQ ID NO: 38 - RNA, 21 nt, synthetic construct
  source 1..21: mol_type = other RNA
  modified_base 1: mod_base = OTHER; note = 2'-methoxy
  modified_base 3..5: mod_base = OTHER; note = 2'-methoxy
  modified_base 8..13: mod_base = OTHER; note = 2'-methoxy
  modified_base 15: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 7: mod_base = OTHER; note = 2'-O-tert-butyldimethylsilyl
  modified_base 2: mod_base = OTHER; note = 2'-fluoro
  modified_base 6: mod_base = OTHER; note = 2'-fluoro
  modified_base 14: mod_base = OTHER; note = 2'-fluoro
  modified_base 16: mod_base = OTHER; note = 2'-fluoro
tagttcttggtgctcttggct

SEQ ID NO: 39 - RNA, 21 nt, synthetic construct
  source 1..21: mol_type = other RNA
  modified_base 1: mod_base = OTHER; note = 2'-methoxy
  modified_base 3..5: mod_base = OTHER; note = 2'-methoxy
  modified_base 7: mod_base = OTHER; note = 2'-methoxy
  modified_base 9..13: mod_base = OTHER; note = 2'-methoxy
  modified_base 15: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 8: mod_base = OTHER; note = 2'-O-tert-butyldimethylsilyl
  modified_base 2: mod_base = OTHER; note = 2'-fluoro
  modified_base 6: mod_base = OTHER; note = 2'-fluoro
  modified_base 14: mod_base = OTHER; note = 2'-fluoro
  modified_base 16: mod_base = OTHER; note = 2'-fluoro
tagttcttggtgctcttggct

SEQ ID NO: 40 - RNA, 21 nt, synthetic construct
  source 1..21: mol_type = other RNA
  modified_base 1: mod_base = OTHER; note = 2'-methoxy
  modified_base 3..5: mod_base = OTHER; note = 2'-methoxy
  modified_base 7: mod_base = OTHER; note = 2'-methoxy
  modified_base 8: mod_base = OTHER; note = 2'-methoxy
  modified_base 10..13: mod_base = OTHER; note = 2'-methoxy
  modified_base 15: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 9: mod_base = OTHER; note = 2'-O-tert-butyldimethylsilyl
  modified_base 2: mod_base = OTHER; note = 2'-fluoro
  modified_base 6: mod_base = OTHER; note = 2'-fluoro
  modified_base 14: mod_base = OTHER; note = 2'-fluoro
  modified_base 16: mod_base = OTHER; note = 2'-fluoro
tagttcttggtgctcttggct

SEQ ID NO: 41 - RNA, 19 nt, synthetic construct
  source 1..19: mol_type = other RNA
ccaagagcaccaagaacta

SEQ ID NO: 42 - RNA, 21 nt, synthetic construct
  source 1..21: mol_type = other RNA
tagttcttggtgctcttggct

SEQ ID NO: 43 - RNA, 19 nt, synthetic construct
  source 1..19: mol_type = other RNA
cagacagacaagaccatct

SEQ ID NO: 44 - RNA, 21 nt, synthetic construct
  source 1..21: mol_type = other RNA
agatggtcttgtctgtctgga

SEQ ID NO: 45 - RNA, 19 nt, synthetic construct
  source 1..19: mol_type = other RNA
gtacgtggactggattctg

SEQ ID NO: 46 - RNA, 21 nt, synthetic construct
  source 1..21: mol_type = other RNA
cagaatccagtccacgtactt

SEQ ID NO: 47 - DNA, 20 bp, synthetic construct
  source 1..20: mol_type = other DNA
aagcgcattccgattgagga

SEQ ID NO: 48 - DNA, 20 bp, synthetic construct
  source 1..20: mol_type = other DNA
cctgagtgcaagatgacggt

SEQ ID NO: 49 - DNA, 20 bp, synthetic construct
  source 1..20: mol_type = other DNA
cctgagtgcaagatgacggt

SEQ ID NO: 50 - DNA, 19 bp, synthetic construct
  source 1..19: mol_type = other DNA
ccagcatcgccccacttga

SEQ ID NO: 51 - DNA, 23 bp, synthetic construct
  source 1..23: mol_type = other DNA
gaggagcagctaaccaacttaat

SEQ ID NO: 52 - DNA, 23 bp, synthetic construct
  source 1..23: mol_type = other DNA
tctgcatgtgctgttgacttaat

SEQ ID NO: 53 - DNA, 19 bp, synthetic construct
  source 1..19: mol_type = other DNA
tgcaccaccaactgcttag

SEQ ID NO: 54 - DNA, 19 bp, synthetic construct
  source 1..19: mol_type = other DNA
ggatgcagggatgatgttc

SEQ ID NO: 55 - DNA, 23 bp, synthetic construct
  source 1..23: mol_type = other DNA
gccctgttaaaactggaatcagc

SEQ ID NO: 56 - DNA, 23 bp, synthetic construct
  source 1..23: mol_type = other DNA
gccctgttaaaactggaatcagc

SEQ ID NO: 57 - RNA, 19 nt, synthetic construct
  source 1..19: mol_type = other RNA
  modified_base 1..6: mod_base = OTHER; note = 2'-methoxy
  modified_base 10..19: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 7: mod_base = OTHER; note = 2'-fluoro
  modified_base 9: mod_base = OTHER; note = 2'-fluoro
  modified_base 19: mod_base = OTHER; note = GalNA
  modified_base 8: mod_base = OTHER; note = deoxyribonucleotide
cagacagacaagaccatct

SEQ ID NO: 58 - RNA, 19 nt, synthetic construct
  source 1..19: mol_type = other RNA
  modified_base 1..6: mod_base = OTHER; note = 2'-methoxy
  modified_base 10..19: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 7: mod_base = OTHER; note = 2'-fluoro
  modified_base 9: mod_base = OTHER; note = 2'-fluoro
  modified_base 8: mod_base = OTHER; note = deoxyribonucleotide
ccaagagcaccaagaacta

SEQ ID NO: 59 - RNA, 19 nt, synthetic construct
  source 1..19: mol_type = other RNA
  modified_base 1..6: mod_base = OTHER; note = 2'-methoxy
  modified_base 10..19: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 7: mod_base = OTHER; note = 2'-fluoro
  modified_base 9: mod_base = OTHER; note = 2'-fluoro
  modified_base 19: mod_base = OTHER; note = GalNA
  modified_base 8: mod_base = OTHER; note = deoxyribonucleotide
ttctccgaacgtgtcacgt

SEQ ID NO: 60 - RNA, 19 nt, synthetic construct
  source 1..19: mol_type = other RNA
  modified_base 1..6: mod_base = OTHER; note = 2'-methoxy
  modified_base 11..19: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 7..10: mod_base = OTHER; note = 2'-fluoro
ccaagagcaccaagaacta

SEQ ID NO: 61 - RNA, 21 nt, synthetic construct
  source 1..21: mol_type = other RNA
  modified_base 1: mod_base = OTHER; note = 2'-methoxy
  modified_base 3..5: mod_base = OTHER; note = 2'-methoxy
  modified_base 7: mod_base = OTHER; note = 2'-methoxy
  modified_base 8: mod_base = OTHER; note = 2'-methoxy
  modified_base 10..13: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 15: mod_base = OTHER; note = O-methoxyethyl
  modified_base 2: mod_base = OTHER; note = 2'-fluoro
  modified_base 6: mod_base = OTHER; note = 2'-fluoro
  modified_base 9: mod_base = OTHER; note = 2'-fluoro
  modified_base 14: mod_base = OTHER; note = 2'-fluoro
  modified_base 16: mod_base = OTHER; note = 2'-fluoro
tagttcttggtgctcttggct

SEQ ID NO: 62 - RNA, 21 nt, synthetic construct
  source 1..21: mol_type = other RNA
  modified_base 1: mod_base = OTHER; note = 2'-methoxy
  modified_base 4: mod_base = OTHER; note = 2'-methoxy
  modified_base 5: mod_base = OTHER; note = 2'-methoxy
  modified_base 7: mod_base = OTHER; note = 2'-methoxy
  modified_base 8: mod_base = OTHER; note = 2'-methoxy
  modified_base 10..13: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 15: mod_base = OTHER; note = O-methoxyethyl
  modified_base 2: mod_base = OTHER; note = 2'-fluoro
  modified_base 6: mod_base = OTHER; note = 2'-fluoro
  modified_base 9: mod_base = OTHER; note = 2'-fluoro
  modified_base 14: mod_base = OTHER; note = 2'-fluoro
  modified_base 16: mod_base = OTHER; note = 2'-fluoro
  modified_base 3: mod_base = OTHER; note = 2'-O-tert-butyldimethylsilyl
tagttcttggtgctcttggct

SEQ ID NO: 63 - RNA, 21 nt, synthetic construct
  source 1..21: mol_type = other RNA
  modified_base 1: mod_base = OTHER; note = 2'-methoxy
  modified_base 3: mod_base = OTHER; note = 2'-methoxy
  modified_base 5: mod_base = OTHER; note = 2'-methoxy
  modified_base 7: mod_base = OTHER; note = 2'-methoxy
  modified_base 8: mod_base = OTHER; note = 2'-methoxy
  modified_base 10..13: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 15: mod_base = OTHER; note = O-methoxyethyl
  modified_base 2: mod_base = OTHER; note = 2'-fluoro
  modified_base 6: mod_base = OTHER; note = 2'-fluoro
  modified_base 9: mod_base = OTHER; note = 2'-fluoro
  modified_base 14: mod_base = OTHER; note = 2'-fluoro
  modified_base 16: mod_base = OTHER; note = 2'-fluoro
  modified_base 4: mod_base = OTHER; note = 2'-O-tert-butyldimethylsilyl
tagttcttggtgctcttggct

SEQ ID NO: 64 - RNA, 21 nt, synthetic construct
  source 1..21: mol_type = other RNA
  modified_base 1: mod_base = OTHER; note = 2'-methoxy
  modified_base 3: mod_base = OTHER; note = 2'-methoxy
  modified_base 4: mod_base = OTHER; note = 2'-methoxy
  modified_base 7: mod_base = OTHER; note = 2'-methoxy
  modified_base 8: mod_base = OTHER; note = 2'-methoxy
  modified_base 10..13: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 15: mod_base = OTHER; note = O-methoxyethyl
  modified_base 2: mod_base = OTHER; note = 2'-fluoro
  modified_base 6: mod_base = OTHER; note = 2'-fluoro
  modified_base 9: mod_base = OTHER; note = 2'-fluoro
  modified_base 14: mod_base = OTHER; note = 2'-fluoro
  modified_base 16: mod_base = OTHER; note = 2'-fluoro
  modified_base 5: mod_base = OTHER; note = 2'-O-tert-butyldimethylsilyl
tagttcttggtgctcttggct

SEQ ID NO: 65 - RNA, 21 nt, synthetic construct
  source 1..21: mol_type = other RNA
  modified_base 1: mod_base = OTHER; note = 2'-methoxy
  modified_base 3..5: mod_base = OTHER; note = 2'-methoxy
  modified_base 7: mod_base = OTHER; note = 2'-methoxy
  modified_base 8: mod_base = OTHER; note = 2'-methoxy
  modified_base 10..13: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19..20: mod_base = OTHER; note = phosphorothioate bond
  modified_base 20^21: mod_base = OTHER; note = phosphorothioate bond
  modified_base 15: mod_base = OTHER; note = O-methoxyethyl
  modified_base 2: mod_base = OTHER; note = 2'-fluoro
  modified_base 9: mod_base = OTHER; note = 2'-fluoro
  modified_base 14: mod_base = OTHER; note = 2'-fluoro
  modified_base 16: mod_base = OTHER; note = 2'-fluoro
  modified_base 6: mod_base = OTHER; note = 2'-O-tert-butyldimethylsilyl
tagttcttggtgctcttggct

SEQ ID NO: 66 - RNA, 21 nt, synthetic construct
  source 1..21: mol_type = other RNA
  modified_base 1: mod_base = OTHER; note = 2'-methoxy
  modified_base 3..5: mod_base = OTHER; note = 2'-methoxy
  modified_base 8: mod_base = OTHER; note = 2'-methoxy
  modified_base 10..13: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 15: mod_base = OTHER; note = O-methoxyethyl
  modified_base 2: mod_base = OTHER; note = 2'-fluoro
  modified_base 6: mod_base = OTHER; note = 2'-fluoro
  modified_base 9: mod_base = OTHER; note = 2'-fluoro
  modified_base 14: mod_base = OTHER; note = 2'-fluoro
  modified_base 16: mod_base = OTHER; note = 2'-fluoro
  modified_base 7: mod_base = OTHER; note = 2'-O-tert-butyldimethylsilyl
tagttcttggtgctcttggct

SEQ ID NO: 67 - RNA, 21 nt, synthetic construct
  source 1..21: mol_type = other RNA
  modified_base 1: mod_base = OTHER; note = 2'-methoxy
  modified_base 3..5: mod_base = OTHER; note = 2'-methoxy
  modified_base 7: mod_base = OTHER; note = 2'-methoxy
  modified_base 10..13: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 15: mod_base = OTHER; note = O-methoxyethyl
  modified_base 2: mod_base = OTHER; note = 2'-fluoro
  modified_base 6: mod_base = OTHER; note = 2'-fluoro
  modified_base 9: mod_base = OTHER; note = 2'-fluoro
  modified_base 14: mod_base = OTHER; note = 2'-fluoro
  modified_base 16: mod_base = OTHER; note = 2'-fluoro
  modified_base 8: mod_base = OTHER; note = 2'-O-tert-butyldimethylsilyl
tagttcttggtgctcttggct

SEQ ID NO: 68 - RNA, 21 nt, synthetic construct
  source 1..21: mol_type = other RNA
  modified_base 1: mod_base = OTHER; note = 2'-methoxy
  modified_base 3..5: mod_base = OTHER; note = 2'-methoxy
  modified_base 7: mod_base = OTHER; note = 2'-methoxy
  modified_base 8: mod_base = OTHER; note = 2'-methoxy
  modified_base 10..13: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 15: mod_base = OTHER; note = O-methoxyethyl
  modified_base 2: mod_base = OTHER; note = 2'-fluoro
  modified_base 6: mod_base = OTHER; note = 2'-fluoro
  modified_base 14: mod_base = OTHER; note = 2'-fluoro
  modified_base 16: mod_base = OTHER; note = 2'-fluoro
  modified_base 9: mod_base = OTHER; note = 2'-O-tert-butyldimethylsilyl
tagttcttggtgctcttggct

SEQ ID NO: 69 - RNA, 21 nt, synthetic construct
  source 1..21: mol_type = other RNA
  modified_base 1: mod_base = OTHER; note = 2'-methoxy
  modified_base 3: mod_base = OTHER; note = 2'-methoxy
  modified_base 5: mod_base = OTHER; note = 2'-methoxy
  modified_base 7: mod_base = OTHER; note = 2'-methoxy
  modified_base 8: mod_base = OTHER; note = 2'-methoxy
  modified_base 10..13: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 15: mod_base = OTHER; note = O-methoxyethyl
  modified_base 2: mod_base = OTHER; note = 2'-fluoro
  modified_base 6: mod_base = OTHER; note = 2'-fluoro
  modified_base 9: mod_base = OTHER; note = 2'-fluoro
  modified_base 14: mod_base = OTHER; note = 2'-fluoro
  modified_base 16: mod_base = OTHER; note = 2'-fluoro
  modified_base 4: mod_base = OTHER; note = 2'-O-triisopropylmethoxysilane
tagttcttggtgctcttggct

SEQ ID NO: 70 - RNA, 21 nt, synthetic construct
  source 1..21: mol_type = other RNA
  modified_base 1: mod_base = OTHER; note = 2'-methoxy
  modified_base 3: mod_base = OTHER; note = 2'-methoxy
  modified_base 4: mod_base = OTHER; note = 2'-methoxy
  modified_base 7: mod_base = OTHER; note = 2'-methoxy
  modified_base 8: mod_base = OTHER; note = 2'-methoxy
  modified_base 10..13: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 15: mod_base = OTHER; note = O-methoxyethyl
  modified_base 2: mod_base = OTHER; note = 2'-fluoro
  modified_base 6: mod_base = OTHER; note = 2'-fluoro
  modified_base 9: mod_base = OTHER; note = 2'-fluoro
  modified_base 14: mod_base = OTHER; note = 2'-fluoro
  modified_base 16: mod_base = OTHER; note = 2'-fluoro
  modified_base 5: mod_base = OTHER; note = 2'-O-triisopropylmethoxysilane
tagttcttggtgctcttggct

SEQ ID NO: 71 - RNA, 21 nt, synthetic construct
  source 1..21: mol_type = other RNA
  modified_base 1: mod_base = OTHER; note = 2'-methoxy
  modified_base 3..5: mod_base = OTHER; note = 2'-methoxy
  modified_base 8: mod_base = OTHER; note = 2'-methoxy
  modified_base 10..13: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 15: mod_base = OTHER; note = O-methoxyethyl
  modified_base 2: mod_base = OTHER; note = 2'-fluoro
  modified_base 6: mod_base = OTHER; note = 2'-fluoro
  modified_base 9: mod_base = OTHER; note = 2'-fluoro
  modified_base 14: mod_base = OTHER; note = 2'-fluoro
  modified_base 16: mod_base = OTHER; note = 2'-fluoro
  modified_base 7: mod_base = OTHER; note = 2'-O-triisopropylmethoxysilane
tagttcttggtgctcttggct

SEQ ID NO: 72 - RNA, 21 nt, synthetic construct
  source 1..21: mol_type = other RNA
  modified_base 1: mod_base = OTHER; note = 2'-methoxy
  modified_base 3..5: mod_base = OTHER; note = 2'-methoxy
  modified_base 7: mod_base = OTHER; note = 2'-methoxy
  modified_base 10..13: mod_base = OTHER; note = 2'-methoxy
  modified_base 17..21: mod_base = OTHER; note = 2'-methoxy
  modified_base 1^2: mod_base = OTHER; note = phosphorothioate bond
  modified_base 2^3: mod_base = OTHER; note = phosphorothioate bond
  modified_base 19^20: mod_base = OTHER; note = phosphorothioate bond
  modified_base 20^21: mod_base = OTHER; note = phosphorothioate bond
  modified_base 15: mod_base = OTHER; note = O-methoxyethyl
  modified_base 2: mod_base = OTHER; note = 2'-fluoro
  modified_base 6: mod_base = OTHER; note = 2'-fluoro
  modified_base 9: mod_base = OTHER; note = 2'-fluoro
  modified_base 14: mod_base = OTHER; note = 2'-fluoro
  modified_base 16: mod_base = OTHER; note = 2'-fluoro
  modified_base 8: mod_base = OTHER; note = 2'-O-triisopropylmethoxysilane
tagttcttggtgctcttggct

SEQ ID NO: 73 - DNA, 21 bp, synthetic construct
  source 1..21: mol_type = other DNA
agccaagagcaccaagaacta

SEQ ID NO: 74 - DNA, 21 bp, synthetic construct
  source 1..21: mol_type = other DNA
ctaacctctacacaagaacta